(19) United States
(12) Patent Application Publication
(10) Pub. No.: US 2024/0042045 A1
(43) Pub. Date: Feb. 8, 2024

(54) NANOPARTICLES CONTAINING MULTIPLE CLEAVABLE PRODUGS FOR CANCER THERAPY

(71) Applicant: The University of Chicago, Chicago, IL (US)

(72) Inventors: Wenbin Lin, Chicago (US); Xiaomin Jiang, Chicago (US); Wenbo Han, Chicago (US); Xuanyu Feng, Chicago (US)

(21) Appl. No.: 18/022,296

(22) PCT Filed: Aug. 23, 2021
(86) PCT No.: PCT/US2021/047176

§ 371 Date: Feb. 21, 2023

Related U.S. Application Data
(60) Provisional application No. 63/068,800, filed on Aug. 21, 2020.

(51) Int. Cl. A61K 47/54; A61K 47/69; A61K 45/06; A61P 35/00; C07J 43/00

(52) U.S. Cl. CPC A61K 47/554; A61K 47/6929; A61K 45/06; A61P 35/00; C07J 43/003; B82Y 5/00

(57) ABSTRACT

Prodrugs that target the low-density lipoprotein receptor (LDLR) and that comprise acid and/or enzyme cleavable acetal- or oxybenzyloxy-linked carbonate or carbamate bonds are described. Also described are core-shell nanoparticles comprising metal-organic framework cores or nanoscale metal bisphosphate coordination polymer cores, and lipid coating layers containing the prodrugs. The nanoparticle core can optionally contain one or more hydrophilic chemotherapeutic agents. The prodrugs and nanoparticles can be used in methods of treating cancer. For instance, the presently disclosed nanoparticles can be used for the co-delivery of multiple chemotherapeutic agents in methods providing increased accumulation of chemotherapeutic agents to a tumor compared to delivery of mixtures of free chemotherapeutic agents.

### RELATED APPLICATIONS

The presently disclosed subject matter claims the benefit of U.S. Provisional Patent Application Ser. No. 63/068,800, filed Aug. 21, 2020, the disclosure of which is incorporated herein by reference in its entirety.

### STATEMENT OF GOVERNMENT INTEREST

This invention was made with government support under Grant Numbers CA223184 and CA216436 awarded by The National Institutes of Health. The government has certain rights to this invention.

### TECHNICAL FIELD

The presently disclosed subject matter provides prodrugs (e.g., prodrugs of chemotherapeutic agents) comprising monovalent drug moieties bound to monovalent lipid moieties via cleavable carbonate or carbamate linkers. The prodrugs can target the low-density lipoprotein receptor (LDLR). The presently disclosed subject matter also provides nanoparticles comprising the prodrugs. The nanoparticles can be core-shell nanoparticles that comprise, for example, (i) a lipid coating layer containing a prodrug comprising a monovalent lipid moiety and a cleavable carbonate or carbamate linker and (ii) a nanoscale coordination polymer (NCP) nanoparticle core, which can itself optionally comprise one or more chemotherapeutic agents or analogues or prodrugs thereof. The prodrugs and nanoparticles can be used in treating cancer. In some embodiments, the nanoparticle-based compositions of the presently disclosed subject matter can provide enhanced anti-cancer effects by combining multiple treatment modalities in a variety of cancers.

### Abbreviations

° C.=degrees Celsius
%=percentage
μg=microgram
μl or μL=microliter
M=micromolar
5-FU=5-fluorouracil
ApoB-100=apolipoprotein B100
AS ODN=antisense oligonucleotide
AUC=area under the curve
Bcl-2=B-cell lymphoma 2
Ca=calcium
Chol=cholesterol
CisPt=cisplatin
CPT=camptothecin
dach=trans-1,2-diaminocyclohexane)
DCM=dichloromethane
DHA=dihydroartemisinin
DLS=dynamic light scattering
DOPA=dioleoyl-sn-glycero-3-phosphate
DOPC=1,2-dioleoyl-sn-glycero-3-phosphate sodium salt
DSPE-PEG₂ₖ=1,2-distearoyl-sn-glycero-3-phosphoethanolamine-N-[amino(polyethylene glycol)₂₀₀₀]
DTX=docetaxel
ET=etoposide
EtOAc=ethyl acetate
EtOH=ethanol
g=gram
GEM=gemcitabine
GMP=gemcitabine monophosphate
h=hour
IC₅₀=fifty percent inhibitory concentration
ICP-MS=inductively coupled plasma-mass spectrometry
IFN=interferon
IL=interleukin
i.v.=intravenous
Kₐ=association constant
kg=kilogram
LDL=low-density lipoprotein
M=molar
mCRC=metastatic colorectal cancer
mg=milligram
Mg=magnesium
min=minute
miRNA=microRNA
mL=milliliter
mm=millimeter
mM=millimolar
mmol=millimole
Mn=manganese
MOF=metal-organic framework
NCP=nanoscale coordination polymer
NIR=near infrared
nm=nanometer
NMR=nuclear magnetic resonance
OA=oleic acid
OxPt=oxaliplatin
PBS=phosphate buffered saline
PDI=polydispersity or polydispersity index
PD-I=programmed death 1
PD-L1=programmed death-ligand 1
PDT=photodynamic therapy
PEG=polyethylene glycol
P-gp=P-glycoprotein
PPX=podophyllotoxin
PS=photosensitizer
Pt=platinum
PTX=paclitaxel
PVP=polyvinylpyrrolidone
Q3D=once every three days
rpm=revolutions-per-minute
SBU=secondary building units
siRNA=small interfering RNA
SN38=7-ethyl-I0-hydroxycamptothecin
THF=tetrahydrofuran
TMS=trimethylsilyl
Zn=zinc

### BACKGROUND

The three main platinum drugs-cisplatin, oxaliplatin, and carboplatin—are alkylating agents which inhibit DNA replication, with nearly 50% of all tumor chemotherapy regimens including cisplatin (CisPt). Platinum-based doublet therapy is frequently used in the clinic for the treatment of ovarian, cervical, lung, and triple-negative breast cancer and has been further investigated in many cancers as first-line, second-line, or salvage therapies. These platinum drugs are often given in combination with topoisomerase inhibitors or mitotic inhibitors, such as paclitaxel (PTX). Combination therapy is often valuable due to the heterogeneity of cells in the tumor: some cells can be mitotically active while others are senescent, some cells can be resistant to one drug but not the other. For instance, oxaliplatin is used in combination with 5-fluorouracil (5-FU) and irinotecan for treating metastatic pancreatic cancer patients who have good performance status. Additional chemotherapy regimens containing multiple drugs include FOLFOX (folinic acid, fluorouracil, and oxaliplatin), FOLFIRI (folinic acid, fluorouracil, and irinotecan), and IROX (irinotecan and oxaliplatin). However, these treatments can have narrow therapeutic windows, sometimes with severe side effects. For example, 30% of metastatic colorectal cancer (mCRC) patients treated with the IROX regimen experienced severe neutropenia whereas 18% patients had severe sensory disturbances (Stanculeanu et al., Journal of Clinical Oncology 2006, 24:13541-13541). These side effects can be attributed to non-selective distribution of the drugs to bone marrow and peripheral nerves, respectively.

Nanoparticles can provide a platform for chemotherapy delivery by controlling physical properties, such as surface charge, to improve pharmacokinetic behavior and change the toxicity profile. PTX, a hydrophobic molecule, was originally formulated with Cremophor EL/ethanol to solubilize the drug in water solutions. However, the Cremophor EL formulation can lead to “severe anaphylactoid hypersensitivity reactions, hyperlipidaemia, abnormal lipoprotein patterns, aggregation of erythrocytes and peripheral neuropathy.” In 2005, a solvent-free albumin-bound paclitaxel (nab-paclitaxel) nanoparticle was approved in the United States and shifted the primary toxicity to neutropenia, the severity of which is correlated to the peak and sustained levels of free drug circulating in the bloodstream. Currently, however, there are no FDA-approved methods to deliver multiple chemotherapeutics of different physicochemical properties in a single nanoparticle for combination therapy.

Accordingly, there is an ongoing need for additional compositions and methods of delivering chemotherapeutic agents, particularly for combinations of agents with different mechanisms of action and/or physicochemical properties. There is also an ongoing need to provide additional compositions and methods of treating cancer that provide improved anti-cancer activity at a lower dose of the active agent or agents and/or with reduced levels of side effects.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter provides a prodrug comprising a structure of the formula D-BL-L, wherein D is a monovalent drug moiety, optionally wherein D is a monovalent derivative of an anti-cancer drug compound, further optionally wherein D is a monovalent derivative of a drug compound selected from the group comprising Etoposide (ET), Podophyllotoxin (PPX), Paclitaxel (PTX), Docetaxel (DTX), dihydroartemisin (DHA), Camptothecin (CPT), 7-ethyl-10-hydroxycamptothecin (SN38), Topotecan, Doxorubicin, Epirubicin, Idarubicin, Vincristine, Mitoxantrone, Artesunate, Capecitabine, Octreotide, Leuprolide, and Goserelin; L is a monovalent lipid moiety; and BL is a bivalent linker, wherein D is directly attached to BL via a carbonate or carbamate group, and wherein BL comprises at least one of an acetal group and a substituted oxybenzyloxy group, wherein the acetal group has a structure of one of the formulas:

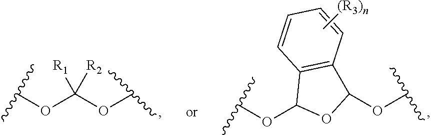

wherein: n is an integer between 0 and 4, optionally wherein n is 0; R₁ and R₂ are independently selected from the group comprising H, alkyl, substituted alkyl, aralkyl, substituted aralkyl, aryl, and substituted aryl, each R₃ is independently selected from the group comprising alkyl, aralkyl, aryl, a halo, alkoxy, aryloxy, hydroxy, acyl, carboxylate, phosphate, nitro, —N₃, B(OH)₂, and cyano; and wherein an oxygen atom of the acetal group is directly attached to a carbon atom of a carbonate or carbamate group; and wherein the substituted oxybenzyloxy group has a structure of the formula:

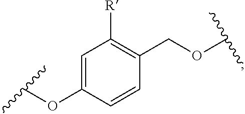

wherein R′ is selected from the group comprising nitro, N₃, and —B(OH)₂, and wherein the oxygen atom attached to the benzyl carbon of the oxybenzyloxy group is directly attached to a carbon atom of a carbonate or carbamate group.

In some embodiments, L is a monovalent derivative of cholesterol, oleic acid, a lyso-lipid, or phosphocholine. In some embodiments, the prodrug comprises a structure of one of formulas:

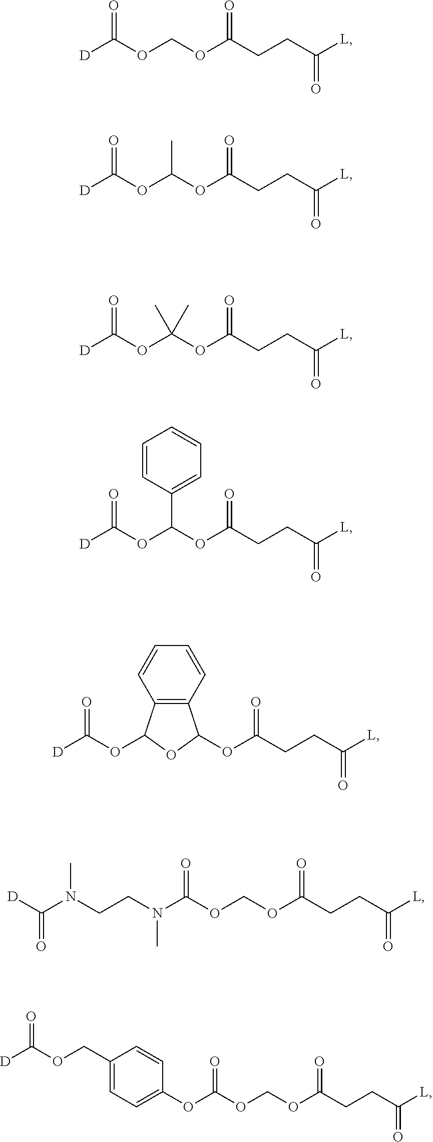

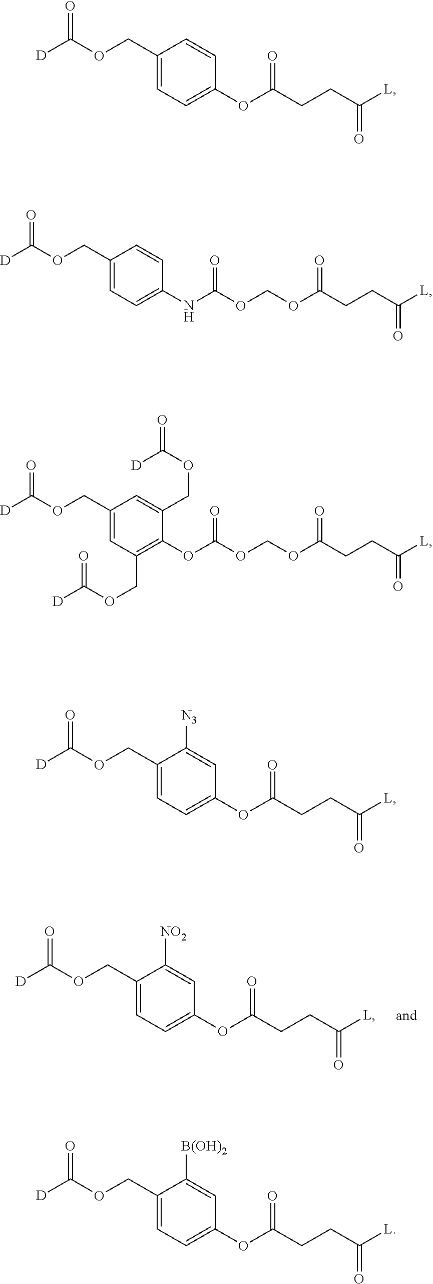

In some embodiments, BL comprises an acetal group having a structure of the formula:

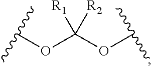

wherein R₁ and R₂ are independently selected from the group comprising H, alkyl, substituted alkyl, aralkyl, substituted aralkyl, aryl, and substituted aryl; optionally wherein R₁ and R₂ are independently selected from the group comprising H, methyl, and phenyl; further optionally wherein both R₁ and R₂ are H.

In some embodiments, L is an oleic acid moiety, and L and BL together have the structure:

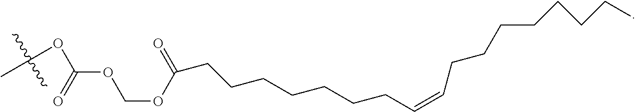

In some embodiments, L is a cholesterol derivative, and L and BL together have the structure:

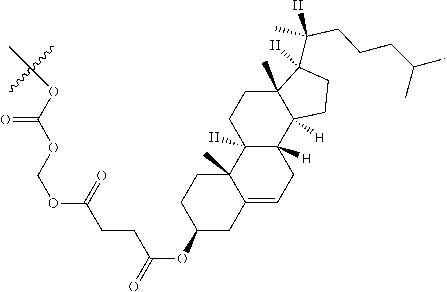

In some embodiments, the prodrug is selected from the group comprising:

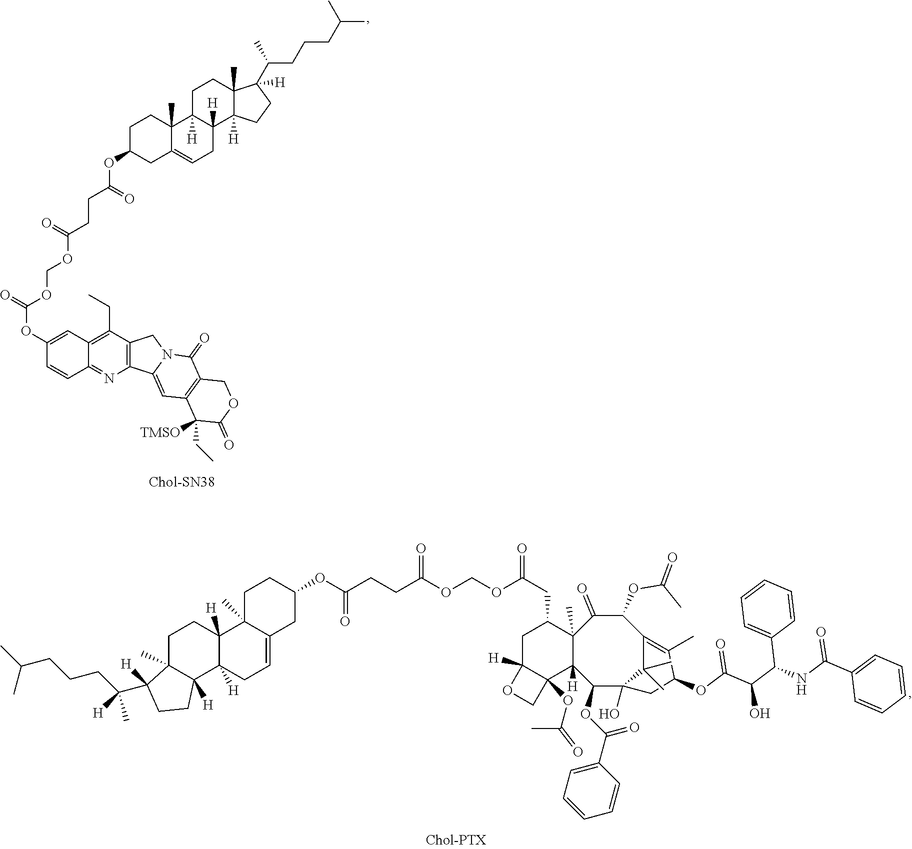

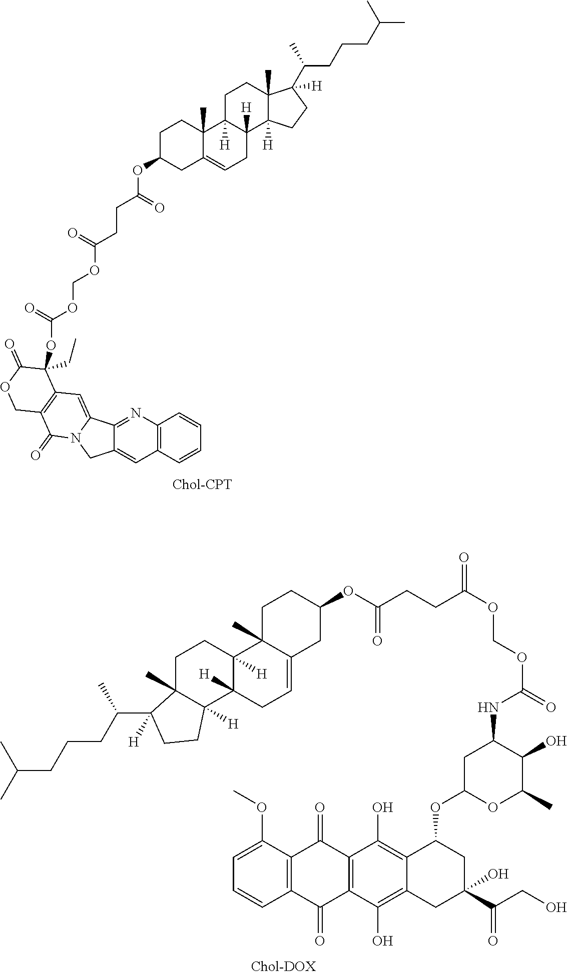

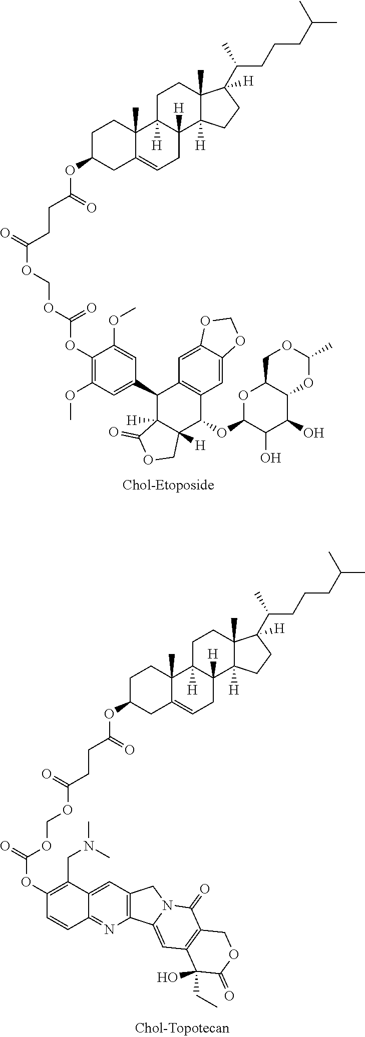

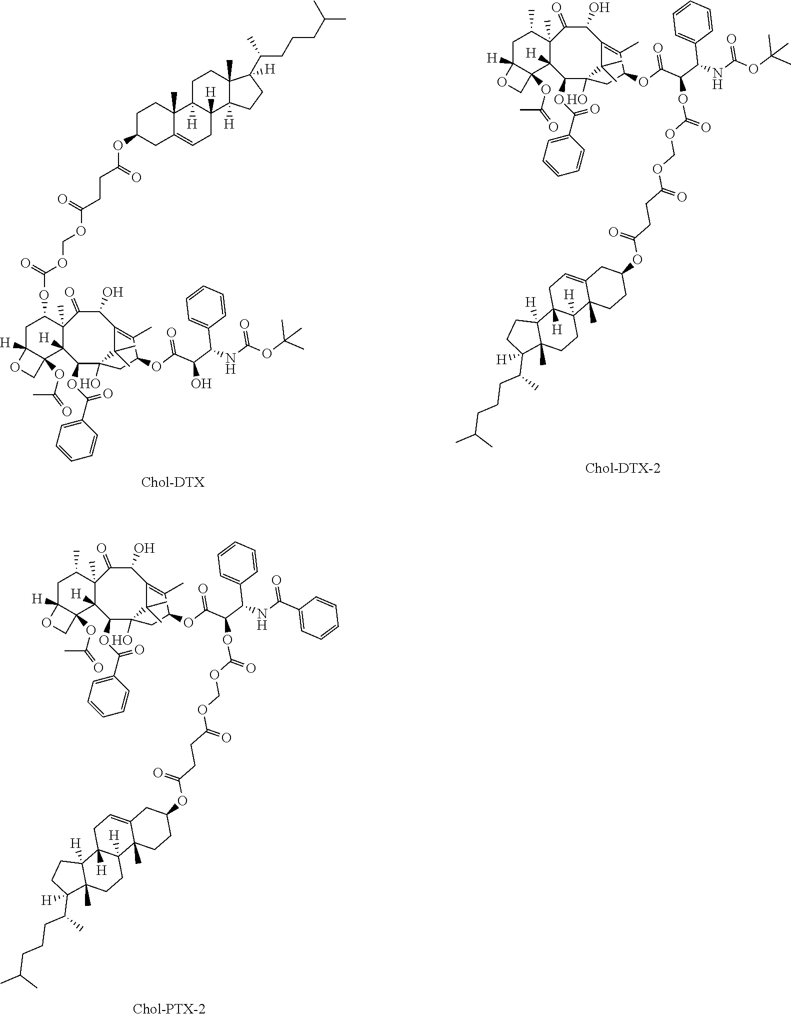

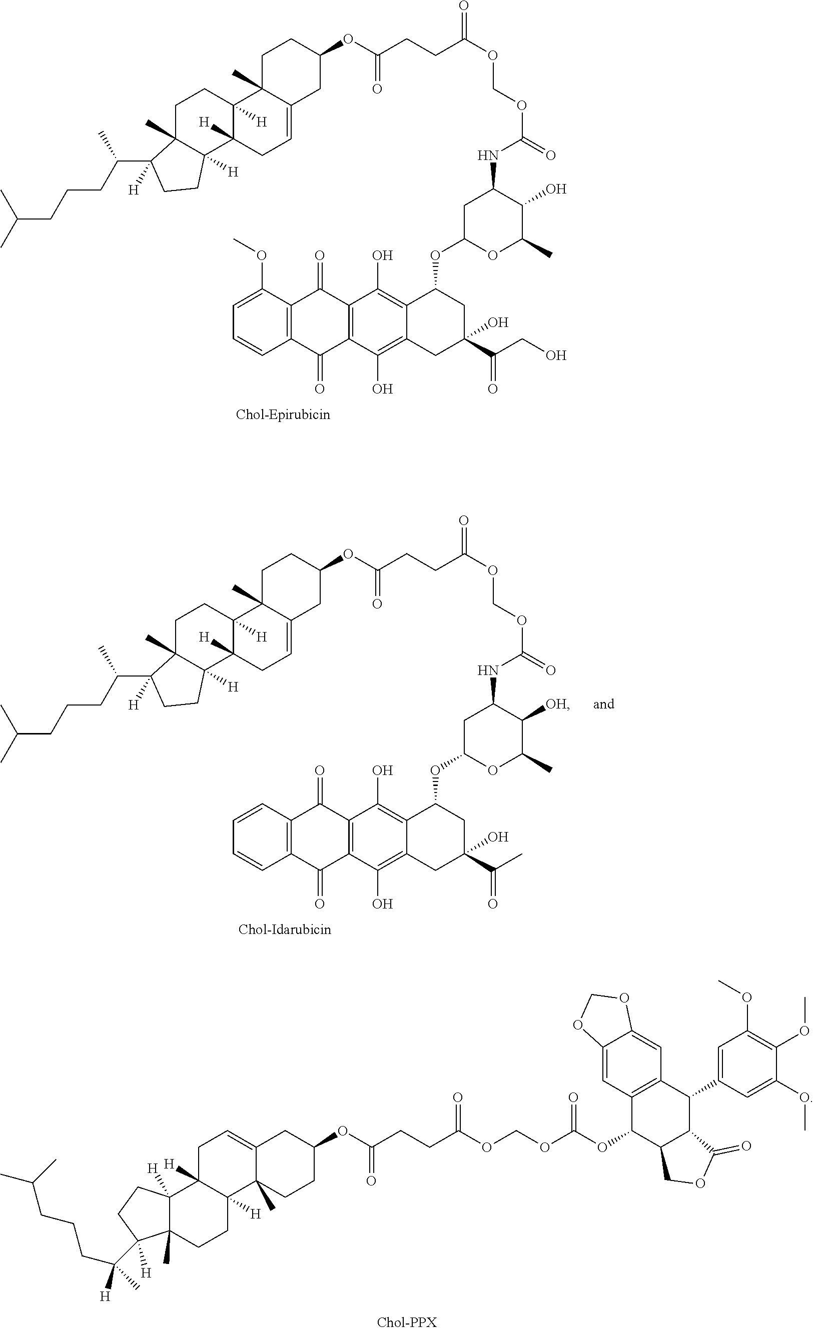

In some embodiments, the prodrug binds to low-density lipoprotein (LDL) and is actively transported to tumors via LDL-receptor mediated endocytosis, optionally wherein the prodrug has an association constant Kₐ for LDL that is at least about 1000 times the Kₐ of the prodrug for albumin, further optionally wherein the prodrug has a Kₐ for LDL that is at least about 2000 times that of the Kₐ of the prodrug for albumin.

In some embodiments, the presently disclosed subject matter provides a nanoparticle comprising: (a) a core comprising a metal-organic matrix material, optionally wherein the metal-organic matrix material comprises a coordination polymer; and (b) a coating layer covering at least a portion of the surface of the core, wherein said coating layer comprises a lipid layer or a lipid bilayer and wherein said coating layer comprises one or more prodrug comprising a structure of the formula D-BL-L. In some embodiments, the metal-organic matrix material comprises a nanoscale coordination polymer comprising a metal bisphosphate comprising a multivalent metal ion and a bisphosphate, optionally wherein the multivalent metal ion is selected from the group consisting of Ca²⁺, Mg²⁺, Mn²⁺, Zn²⁺ and combinations thereof. In some embodiments, the bisphosphate comprises a prodrug of an anti-cancer agent, optionally wherein the bisphosphate comprises a cisplatin, carboplatin or oxaliplatin prodrug, further optionally wherein the bisphosphate is a bisphosphate ester of cis, cis-trans-[Pt(NH₃)₂Cl₂(OH)₂] or cis, trans-[Pt(dach)(oxalate)(OH)₂].

In some embodiments, the core comprises an embedded anti-cancer agent, optionally an embedded hydrophilic anti-cancer agent, further optionally wherein the embedded anti-cancer agent is gemcitabine monophosphate (GMP). In some embodiments, the core comprises at least two anti-cancer agents, optionally wherein the at least two anti-cancer agents comprise a first anti-cancer agent, wherein the first anti-cancer agent is a cisplatin, carboplatin or oxaliplatin prodrug, further optionally a bisphosphate of cisplatin, carboplatin or oxaliplatin; and a second anti-cancer agent, wherein the second anti-cancer agent is an embedded, hydrophilic anti-cancer agent.

In some embodiments, the nanoparticle core comprises a metal bisphosphate coordination polymer comprising a multivalent metal ion, optionally Zn²⁺, and a bisphosphate, wherein said bisphosphate is an oxaliplatin prodrug having the structure Pt(dach)(oxalate)(bisphosphoramidic acid); and wherein the coating layer is a lipid bilayer comprising a prodrug having the structure:

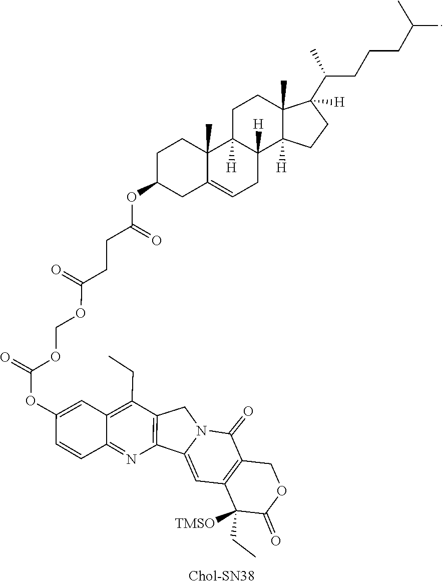

In some embodiments, the nanoparticle core further comprises GMP embedded in the nanoparticle core.

In some embodiments, the coating layer comprises a lipid bilayer comprising a cationic lipid and/or a functionalized lipid, wherein said functionalized lipid is a lipid functionalized with a group that can bond to a nucleic acid, and wherein at least one nucleic acid is covalently bonded to the functionalized lipid or attached to the cationic lipid via electrostatic interactions, optionally wherein said lipid bilayer comprises a mixture comprising one or more of a thiol- or dithiol-functionalized 1,2-distearoyl-sn-glycero-3-phosphoethanolamine (DSPE), 1,2-dioleoyl-3-trimethylammonium propane (DOTAP), and 1,2-dioleoyl-sn-glycero-3-phosphocholine (DOPC). In some embodiments, the at least one nucleic acid is selected from the group comprising a siRNA, a miRNA, and an AS ODN, optionally wherein the siRNA is selected from the group comprising survivin siRNA, ERCC-1 siRNA, P-glycoprotein siRNA (P-gp siRNA), Bcl-2 siRNA, and a mixture thereof.

In some embodiments, the nanoparticle further comprises one or more passivating agents, optionally a hydrophilic polymer; a targeting agent, optionally a RGD peptide; and an immunotherapy agent. In some embodiments, the nanoparticle has a diameter ranging from about 20 nanometers to about 140 nanometers. In some embodiments, the nanoparticle adsorbs plasma proteins, optionally apolipoprotein B-100, for active transport to tumors via LDL receptor-mediated endocytosis.

In some embodiments, the presently disclosed subject matter provides a pharmaceutical formulation comprising (i) a pharmaceutically acceptable carrier and (ii) a prodrug comprising a structure of the formula D-BL-L or a nanoparticle comprising (a) a core comprising a metal-organic matrix material, optionally wherein the metal-organic matrix material comprises a coordination polymer, and (b) a coating layer covering at least a portion of the surface of the core, wherein said coating layer comprises a lipid layer or a lipid bilayer and wherein said coating layer comprises one or more prodrug comprising a structure of the formula D-BL-L.

In some embodiments, the presently disclosed subject matter provides a method of treating cancer in a subject in need thereof, wherein the method comprises administering to the subject a prodrug comprising a structure of the formula D-BL-L; a nanoparticle comprising: (a) a core comprising a metal-organic matrix material, optionally wherein the metal-organic matrix material comprises a coordination polymer, and (b) a coating layer covering at least a portion of the surface of the core, wherein said coating layer comprises a lipid layer or a lipid bilayer and wherein said coating layer comprises one or more prodrug comprising a structure of the formula D-BL-L; or a pharmaceutical formulation thereof.

In some embodiments, the method comprises administering to the subject an additional cancer treatment selected from the group comprising surgery, radiotherapy, chemotherapy, toxin therapy, immunotherapy, cryotherapy and gene therapy; optionally wherein the additional cancer treatment is immunotherapy. In some embodiments, the immunotherapy comprises administering to the subject an immunotherapy agent; optionally wherein the immunotherapy agent is selected from the group comprising an anti-CD52 antibody, an anti-CD20 antibody, an anti-CD47 antibody an anti-GD2 antibody, a cytokine, polysaccharide K; a PD-1 inhibitor, a PD-L1 inhibitor, a CTLA-4 inhibitor, an IDO inhibitor, a CCR7 inhibitor, an OX40 inhibitor, a TIM3 inhibitor, and a LAG3 inhibitor.

In some embodiments, the cancer is selected from the group comprising a head tumor, a neck tumor, breast cancer, a gynecological tumor, a brain tumor, colorectal cancer, lung cancer, mesothelioma, a soft tissue sarcoma, skin cancer, connective tissue cancer, adipose cancer, lung cancer, stomach cancer, anogenital cancer, kidney cancer, bladder cancer, colon cancer, prostate cancer, central nervous system cancer, retinal cancer, blood cancer, neuroblastoma, multiple myeloma, lymphoid cancer, and pancreatic cancer. In some embodiments, the cancer is a metastatic cancer, optionally a metastatic colorectal cancer.

In some embodiments, the method comprises administering to the subject a nanoparticle wherein the nanoparticle core comprises a metal bisphosphate coordination polymer comprising a multivalent metal ion, optionally selected from Ca²⁺, Mg²⁺, Mn²⁺, Zn²⁺ and combinations thereof, and a bisphosphate, wherein said bisphosphate is a bisphosphate ester of cisplatin, oxaliplatin or carboplatin; and wherein the coating layer comprises a lipid bilayer comprising a prodrug having the structure D-BL-L wherein D is a monovalent drug moiety of an anti-cancer drug compound, optionally wherein the monovalent drug moiety is a monovalent derivative of a drug compound selected from the group comprising ET, PPX, PTX, DTX, DHA, CPT, SN38, Topotecan, Doxorubicin, Epirubicin, Idarubicin, Vincristine, Mitoxantrone, Artesunate, Capecitabine, Octreotide, Leuprolide, and Goserelin; L is a monovalent lipid moiety, optionally a monovalent cholesterol moiety; and BL is a bivalent linker moiety wherein D is attached to BL via a carbonate or a carbamate bond, and wherein BL comprises an acetal group, wherein the acetal group has a structure of the formula:

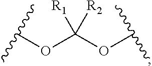

wherein R₁ and R₂ are independently selected from the group comprising H, alkyl, substituted alkyl, aralkyl, substituted aralkyl, aryl, and substituted aryl; and wherein at least one of the oxygen atoms in the acetal group is directly bonded to a carbon atom of a carbonate or carbamate group. In some embodiments, the nanoparticle core further comprises a hydrophilic anti-cancer agent embedded therein, optionally wherein the hydrophilic anti-cancer agent is GMP. In some embodiments, prodrug has a structure of the formula:

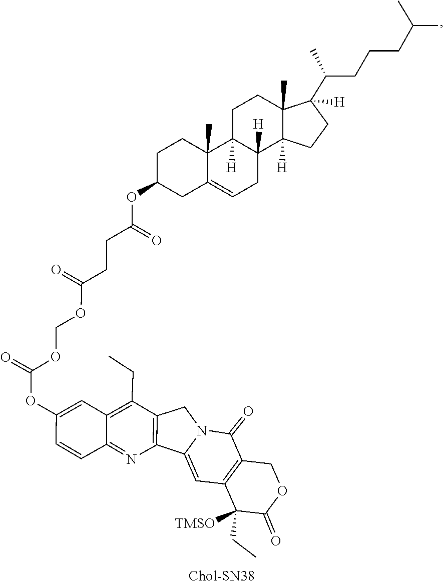

optionally wherein the bisphosphate is Pt(dach)(oxalate)(bisphosphoramidic acid).

In some embodiments, the method further comprises administering to the subject an immunotherapy agent. In some embodiments, administration of the nanoparticle provides at least a 2-fold increase, optionally a greater than 4-fold increase, in a tumor area under the curve (AUC) of at least one anti-cancer agent compared to administration of an equivalent amount of the at least one anti-cancer agent wherein the at least one anti-cancer agent is not associated with a nanoparticle and/or prodrug.

Accordingly, it is an object of the presently disclosed subject matter to provide prodrugs comprising linkers comprising an acetal or an oxybenzyloxy group attached to a carbonate or carbamate, nanoparticles comprising the prodrugs, and formulations of the prodrugs and nanoparticles, as well as methods of treating cancer using the prodrugs, nanoparticles, and formulations.

An object of the presently disclosed subject matter having been stated hereinabove, and which is achieved in whole or in part by the presently disclosed subject matter, other objects will become evident as the description proceeds hereinbelow.

### BRIEF DESCRIPTION OF THE DRAWINGS

FIG. 1 is a synthetic diagram showing a synthetic route to an exemplary lipid prodrug of the presently disclosed subject matter, i.e., a prodrug referred to herein as “Chol-SN38”, which comprises a monovalent lipid moiety based on cholesterol and a monovalent drug moiety based on 7-ethyl-10-hydroxycamptothecin (SN38) which further comprises a trimethylsilyl (TMS) ether.

FIG. 2 is a schematic diagram showing a two-step construction of an exemplary core-shell nanoparticle of the presently disclosed subject matter (referred to herein as “OxPt/SN38”). The nanoparticle (NP) core comprises a metal-bisphosphate coordination polymer prepared by the copolymerization of zinc ions (Zn²⁺) and a bisphosphoramidic acid derivative of oxaliplatin (i.e., OxPt-bp). The NP coating layer comprises the lipid prodrug of 7-ethyl-10-hydroxycamptothecin (SN38), i.e., Chol-SN38, described in FIG. 1, in addition to cholesterol, 1,2-dioleoyl-sn-glycero-3-phosphate sodium salt (DOPC) and 1,2-distearoyl-sn-glycero-3-phosphoethanolamine-N-[amino(polyethylene glycol)₂₀₀₀] (DSPE-PEG₂₀₀₀).

FIGS. 3A-3C are a microscopy image and a pair of graphs related to the characterization of nanoparticles comprising a zinc/oxaliplatin bisphosphate coordination polymer core without a lipid bilayer coating, i.e., “OxPt-bare.” FIG. 3A is a transmission electron microscopy (TEM) image of OxPt-bare nanoparticles. The scale bar in the lower left corner represents 50 nanometers (nm). FIG. 3B is a graph showing the number-average diameter of OxPt-bare nanoparticles measured by dynamic light scattering (DLS). The diameter is measured in nanometers (nm). FIG. 3C is a graph showing the stability of the OxPt-bare nanoparticles in tetrahydrofuran (THF) at room temperature over time. The solid line shows data for the number-average nanoparticle diameter (in nm) of the nanoparticle versus time (in hours), while the dotted line shows polydispersity (PDI) versus time.

FIGS. 4A-4C are a microscopy image and a pair of graphs related to the characterization of nanoparticles comprising a zinc/oxaliplatin bisphosphate coordination polymer core and having a lipid bilayer coating comprising the lipid prodrug of 7-ethyl-10-hydroxycamptothecin (SN38) described in FIG. 1, i.e., OxPt/SN38 nanoparticles. FIG. 4A is a transmission electron microscopy (TEM) image of OxPt/SN38 nanoparticles. The scale bar in the lower left represents 100 nanometers. FIG. 4B is a graph showing the number-average diameter of OxPt/SN38 nanoparticles measured by dynamic light scattering (DLS). The diameter is measured in nanometers (nm). FIG. 4C is a graph showing the stability of the OxPt/SN38 nanoparticles in phosphate buffered saline (PBS) with bovine serum albumin (BSA) at 5 milligrams per milliliter (mg/mL) at 37° C. The solid line shows data for the number-average nanoparticle diameter (in nm) of the nanoparticles versus time (in hours), while the dotted line shows polydispersity (PDI) versus time.

FIGS. 5A-5C are a series of graphs showing that the nanoparticles comprising a core comprising an oxaliplatin (OxPt) prodrug (i.e., an oxaliplatin bisphosphate) and a lipid layer comprising a cholesterol prodrug of 7-ethyl-10-hydroxycamptothecin (Chol-SN38), i.e., OxPt/SN38, transfer Chol-SN38 to low-density lipoprotein (LDL). FIG. 5A is a graph showing the potentials of mean force (PMF) (measured in kilojoules per mole (kJ-mol-) of transferring free drug, i.e., 7-ethyl-10-hydroxycamptothecin (SN38, solid line) and prodrug (Chol-SN38, dashed line) from bulk water to the lipid core of an LDL slice from molecular dynamics (MD) simulations. The plots are superimposed onto a snapshot of an equilibrated LDL slice. FIG. 5B is a graph showing the time-dependent binding (measured as a percentage (%)) of SN38 (dotted line) or Chol-SN38 (solid line) to LDL and transfer of chol-SN38 from OxPt/SN38 to LDL in rat plasma. FIG. 5C is a pharmacokinetic profile of Chol-SN38 from OxPt/SN38 in rat plasma and its lipoprotein distribution (albumin (white), high-density lipoprotein (HDL, light grey), LDL (dark grey), or very low-density lipoprotein (VLDL, black)) after intravenous injection of OxPt/SN38 at a Chol-SN38 dose of 14.4 milligrams per kilogram (mg/kg). Chol-SN38 is measured in micrograms per milliliter (g/mL) at 0.5, 1, 3, 5, 8, and 24 hours.

FIG. 6 is a graph showing concentration-dependent apolipoprotein B-100 (ApoB-100) binding to a zinc pyrophosphate nanoscale coordination polymer (ZnP NCP). Data is shown for ZnP NCP (solid line) and ZnP NCP without cholesterol (dashed line), n=3. ZnP NCP concentration is shown on the x axis measured in milligram (mg).

FIGS. 7A-7C are graphs showing uptake of nanoparticles via the low-density lipoprotein receptor (LDLR). FIG. 7A is a graph showing uptake of a nanoscale coordination polymer particle comprising a lipid coating layer comprising a cholesterol-pyrolipid conjugate (Chol-pyro NCP) or a fluorescently labeled low-density lipoprotein (Dil-LDL) and FIG. 7B is a graph showing uptake of a nanoscale coordination polymer particle comprising a core comprising chlorin e6 (Ce6-NCP) or Dil-LDL by murine colon adenocarcinoma (MC38) cells after LDLR blockade with 1 or 10 microgram per milliliter (μg/ml) anti-LDLR antibody (a-LDLR). FIG. 7C is a graph showing cellular uptake of Chol-pyro NCP and Ce6-NCP on wildtype (WT) and LDLR knockout (KO) MC38 cells. Cellular uptake is measured via measurement of relative mean fluorescence intensity (MFI), reported as a percentage compared to control.

FIGS. 8A and 8B are a pair of graphs where FIG. 8A is a graph showing the confocal laser scanning microscopy (CLSM) statistical analysis of uptake of a cholesterol conjugate of pyrolipid (Chol-pyro) from a nanoscale coordination polymer (NCP) comprising a lipid layer comprising the conjugate (Chol-pyro NCP) by murine colon adenocarcinoma (MC38) cells 24 hours after treatment with 10 micrograms per milliliter (μg/ml) a non-specific immunoglobulin G (IgG) or an anti-low-density lipoprotein receptor antibody (a-LDLR), while FIG. 8B is a graph of the mean fluorescent intensity (MFI) of tumor uptake of Chol-pyro NCP at 24 hours (h) and 48 h post intravenous (i.v.) injection with 1 microgram (g) of IgG or a-LDLR.

FIGS. 9A and 9B are graphs showing the time dependent accumulation (presented as micromolar (M) concentration) of platinum (Pt) (FIG. 9A) and 7-ethyl-10-hydroxycamptothecin (SN38) (FIG. 9B) after intravenous (i.v.) injection of free oxaliplatin (OxPt, 3.5 milligrams per kilogram (mg/kg)) plus irinotecan (6.2 mg SN38/kg equivalent) or a nanoparticle comprising a core comprising oxaliplatin (OxPt) prodrug and a lipid layer comprising a lipid prodrug of SN38 (OxPt/SN38; 3.5 mg OxPt/kg equivalent, 6.2 mg SN38/kg equivalent) in murine colon adenocarcinoma (MC38)-bearing mice with and without 1 microgram (μg) of intratumorally injected anti-low-density lipoprotein receptor antibody (a-LDLR).

FIGS. 10A-10C. FIG. 10A is a series of graphs showing apoptosis induced by free oxaliplatin (OxPt) plus irinotecan or a nanoparticle comprising a core comprising OxPt prodrug and a lipid coating layer comprising a lipid prodrug of 7-hydroxy-10-ethylcamptothecin (Sn38), i.e., OxPt/SN38. After treatment, cells were stained by Alexa Fluor 488-labelled Annexin V and propidium iodide (PI) and analyzed by flow cytometry. FIG. 10B is a graph showing cell cycle arrest caused by OxPt/SN38. Treated cells were fixed with 70% ethanol overnight, treated with RNase A, stained by PI, and analysed by flow cytometry, n=3. FIG. 10C is a graph showing flow cytometry analysis (presented as mean fluorescence intensity (MFI)) with JC-1 staining of mitochondrial membrane potentials of MC38 cells treated with free OxPt plus irinotecan or OxPt/SN38, n=3.

FIGS. 11A and 11B are a pair of graphs showing the percentages (% s) of cholesterol-7-ethyl-10-hydroxycamptothecin prodrug (Chol-SN38) and released products (free 7-ethyl-10-hydroxycamptothecin (SN38), Chol-SN38 with no trimethylsilyl (TMS) ether (Chol-SN38 (No TMS)) or SN38 with TMS ether (SN38-TMS)) from nanoparticles comprising a core comprising oxaliplatin prodrug and a lipid coating layer comprising the prodrug (OxPt/SN38) at pH=4.7 in phosphate buffered saline (PBS) (FIG. 11A) and pH=7.4 PBS with 10 unit/milliliter (mL) esterase (FIG. 11B) throughout 72 hours at 37 degrees Celsius (° C.).

FIGS. 12A and 12B are graphs showing the cumulative release (measured as a percentage (%)) of 7-ethyl-10-hydroxycamptothecin (SN38) (FIG. 12A) and platinum (Pt) (FIG. 12B) from a nanoparticle comprising a core comprising oxaliplatin prodrug and a lipid layer comprising a SN38-based lipid prodrug (OxPt/SN38) when incubated in phosphate buffered saline (PBS) at pH=4.7 (dashed lines) or pH=7.4 (solid lines) throughout 72 hours at 37 degrees Celsius (° C.).

FIG. 13 is a schematic diagram of proposed 7-ethyl-10-hydroxycamptothecin (SN38) release mechanisms from a prodrug of the presently disclosed subject matter via acid-catalyzed hydrolysis and esterase-mediated cleavage.

FIGS. 14A and 14B are (FIG. 14A) a schematic diagram showing the release of oxaliplatin (OxPt) from a nanoscale coordination polymer (NCP) of zinc and an OxPt bisphosphate prodrug via hydrolysis to provide an oxaliplatin biscarbamate (OxPt-bc) followed by reduction by ascorbate; and (FIG. 14B) a graph showing total platinum (Pt) OxPt, and OxPt-bc release profiles from a nanoparticle comprising a core comprising the NCP described for FIG. 14A and a lipid layer comprising a lipid prodrug of 7-ethyl-10-hydroxycamptothecin (OxPt/SN38) when incubated in phosphate buffered saline (PBS) with pH=4.7 and 5 millimolar (mM) ascorbate at 37 degrees Celsius (° C.).

FIGS. 15A and 15B are a pair of graphs showing (FIG. 15A) total plasma platinum (Pt) concentration (measured in micrograms per milliliter (μg/ml) over time (in hours); and (FIG. 15B) plasma concentrations (measured in g/ml) of 7-ethyl-10-hydroxycamptothecin (SN38, dotted line), 20-O-trimethylsilyl-SN38 (SN38-TMS, dashed line), and a cholesterol prodrug of SN38 (Chol-SN38, solid line) over time (in hours) in mice dosed with 2 milligrams per kilogram (mg/kg) of a nanoparticle comprising an oxaliplatin (OxPt) prodrug core and a coating layer comprising Chol-SN38.

FIGS. 16A and 16B are a pair of graphs showing tumor (FIG. 16A) and plasma (FIG. 16B) 7-ethyl-10-hydroxycamptothecin (SN38) concentrations (measured in micrograms per milliliter (μg/ml)) over time (measured in hours (h)) in colorectal carcinoma (CT26) tumor-bearing mice intravenously (i.v.) injected with a nanoparticle comprising an oxaliplatin (OxPt) prodrug-containing core and a lipid coating layer comprising a lipid prodrug of SN38 at a dose of nanoparticle equivalent to 3 milligrams per kilogram (mg/kg) body weight OxPt.

FIG. 17 is a graph of the body weight (measured as a percentage (%) of body weight on the first day of dosing) of Balb/c mice after repeated doses of a nanoparticle comprising an oxaliplatin (OxPt) prodrug-containing core and a lipid coating layer comprising a lipid prodrug of 7-ethyl-10-hydroxycamptothecin (SN38). Mice were dosed on a once every three day (Q3D) schedule and at a dose of nanoparticle equivalent to 3 milligrams per kilogram (mg/kg) body weight OxPt.

FIG. 18 is a graph showing the tumor growth inhibition of murine adenocarcinoma (MC38) tumors in tumor-bearing mice after various treatments dosed on a once every three day (Q3D) schedule. Treatments included: phosphate buffered saline (PBS, squares) as a control, a mixture of free oxaliplatin (OxPt) and irinotecan (OxPt+Irinotecan, triangles pointing up), a nanoparticle with a core comprising an OxPt prodrug (OxPt NCP, triangles pointing down), a nanoparticle with a zinc pyrophosphate (ZnP) coordination polymer core and a coating layer comprising a lipid prodrug of 7-ethyl-10-hydroxycamptothecin (ZnP/SN38, diamonds), a nanoparticle with a core comprising OxPt prodrug and a coating layer comprising a lipid prodrug of 7-ethyl-10-hydroxycamptothecin (OxPt/SN38, triangles pointing left), or OxPt/SN38 and an anti-programmed death ligand 1 antibody (OxPt/SN38+α-PD-L1, triangles pointing right). Treatments were given once every three days at an OxPt equivalent dose of 3 milligrams per kilogram (mg/kg) body weight. Tumor size was measured in cubic millimeters (mm³) on days 0-18 of treatment, starting with day 0 as the day of the first injection.

FIG. 19 is a graph showing tumor growth inhibition/regression of murine adenocarcinoma (MC38) tumors in tumor bearing mice after repeated doses of a nanoparticle comprising an oxaliplatin (OxPt) prodrug-containing core and a lipid coating layer comprising a lipid prodrug of 7-ethyl-10-hydroxycamptothecin (SN38) on a once every three day (Q3D, triangles pointing up) or a weekly/once every seven day (Q7D, triangles pointing down) schedule. Each dose for the Q3D schedule was at an OxPt equivalent dose of 3 milligrams per kilogram (mg/kg) body weight, while each dose on the Q7D schedule was at an OxPt equivalent dose of 6 mg/kg body weight. Phosphate buffered saline (PBS, squares) was used as a control. Tumor size was measured in cubic millimeters (mm³) on days 0-29 of treatment, starting with day 0 as the day of the first injection.

FIGS. 20A and 20B are a pair of graphs showing tumor growth inhibition/regression of murine colorectal carcinoma (CT26) tumors (FIG. 20A) and human colorectal adenocarcinoma (HT29) tumors (FIG. 20B) in tumor bearing mice after repeated doses of a nanoparticle comprising an oxaliplatin (OxPt) prodrug-containing core and a lipid coating layer comprising a lipid prodrug of 7-ethyl-10-hydroxycamptothecin (SN38) on a once every three day (Q3D, triangles pointing up) or a weekly/once every seven day (Q7D, triangles pointing down) schedule. Each dose for the Q3D schedule was at an OxPt equivalent dose of 3 milligrams per kilogram (mg/kg) body weight, while each dose on the Q7D schedule was at an OxPt equivalent dose of 6 mg/kg body weight. Phosphate buffered saline (PBS, squares) was used as a control. Tumor size was measured in cubic millimeters (mm³) on days 0-20 or days 0-21 of treatment, starting with day 0 as the day of the first injection.

FIG. 21 is a graph showing absolute neutrophil counts (measured in thousands of cells per microliter (10³ cells/μL) for murine colon adenocarcinoma (MC38) tumor-bearing C57BL/6 mice after three once every three day (Q3D) doses of free oxaliplatin (OxPt; 3.5 milligrams per kilogram (mg/kg)) plus irinotecan (11.7 mg/kg 7-ethyl-10-hydroxycamptothecin (SN38) equivalent) or eight Q3D doses of a nanoparticle comprising a core comprising an OxPt prodrug and a lipid layer comprising a lipid prodrug of SN38 (OxPt/SN38; 3.5 mg/kg OxPt equivalent and 6.2 mg/kg SN38 equivalent). For comparison, neutrophil counts are also provided for a control group treated with phosphate buffered saline (PBS).

FIGS. 22A and 22B are graphs showing tumor growth curves (FIG. 22A) and survival curves (FIG. 22B) in a human colorectal adenocarcinoma (HT29) model on nude mice after once every three day (Q3D) treatment with phosphate buffered saline (PBS, squares), a nanoparticle comprising a core comprising oxaliplatin (OxPt) prodrug and a lipid coating layer comprising a lipid prodrug of 7-ethyl-10-hydroxycamptothecin (OxPt/SN38, triangles pointing down), or free OxPt plus irinotecan (triangles pointing up) for up to 16 doses. Tumor size is measured in cubic centimeters (cm³) and survival as a percentage (%).

FIGS. 23A and 23B are graphs showing tumor growth curves of human colorectal adenocarcinoma (HCT116) (FIG. 23A) and SW480 (FIG. 23B) models on nude mice after once every three day (Q3D) treatment with phosphate buffered saline (PBS, squares), a nanoparticle comprising a core comprising oxaliplatin (OxPt) prodrug and a lipid coating layer comprising a lipid prodrug of 7-ethyl-10-hydroxycamptothecin (OxPt/SN38, triangles pointing down), or free OxPt plus irinotecan (triangles pointing up) for up to 16 doses. Tumor size is measured in cubic centimeters (cm³).

FIGS. 24A and 24B are graphs showing (FIG. 24A) anticancer efficacy of a nanoparticle comprising a core comprising oxaliplatin (OxPt) prodrug and a lipid coating layer comprising a lipid prodrug of 7-ethyl-10-hydroxycamptothecin (OxPt/SN38) with intratumorally injected 1 microgram (μg) non-specific immunoglobulin G (IgG, triangle pointing left) or an anti-low-density lipoprotein receptor antibody (anti-LDLR, triangles pointing down) on murine colorectal adenocarcinoma (MC38) tumor-bearing C57BL/6 mice at a dose of 3.5 milligrams (mg) OxPt/kilogram (kg) equivalent (n=6); and (FIG. 24B tumor weights (in grams (g)) of excised MC38 tumors on day 22, n=6. In FIG. 24A, data for mice treated with phosphate buffered saline (PBS) and IgG is shown in squares and data for mice treated with PBS and anti-LDLR is shown in triangles pointing up, while tumor size is measured in cubic centimeters (cm³).

FIG. 25 is a graph showing anticancer efficacy of a nanoparticle comprising a core comprising oxaliplatin (OxPt) prodrug and a lipid coating layer comprising a lipid prodrug of 7-ethyl-10-hydroxycamptothecin (SN38) (i.e., OxPt/SN38) on wild-type (WT) and low-density lipoprotein receptor (LDLR) knockout (KO) murine adenocarcinoma (MC38) tumor-bearing C57BL/6 mice at a dose of 3.5 milligrams (mg) OxPt per kilogram (kg) equivalent. n=6. The graph shows tumor size (in cubic centimeters (cm³)) versus days after the first injection. Data is shown for LDLR KO mice treated with PBS (LDLR KO PBS, squares) or with nanoparticle (LDLR KO OxPt/SN38, triangles pointing up), WT mice treated with PBS (triangles pointing down) and WT mice treated with nanoparticle (WT OxPt/SN38, triangles pointing left).

FIG. 26 is a graph showing the results of dynamic light scattering (DLS) measurement of a nanoparticle comprising a carboplatin (Carbo) prodrug-containing core and a lipid coating layer comprising a cholesterol prodrug of podophyllotoxin (PPX). Nanoparticle diameter is measured in nanometers (nm).

FIGS. 27A and 27B are a pair of graphs of tumor growth inhibition of murine colorectal carcinoma (CT26) tumors (FIG. 27A) in tumor-bearing mice and mouse body weight (FIG. 27B) after repeated doses of a nanoparticle comprising a core comprising oxaliplatin (OxPt) prodrug and gemcitabine (GEM) and a lipid coating layer comprising a disulfide linker-containing lipid prodrug of 7-ethyl-10-hydroxycamptothecin (SN38), i.e., OxPt/GEM/SN38(Boc) (arrows pointing left), or other treatments (i.e., phosphate buffered saline (PBS, squares), the same nanoparticle without a lipid prodrug (OxPt/GEM, triangles pointing up), and the same nanoparticle without GEM (OxPt/SN38(Boc), triangles pointing down)) on a once every three day (Q3D) schedule. Tumor size in FIG. 27A is measured in cubic centimeters (cm³) and body weight in FIG. 27B is measured as a percentage (%) of body weight on day 0 (the first day of treatment).

FIGS. 28A and 28B are a pair of graphs showing the body weights (FIG. 28A) and tumor growth curves (FIG. 28B) of KPC tumor-bearing C57bl/6 mice after treatment with phosphate buffered saline (PBS, squares), a nanoparticle with an oxaliplatin prodrug-containing core and a lipid prodrug of 7-ethyl-10-hydroxycamptothecin of the presently disclosed subject matter (OxPt/SN38, triangles pointing up), or the same nanoparticle further comprising gemcitabine (GEM) embedded in the nanoparticle core (OxPt/GEM/SN38, triangles pointing down) on a once every three day (Q3D) schedule. The KPC tumor is a model of human pancreatic ductal adenocarcinoma. Tumor size in FIG. 28B is measured in cubic centimeters (cm³) and body weight in FIG. 28A is measured as a percentage of body weight on day 0 (the first day of treatment).

FIGS. 29A and 29B are a pair of graphs showing the body weights (FIG. 29A) and tumor growth curves (FIG. 29B) of murine breast cancer (4T1) tumor-bearing balb/c mice treated with phosphate buffered saline (PBS, circles), free carboplatin (Carb) plus docetaxel (DTX) (triangles pointing down), a nanoparticle comprising a zinc pyrophosphate coordination polymer core with a lipid coating layer comprising a lipid prodrug of DTX (ZnP/DTX, squares), or a nanoparticle comprising a core comprising a carboplatin prodrug and a lipid coating layer comprising a lipid prodrug of DTX (Carb/DTX, diamonds) at an equivalent Carb dose of 5 milligrams per kilogram (mg/kg) once every week for 3 doses. Tumor volume in FIG. 29B is measured in cubic centimeters (cm³) and body weight in FIG. 29A is measured as a percentage (%) of body weight on day 0 (the first day of treatment).

FIGS. 30A and 30B are a pair of graphs showing the body weights (FIG. 30A) and tumor growth curves (FIG. 30B) of human non-small cell lung cancer (H460) tumor-bearing athymic nude mice treated with phosphate buffered saline (PBS, circles) or a nanoparticle comprising a core comprising carboplatin prodrug and a lipid coating layer comprising a lipid prodrug of docetaxel (Carb/DTX, diamonds) at an equivalent carboplatin dose of 5 milligrams per kilogram (mg/kg) once every week for 3 doses. Tumor volume in FIG. 30B is measured in cubic centimeters (cm³) and body weight in FIG. 30A is measured as a percentage (%) of body weight on day 0 (the first day of treatment).

FIGS. 31A and 31B are a pair of graphs showing plasma concentrations of a cholesterol prodrug of 7-ethyl-10-hydroxycamptothecin (SN38) with no trimethylsilyl (TMS) ether group (Chol-SN38 (NO TMS)) (FIG. 31A) and of SN38 (FIG. 31B) over time (in hours) following intravenous administration of a nanoparticle comprising a core comprising oxaliplatin (OxPt) and a lipid coating layer comprising Chol-SN38 (No TMS) to rats at a dose level of 2 milligrams OxPt per kilogram body weight. Plasma concentrations are measured in micrograms per milliliter (μg/ml)

FIGS. 32A and 32B are a pair of graphs showing the body weights (FIG. 32A) and tumor growth curves (FIG. 32B) of human colorectal adenocarcinoma (HT29) tumor-bearing nude mice after various treatments (phosphate buffered saline (PBS, squares), a mixture of free oxaliplatin (OxPt) and irinotecan (triangles pointing up), or a nanoparticle comprising a core comprising OxPt prodrug and a lipid coating layer comprising a cholesterol prodrug of 7-ethyl-10-hydroxycamptothecin (SN38) with no trimethylsilyl (TMS) group (OxPt/SN38 (No TMS), triangles pointing down) on a once every three day (Q3D) schedule for 16 doses, n=6. Tumor size in FIG. 32B is measured in cubic centimeters (cm³) and body weight in FIG. 32A is measured as a percentage (%) of body weight on day 0 (the first day of treatment).

FIG. 33 is a schematic drawing showing the chemical structures of several exemplary cholesterol (Chol) prodrugs of the presently disclosed subject matter.

FIG. 34 is a schematic drawing showing the chemical structures of several additional exemplary cholesterol (Chol) prodrugs of the presently disclosed subject matter.

FIG. 35A is a schematic drawing showing general chemical structures of several exemplary prodrugs of the presently disclosed subject matter, wherein the exemplary prodrugs have various bivalent linker structures.

FIG. 35B is a schematic drawing showing the chemical structures of (i) a monovalent cholesterol moiety that can be used as “Chol” in the exemplary prodrugs in FIG. 35A, and (ii) monovalent drug moieties that can be used as “Drug” in the exemplary prodrugs in FIG. 35A.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a metal ion” includes a plurality of such metal ions, and so forth.

Unless otherwise indicated, all numbers expressing quantities of size, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of size (i.e., diameter), weight, concentration or percentage is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g. 1 to 5 includes, but is not limited to, 1, 1.5, 2, 2.75, 3, 3.90, 4, and 5).

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct or method within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein the term “alkyl” can refer to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. In some embodiments, there can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Heteroaryl” as used herein refers to an aryl group that contains one or more non-carbon atoms (e.g., O, N, S, Se, etc) in the backbone of a ring structure. Nitrogen-containing heteroaryl moieties include, but are not limited to, pyridine, imidazole, benzimidazole, pyrazole, pyrazine, triazine, pyrimidine, and the like.

“Aralkyl” refers to an -alkyl-aryl group, optionally wherein the alkyl and/or aryl moiety is substituted. An exemplary aralkyl group is benzyl, i.e., —CH₂C₆H₅.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

The term “arylene” refers to a bivalent aromatic group, e.g., a bivalent phenyl or napthyl group. The arylene group can optionally be substituted with one or more aryl group substituents and/or include one or more heteroatoms.

The term “amino” refers to the group —N(R)₂ wherein each R is independently H, alkyl, substituted alkyl, aryl, substituted aryl, aralkyl, or substituted aralkyl. The terms “aminoalkyl” and “alkylamino” can refer to the group —N(R)₂ wherein each R is H, alkyl or substituted alkyl, and wherein at least one R is alkyl or substituted alkyl. “Arylamine” and “aminoaryl” refer to the group —N(R)₂ wherein each R is H, aryl, or substituted aryl, and wherein at least one R is aryl or substituted aryl, e.g., aniline (i.e., —NHC₆H₅).

The term “thioalkyl” can refer to the group —SR, wherein R is selected from H, alkyl, substituted alkyl, aralkyl, substituted aralkyl, aryl, and substituted aryl. Similarly, the terms “thioaralkyl” and “thioaryl” refer to —SR groups wherein R is aralkyl and aryl, respectively.

The terms “halo”, “halide”, or “halogen” as used herein refer to fluoro, chloro, bromo, and iodo groups.

The terms “hydroxyl” and “hydroxy” refer to the —OH group.

The terms “mercapto” or “thiol” refer to the —SH group.

The terms “carboxylate” and “carboxylic acid” can refer to the groups —C(═O)O— and —C(═O)OH, respectively. The term “carboxyl” can also refer to the —C(═O)OH group. In some embodiments, “carboxylate” or “carboxyl” can refer to either the —C(═O)O⁻ or —C(═O)OH group.

The term “carbonate” refers to the —O—C(═O)—O— group.

The term “carbamate” refers to the —NR—C(═O)—O— group, wherein R can be H, alkyl, substituted alkyl, aralkyl, substituted aralkyl, aryl or substituted aryl.

The term “oxybenzyloxy” refers to the —O—CH₂—C₆H₄—O— group and to substituted derivatives there of wherein one of the hydrogen atoms is replaced by an alkyl or aryl group substituent.

The term “acetal” refers to a group comprising or consisting of —O—C(R)₂—O—, wherein each R group is independently H or an alkyl group substituent, e.g., alky or aryl. In some embodiments, an R group can be an alkylene or arylene group.

The term “phosphonate” as used herein refers to a compound or moiety of the structure R—P(═O)(OH)₂, wherein R can be independently alkyl, substituted alkyl, aralkyl, substituted aralkyl, aryl, or substituted aryl. Thus, phosphonate refers to a moiety wherein a carbon atom is attached to the phosphorus atom of a —P(═O)(OH)₂ group. In some embodiments, one or both of the hydrogen atoms are absent and replaced by a negative charge.

The term “phosphate” refers to a compound or moiety comprising the structure —O—P(═O)(OH)₂ or —NH—P(═O)(OH)₂, i.e., compounds or moieties wherein an oxygen atom or a nitrogen atom is attached to the phosphorous atom of a —P(═O)(OH)₂ group. In some embodiments, the phosphate comprises the structure RO—P(═O)(OH)₂ or RNH—P(═O)(OH)₂, where R is alkyl, substituted alkyl, aralkyl, substituted aralkyl, aryl, or substituted aryl. In some embodiments, one or both of the OH hydrogen atoms is absent and replaced by a negative charge.

The term “hydrophilic” can refer to a compound or chemical species or functional group that dissolves or preferentially dissolves in water and/or aqueous solutions.

The term “hydrophobic” refers to compounds, chemical species or functional groups, that do not significantly dissolve in water and/or aqueous solutions and/or which preferentially dissolve in fats and/or non-aqueous solutions.

Wavy lines, such as in the wavy line shown in the structure:

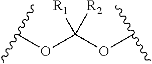

are used in the chemical formulas described herein to indicate the attachment site of the specified structure to another chemical group, for example, to a monovalent derivative of a drug compound or to the monovalent derivative of a lipid.

A dashed line representing a bond in a chemical formula indicates that the bond can be either present or absent. For example, the chemical structure:

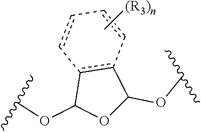

refers to groups where one or more bonds can be present or absent attached to and/or part of the five-membered ring. For example, a six-membered ring, optionally with one, two or three double bonds can be present or absent, and when present, is substituted by n substituents R₃. When none of the bonds represented by dashed lines are present, the group can have the structure:

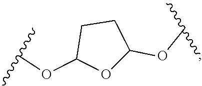

and
when all of the bonds represented by dashed lines are present, the group can have the structure:

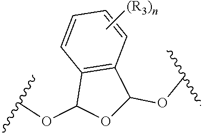

The term “monovalent” as used herein refers to a chemical moiety that has one site available for chemical bonding to another chemical moiety. Thus, a “monovalent moiety” can be a part of whole molecule that is attached to the remainder of the whole molecule via an attachment at one site on the monovalent moiety.

The term “bivalent” as used herein refers to a chemical moiety that has two sites available for chemical bonding to another chemical moiety or moieties.

The terms “conjugate” and “conjugated” as used herein can refer to the attachment (e.g., the covalent attachment) of two or more components (e.g., chemical compounds, polymers, biomolecule, particles, etc.) to one another. In some embodiments, a conjugate can comprise monovalent moieties derived from two different chemical compounds covalently linked via a bivalent linker moiety (e.g., an optionally substituted alkylene or arylene). In some embodiments, the linker can contain one or more biodegradable bond, such that one or more bonds in the linker can be broken when the prodrug is exposed to a particular physiological environment or enzyme.

The term “prodrug” as used herein, can refer to a compound that, upon administration to a subject or sample, is capable of providing (directly or indirectly) another compound (i.e., a “parent compound”) having a desired biological activity (e.g., anti-cancer activity). In some, but not all, embodiments, the prodrug compound has less of the desired biological activity than the parent compound. In some embodiments, the prodrug compound has no measurable biological activity prior to transformation to the parent compound. In some embodiments, the prodrug itself has the desired activity.

Transformation of the prodrug to the parent compound can take place in the presence of particular enzymes (e.g., esterases) and/or under certain biological conditions (e.g., at a physiologically relevant pH or in the presence of reducing agents present in a physiological environment). In some embodiments, the prodrug is initially transformed into another prodrug, which is then transformed (sometimes much more slowly) into the parent compound. Prodrugs can provide increased bioavailability and/or enhanced delivery to a biological compartment (e.g., a cancer cell, a lysosome, the brain or lymphatic system, etc.) relative to a parent compound. In some embodiments, the prodrug can enhance the solubility of the drug in a particular carrier of interest and/or be more compatible with a particular delivery platform or formulation than the parent compound.

The terms “bonding” or “bonded” and variations thereof can refer to either covalent, coordinative, or non-covalent bonding. In some cases, the term “bonding” refers to bonding via a coordinate bond. In some embodiments, the term “bonding” refers to a covalent bond. The term “conjugation” can refer to a bonding process, as well, such as the formation of a covalent linkage or a coordinate bond.

As used herein, the term “metal-organic framework” refers to a solid two- or three-dimensional network comprising both metal and organic components, wherein the organic components include at least one, and typically more than one carbon atom. In some embodiments, the material is crystalline. In some embodiments, the material is amorphous.

In some embodiments, the material is porous. In some embodiments, the metal-organic matrix material is a coordination polymer, which comprises repeating units of coordination complexes comprising a metal-based secondary building unit (SBU), such as a metal ion or metal complex, and a bridging polydentate (e.g., bidentate or tridentate) organic ligand. Thus, in some embodiments, the material contains more than one type of SBU or metal ion. In some embodiments, the material can contain more than one type of organic bridging ligand.

The term “nanoscale metal-organic framework” can refer to a nanoscale particle comprising an MOF.

A “coordination complex” is a compound in which there is a coordinate bond between a metal ion and an electron pair donor, ligand, or chelating group. Thus, ligands or chelating groups are generally electron pair donors, molecules or molecular ions having unshared electron pairs available for donation to a metal ion.

The term “coordinate bond” refers to an interaction between an electron pair donor and a coordination site on a metal ion resulting in an attractive force between the electron pair donor and the metal ion. The use of this term is not intended to be limiting, in so much as certain coordinate bonds also can be classified as having more or less covalent character (if not entirely covalent character) depending on the characteristics of the metal ion and the electron pair donor.

As used herein, the term “ligand” refers generally to a species, such as a molecule or ion, which interacts, e.g., binds, in some way with another species. More particularly, as used herein, a “ligand” can refer to a molecule or ion that binds a metal ion in solution to form a “coordination complex.” See Martell, A. E., and Hancock, R. D., Metal Complexes in Aqueous Solutions, Plenum: New York (1996), which is incorporated herein by reference in its entirety. The terms “ligand” and “chelating group” can be used interchangeably. The term “bridging ligand” can refer to a group that bonds to more than one metal ion or complex, thus providing a “bridge” between the metal ions or complexes. Organic bridging ligands can have two or more groups with unshared electron pairs separated by, for example, an alkylene or arylene group. Groups with unshared electron pairs, include, but are not limited to, —CO₂H, —NO₂, amino, hydroxyl, thio, thioalkyl, —B(OH)₂, —SO₃H, PO₃H, phosphonate, and heteroatoms (e.g., nitrogen, oxygen, or sulfur) in heterocycles. The term “ligand” can also refer biologically relevant molecules or macromolecules that preferentially bind to on another, e.g., antibodies and its target antigen; a biological receptor and a molecule that preferentially binds thereto, etc.

The term “coordination site” when used herein with regard to a ligand, e.g., a bridging ligand, refers to a unshared electron pair, a negative charge, or atoms or functional groups cable of forming an unshared electron pair or negative charge (e.g., via deprotonation under at a particular pH).

The terms “nanoscale particle,” nanomaterial,” and “nanoparticle” refer to a structure having at least one region with a dimension (e.g., length, width, diameter, etc.) of less than about 1,000 nm. In some embodiments, the dimension is smaller (e.g., less than about 500 nm, less than about 250 nm, less than about 200 nm, less than about 150 nm, less than about 125 nm, less than about 100 nm, less than about 80 nm, less than about 70 nm, less than about 60 nm, less than about 50 nm, less than about 40 nm, less than about 30 nm or even less than about 20 nm). In some embodiments, the dimension is between about 20 nm and about 250 nm (e.g., about 20, 30, 40, 50, 60, 70, 80, 90, 100, 110, 120, 130, 140, 150, 160, 170, 180, 190, 200, 210, 220, 230, 240, or 250 nm).

In some embodiments, the nanoparticle is approximately spherical. When the nanoparticle is approximately spherical, the characteristic dimension can correspond to the diameter of the sphere. In addition to spherical shapes, the nanomaterial can be disc-shaped, plate-shaped (e.g., hexagonally plate-like), oblong, polyhedral, rod-shaped, cubic, or irregularly-shaped.

The nanoparticle can comprise a core region (i.e., the space between the outer dimensions of the particle) and an outer surface (i.e., the surface that defines the outer dimensions of the particle). In some embodiments, the nanoparticle can have one or more coating layers surrounding or partially surrounding the nanoparticle core. Thus, for example, a spherical nanoparticle can have one or more concentric coating layers, each successive layer being dispersed over the outer surface of a smaller layer closer to the center of the particle. Such nanoparticles can be referred to as “core-shell” nanoparticles, wherein the shell refers to the coating layer or layers.

The term “nanoscale coordination polymer” or NCP can refer to a nanoscale particle comprising a coordination polymer, optionally a metal-phosphate coordination polymer, i.e., a polymer comprising repeating units of coordination complexes between a metal ion and mono- or bis-phosphate ligands.

In some embodiments, the presently disclosed nanoparticles can comprise a solid metal-organic framework (MOF) matrix, which are two- or three-dimensional networks of SBUs linked together by bridging ligands. The MOF can comprise one or more pores or hollow interior regions. The MOF matrix can be amorphous or crystalline. In some embodiments, the nanoparticle core further comprises one or more PSs, X-ray absorbing agents, scintillation agents and/or other therapeutic agents (e.g., anticancer or immunotherapy agents), which can be physically trapped within the matrix, coordinated to a metal ion of the matrix, or chemically bonded (e.g., to a organic bridging ligand in the matrix or a compound in a layer dispersed over the nanoparticle core) via a covalent or ionic bond. In some embodiments, a photosensitizer or a derivative thereof can be an organic bridging ligand or attached to an organic bridging ligand within a metal-organic matrix material that forms the core of the nanoparticle, while the metal of the SBU acts as a scintillator. Alternatively, the scintillator, X-ray absorbing agent and/or PS can be entrapped within the MOF or covalently attached to the MOF.

“Embedded” can refer to an agent that is bound, for example covalently bound or bound via a coordinative bond, inside the core of the particle (e.g., to a coordination site of a bridging ligand or to a metal ion of an SBU). Alternatively, agents can be “sequestered”, “entrapped”, or “trapped” (i.e., non-covalently encapsulated) inside pores, cavities or channels in the core of an MOF particle or interact with a MOF material via hydrogen bonding, London dispersion forces, or any other non-covalent interaction.

The term “small molecule” as used herein can refer to a non-polymeric, naturally-occurring or synthetic molecule. Small molecules typically have a molecular weight of about 900 Daltons (Da) or less (e.g., about 800 Da, about 750 Da, about 700 Da, about 650 Da, about 600 Da, about 550 Da, or about 500 Da or less).

The term “macromolecule” as used herein refers to molecules that are larger than about 900 Da. In some embodiments, the macromolecule is a polymer or biopolymer, e.g., a protein or a nucleic acid.

The terms “polymer” and “polymeric” refer to chemical structures that have repeating units (i.e., multiple copies of a given chemical substructure). Polymers can be formed from polymerizable monomers. A polymerizable monomer is a molecule that comprises one or more moieties that can react to form bonds (e.g., covalent or coordination bonds) with moieties on other molecules of polymerizable monomer. In some embodiments, each polymerizable monomer molecule can bond to two or more other molecules/moieties. In some cases, a polymerizable monomer will bond to only one other molecule, forming a terminus of the polymeric material.

Polymers can be organic, or inorganic, or a combination thereof. As used herein, the term “inorganic” refers to a compound or composition that contains at least some atoms other than carbon, hydrogen, nitrogen, oxygen, sulfur, phosphorous, or one of the halides. Thus, for example, an inorganic compound or composition can contain one or more silicon atoms and/or one or more metal atoms.

As used herein “organic polymers” are those that do not include silica or metal atoms in their repeating units. Exemplary organic polymers include polyvinylpyrrolidone (PVO), polyesters, polyamides, polyethers, polydienes, and the like. Some organic polymers contain biodegradable linkages, such as esters or amides, such that they can degrade overtime under biological conditions.

The term “hydrophilic polymer” as used herein generally refers to hydrophilic organic polymers, such as but not limited to, polyvinylpyrrolidone (PVP), polyvinylmethylether, polymethyloxazoline, polyethyloxazoline, polyhydroxy-propyloxazoline, polyhydroxypropylmethacrylamide, polymethyacrylamide, polydimethylacrylamide, polyhydroxylpropylmethacrylate, polyhydroxy-ethylacrylate, hydroxymethylcellulose, hydroxyethylcellulose, polyethylene-imine (PEI), polyethyleneglycol (i.e., PEG) or another hydrophilic poly(alkyleneoxide), polyglycerine, and polyaspartamide. As noted above, the term “hydrophilic” refers to the ability of a molecule or chemical species to interact with water. Thus, hydrophilic polymers are typically polar or have groups that can hydrogen bond to water.

The term “imaging agent” refers to a chemical moiety that aids in the visualization of a sample. For example, an imaging agent can be a “contrast agent”, and can refer to a moiety (a specific part of or an entire molecule, macromolecule, coordination complex, or nanoparticle) that increases the contrast of a biological tissue or structure being examined. The contrast agent can increase the contrast of a structure being examined using, for example, magnetic resonance imaging (MIRI), optical imaging, positron emission tomography (PET) imaging, single photon emission computed tomography (SPECT) imaging, or a combination thereof (i.e., the contrast agent can be multimodal).

The term “MRI contrast agent” refers to a moiety that effects a change in induced relaxation rates of water protons in a sample.

The terms “optical imaging agent” or “optical contrast agent” refer to a group that can be detected based upon an ability to absorb, reflect or emit light (e.g., ultraviolet, visible, or infrared light). Optical imaging agents can be detected based on a change in amount of absorbance, reflectance, or fluorescence, or a change in the number of absorbance peaks or their wavelength maxima. Thus, optical imaging agents include those which can be detected based on fluorescence or luminescence, including organic and inorganic dyes.

The terms “fluorophore” and “fluorescent moiety” refer to species that can be excited by visible light or non-visible light (e.g., UV light). Examples of fluorophores include, but are not limited to: quantum dots and doped quantum dots (e.g., a semiconducting CdSe quantum dot or a Mn-doped CdSe quantum dot), fluorescein, fluorescein derivatives and analogues, indocyanine green, rhodamine, triphenylmethines, polymethines, cyanines, phalocyanines, naphthocyanines, merocyanines, lanthanide complexes or cryptates, fullerenes, oxatellurazoles, LaJolla blue, porphyrins and porphyrin analogues and natural chromophores/fluorophores such as chlorophyll, carotenoids, flavonoids, bilins, phytochrome, phycobilins, phycoerythrin, phycocyanines, retinoic acid and analogues such as retinoins and retinates.

The term “photosensitizer” (PS) refers to a chemical compound or moiety that can be excited by light of a particular wavelength, typically visible or near-infrared (NIR) light, and produce a reactive oxygen species (ROS). For example, in its excited state, the photosensitizer can undergo intersystem crossing and transfer energy to oxygen (O₂) (e.g., in tissues being treated by PDT) to produce ROSs, such as singlet oxygen (¹O₂). Any known type of a photosensitizer can be used in accordance with the presently disclosed subject matter. In some embodiments, the photosensitizer is a porphyrin, a chlorophyll, a dye, or a derivative or analog thereof. In some embodiments, phophyrins, chlorins, bacteriochlorins, or porphycenes can be used. In some embodiments, the photosensitizer can have one or more functional groups, such as carboxylic acid, amine, or isothiocyanate, e.g., for using in attaching the photosensitizer to another molecule or moiety, such as an organic bridging ligand or a SBU, and.or for providing an additional site or sites to enhance coordination or to coordinate an additional metal or metals. In some embodiments, the photosensitizer is a porphyrin or a derivative or analog thereof. Exemplary porphyrins include, but are not limited to, hematoporphyrin, protoporphyrin and tetraphenylporphyrin (TPP). Exemplary porphyrin derivatives include, but are not limited to, pyropheophorbides, bacteriochlorophylls, chlorophyll a, benzoporphyrin derivatives, tetrahydroxyphenyl chlorins, purpurins, benzochlorins, naphthochlorins, verdins, rhodins, oxochlorins, azachlorins, bacteriochlorins, tolyporphyrins and benzobacteriochlorins. Porphyrin analogs include, but are not limited to, expanded porphyrin family members (such as texaphyrins, sapphyrins and hexaphyrins), porphyrin isomers (such as porphycenes, inverted porphyrins, phthalocyanines, and naphthalocyanines), and TPP substituted with one or more functional groups.

The term “pyrolipid” refers to a conjugate of a lipid and a porphyrin, porphyrin derivative, or porphyrin analog. In some embodiments, the pyrolipid can comprise a lipid conjugate wherein a porphyrin or a derivative or analog thereof is covalently attached to a lipid side chain. Pyrolipids and pyrolipid synthesis are described, for example, in U.S. Patent Application Publication No. 2014/0127763, which is incorporated herein by reference in its entirety.

The term “lyso-lipid” refers to a lipid in which one or more acyl group has been removed.

The term “cancer” as used herein refers to diseases caused by uncontrolled cell division and/or the ability of cells to metastasize, or to establish new growth in additional sites. The terms “malignant”, “malignancy”, “neoplasm”, “tumor,” “cancer” and variations thereof refer to cancerous cells or groups of cancerous cells.

Particular types of cancer include, but are not limited to, skin cancers (e.g., melanoma), connective tissue cancers (e.g., sarcomas), adipose cancers, breast cancers, head and neck cancers, lung cancers (e.g., mesothelioma), stomach cancers, pancreatic cancers, ovarian cancers, cervical cancers, uterine cancers, anogenital cancers (e.g., testicular cancer), kidney cancers, bladder cancers, colorectal cancers (e.g., colon cancers, colorectal adenocarcinomas, etc.), prostate cancers, central nervous system (CNS) cancers, retinal cancer, blood, neuroblastomas, multiple myeloma, and lymphoid cancers (e.g., Hodgkin's and non-Hodgkin's lymphomas).

The term “metastatic cancer” refers to cancer that has spread from its initial site (i.e., the primary site) in a patient's body.

The terms “anti-cancer drug”, “chemotherapeutic”, and “anti-cancer prodrug” refer to drugs (i.e., chemical compounds) or prodrugs known to, or suspected of being able to treat a cancer (i.e., to kill cancer cells, prohibit proliferation of cancer cells, or treat a symptom related to cancer). In some embodiments, the term “chemotherapeutic” as used herein refers to a synthetic or naturally occurring small molecule (e.g., less than 1500 Dalton (Da), less than 1250 Da, less than 1000 Da, less than 900 Da, less than 800 Da, or less than 750 Da, less than 700 Da, less than 650 Da, less than 600 Da, etc.) or a derivative thereof that is used to treat cancer and/or that has cytotoxic ability. In some embodiments, the term “chemotherapeutic” refers to a platinum coordination complex. Such more traditional or conventional chemotherapeutic agents can be described by mechanism of action or by chemical compound class, and can include, but are not limited to, alkylating agents (e.g., melphalan), anthracyclines (e.g., doxorubicin), cytoskeletal disruptors (e.g., paclitaxel), epothilones, histone deacetylase inhibitors (e.g., vorinostat), inhibitors of topoisomerase I or II (e.g., irinotecan or etoposide), kinase inhibitors (e.g., bortezomib), nucleotide analogs or precursors thereof (e.g., methotrexate), peptide antibiotics (e.g., bleomycin), platinum-based agents (e.g., platinum coordination complexes, such as cisplatin, oxaliplatin, or carboplatin), retinoids (e.g., tretinoin), and vinka alkaloids (e.g., vinblastine). In some embodiments, the term “chemotherapeutic” refers to a hormone or polypeptide chemotherapeutic drug.

The term “scintillator” refers to a moiety or compound that exhibits luminescence (emits light, e.g., light in the visible or NIR range) when excited by ionizing radiation, such as x-rays.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the presently disclosed subject matter refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

### II. General Considerations

The presently disclosed subject matter relates, in some aspects, to the design of lipid-based prodrugs and to nanoparticles (e.g., core-shell nanoscale coordination polymers (NCPs) or other nanoscale metal-organic frameworks (MOFs)) for delivery of the prodrugs. In some embodiments, the nanoparticles can be used for the co-delivery of drug combinations, such as hydrophilic and hydrophobic drug combinations. Hydrophilic drugs, such as cisplatin, carboplatin, oxaliplatin, and gemcitabine, and/or their prodrugs can be incorporated into the core of NCP particles. For example, bisphosphates of the metal coordination complexes cisplatin, carboplatin and oxaliplatin can be prepared and copolymerized with a metal ion to provide a NCP comprising a metal bisphosphate coordination polymer that can form a nanoparticle core or the hydrophilic drugs can be included in solutions used to prepare NCP particles (e.g., via copolymerization of metal ions and non-therapeutic phosphonates), thereby resulting in the hydrophilic drugs being embedded within the NCP core (e.g., physically entrapped within pores in the NCP core).

The lipid-based prodrugs of the presently disclosed subject matter can be prodrugs of hydrophobic drugs (e.g., hydrophobic chemotherapeutic drugs). These prodrugs can be synthesized to include cleavable carbonate or carbamate linkages. For example, the cleavable linkages can comprise an acetal group where one of the oxygen atoms of the acetal group is also part of a carbonate or carbamate bond (i.e., is directly attached to the carbon atom of the carbonate or carbamate group). The prodrugs can also be prepared using oxybenzyloxy groups where the oxygen directly attached to the benzyl carbon atom is also directly attached to the carbon atom of a carbonate or carbamate group. These prodrugs can be sensitive to the acidic environment of cancer cells, as well as to esterases, enhancing release of the drug moiety in a cancer cell.

According to some aspects of the presently disclosed subject matter, prodrugs of hydrophilic and hydrophobic drugs self-assemble into core-shell NCPs to allow for slow and triggered release of each drug. These nanoparticles can improve the pharmacodynamic profile of each drug, increasing drug exposure to cancer cells, while preventing premature degradation. The limited free drug exposure and metabolism in the blood, spleen, and liver with simultaneous increase and sustainment of active drug accumulation in the tumor can afford superior anti-cancer efficacy. In some embodiments, the lipid-based prodrugs can target the low-density lipoprotein receptor (LDL) and/or the nanoparticles can adsorb apoliprotein B-100 (ApoB-100), resulting in enhanced delivery of the nanoparticle and any prodrugs associated therewith to a cancer cell, resulting in increased cancer cell drug exposure.

Some small molecule chemotherapeutics, including oxaliplatin (OxPt), paclitaxel (PTX), daunorubicin, docetaxel, doxorubicin, cyclophosphamide, dihydroartemisinin, and mitoxanthrone, can efficiently cause immunogenic cell death. These chemotherapeutic agents can be immune-stimulatory. In some embodiments, combination chemotherapy regimens of these NCP particles can synergize with immunotherapies, such as immune checkpoint inhibitors. For instance, combination of the presently disclosed core-shell NCPs with immune checkpoint inhibitors can activate tumor microenvironments to elicit systemic antitumor immune response, further enhancing anticancer efficacy.

Accordingly, in some embodiments, the presently disclosed subject matter provides a prodrug comprising a structure of the formula D-BL-L, wherein B is a monovalent drug moiety, BL is a bivalent linker, and L is a monovalent lipid moiety. D can be the monovalent derivative of any drug of interest that comprises a hydroxyl or amino group. The oxygen atom of the deprotonated hydroxyl group or the nitrogen atom of the deprotonated amino group can serve as an atom of a carbonate (i.e., —O—C(═O)—O—) or carbamate (i.e., —NR—C(═O)—O—, where R is H or an alkyl, aralkyl or aryl group) bond. In some embodiments, BL is free of a disulfide bond. In some embodiments, the prodrug is free of a disulfide bond.

In some embodiments, D is a monovalent derivative of an anti-cancer drug (e.g., an anti-cancer small molecule or polypeptide). In some embodiments, D is a monovalent moiety of a hydrophobic drug (e.g., a hydrophobic anti-cancer drug) In some embodiments, D is a monovalent derivative of a drug compound selected from the group including, but not limited to, Etoposide (ET), Podophyllotoxin (PPX), Paclitaxel (PTX), Docetaxel (DTX), dihydroartemisin (DHA), Camptothecin (CPT), 7-ethyl-10-hydroxycamptothecin (SN38), Topotecan, Doxorubicin, Epirubicin, Idarubicin, Vincristine, Mitoxantrone, Artesunate, Capecitabine, Octreotide, Leuprolide, and Goserelin.

BL is a bivalent linker wherein D is directly attached to BL via a carbonate or carbamate group, and wherein BL comprises at least one of an acetal group and a substituted oxybenzyloxy group, wherein an oxygen atom of the acetal group or the benzyl oxygen atom of the oxybenzyloxy group is directly attached to the carbon atom of a carbonate or carbamate group (optionally the carbonate or carbamate group directly attached to the drug moiety). In some embodiments, the acetal group has a structure of one of the formulas:

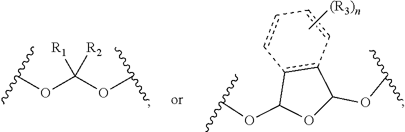

wherein: bonds represented by dashed lines can be present or absent, n is an integer between 0 and 4; R₁ and R₂ are independently selected from the group comprising H, alkyl, substituted alkyl, aralkyl, substituted aralkyl, aryl, and substituted aryl, each R₃ is an aryl group substituent, e.g., independently selected from alkyl, substituted alkyl, aralkyl, substituted aralkyl, aryl, substituted aryl, halo (e.g., Cl, Br, I, or F), alkoxy, aryloxy, hydroxy, acyl, carboxylate, phosphate, nitro, —N₃, B(OH)₂, and cyano; and wherein at least one of the oxygen atoms in the acetal group is directly bonded to a carbon atom of a carbonate or carbamate group.

In some embodiments, BL further comprises one or more additional alkylene or arylene moieties (e.g., directly attached to the oxygen atom of the acetal group that is not directly attached to the carbonate or carbamate group or directly attached the oxygen atom of the oxybenzyloxy group that is attached to the phenyl ring). For example, the additionally alkylene group can have the structure —C(═O)—(CH)ₘ—C(═O)—O— wherein m is an integer between 1 and 12 (e.g., 1, 2, 3, 4, 5, 6, 7, 8, 9, 10, 11, or 12). In some embodiments, BL comprises both an acetal group and an oxybenzyloxy group. In some embodiments, the prodrug can comprise more than one monovalent drug moiety D, wherein each drug moiety D is attached to BL via a carbamate or carbonate bond. For example, in some embodiments, the prodrug can comprise two or three drug moieties D, which can be the same or different.

In some embodiments, the acetal group has the structure:

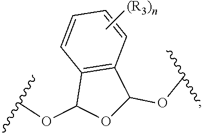

wherein n is an integer between 0 and 4 (i.e., 0, 1, 2, 3, or 4); R₁ and R₂ are independently selected from the group comprising H, alkyl, substituted alkyl, aralkyl, substituted aralkyl, aryl, and substituted aryl, each R₃ is an aryl group substituent, e.g., independently selected from the group comprising alkyl, substituted alkyl, aralkyl, substituted aralkyl, aryl, substituted aryl, halo (e.g., Cl, Br, I, or F), alkoxy, aryloxy, hydroxy, acyl, carboxylate, phosphate, nitro, —N₃, B(OH)₂, or cyano; and wherein at least one of the oxygen atoms in the acetal group is directly bonded to a carbon atom of a carbonate or carbamate group. In some embodiments, n is 0 and the acetal group has a structure of the formula:

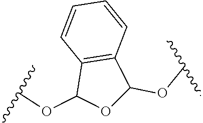

In some embodiments, the substituted oxybenzyloxy group has a structure of the formula:

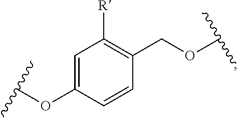

wherein R′ is an aryl group substituent, and wherein the oxygen atom attached to the benzyl carbon of the oxybenzyloxy group is directly bonded to a carbon atom of a carbonate or carbamate group. In some embodiments, R′ is selected from the group comprising nitro, —N₃, and —B(OH)₂.

In some embodiments, L is a monovalent derivative of cholesterol, oleic acid, a lyso-lipid, or phosphocholine.

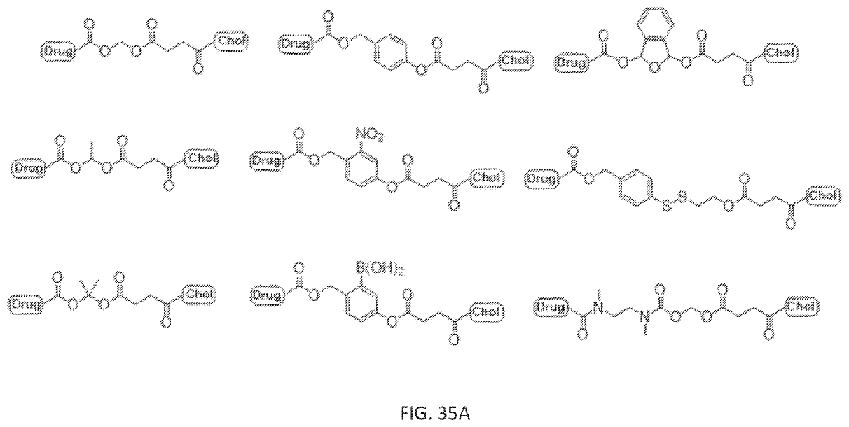

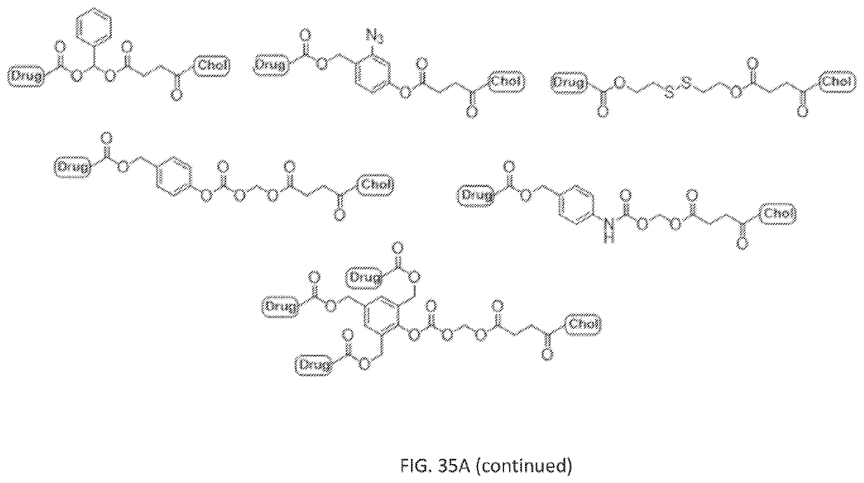

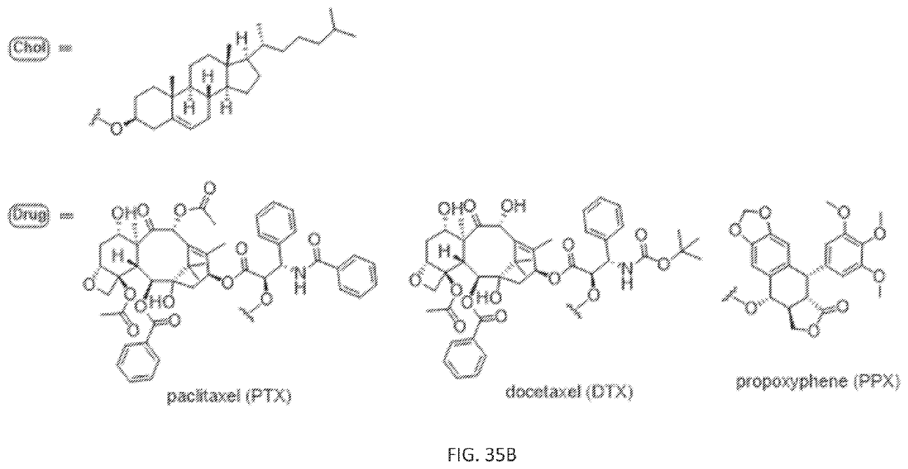

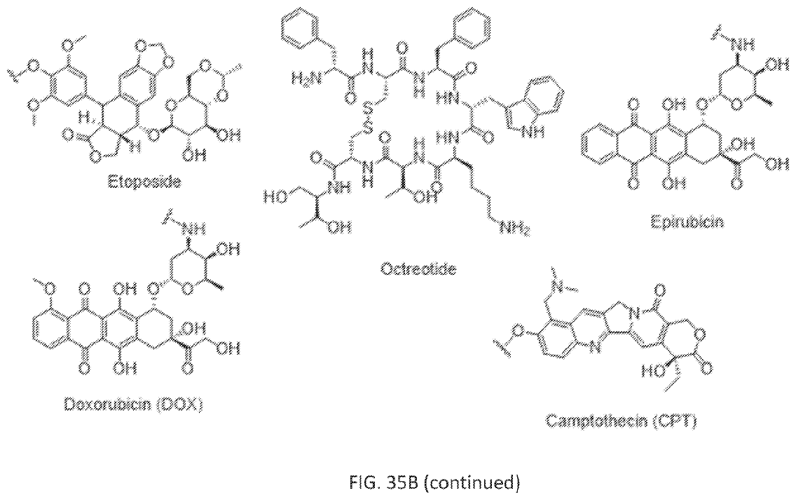

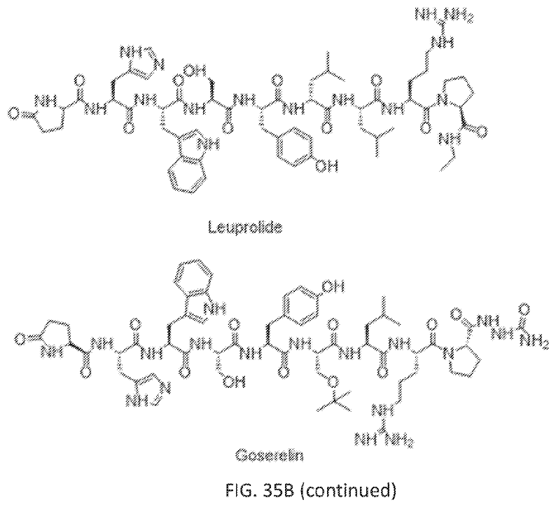

In some embodiments, the prodrug comprises a structure shown in FIG. 35A. In some embodiments, the prodrug comprises a structure of one of the formulas:

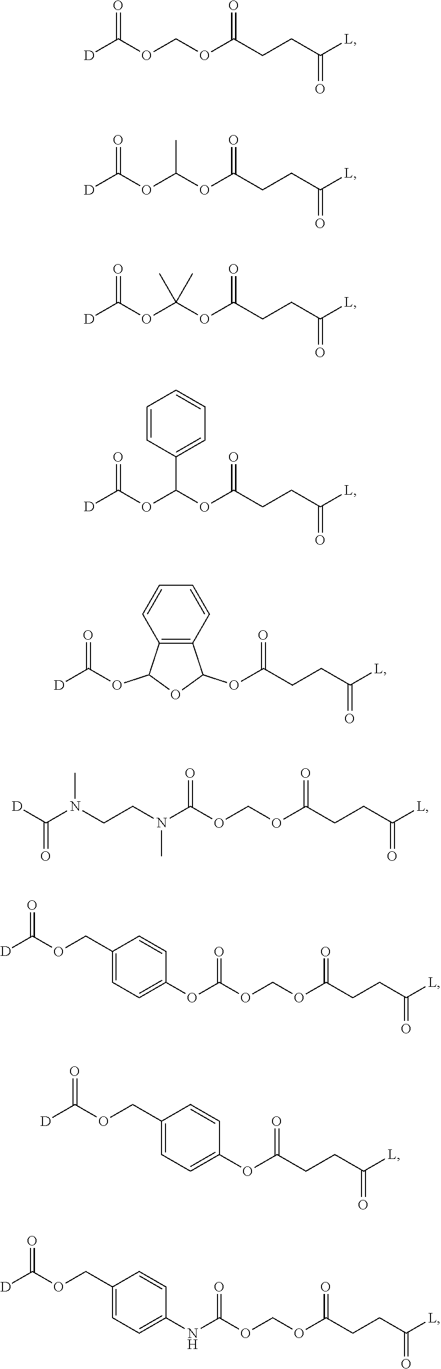

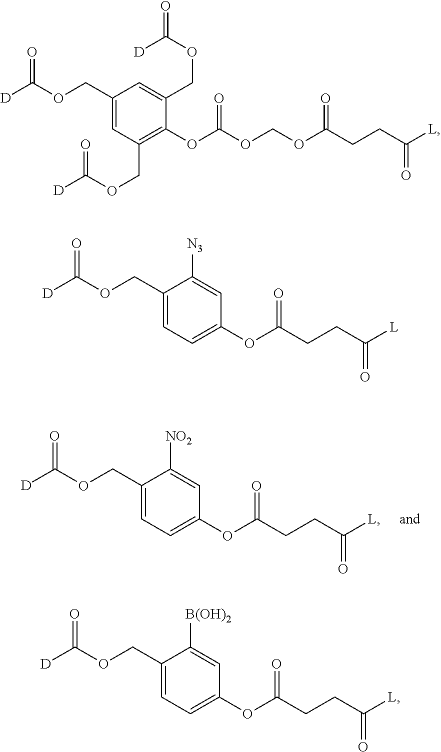

wherein D is a monovalent drug moiety (e.g., a monovalent chemotherapeutic drug moiety, such as a monovalent chemotherapeutic drug moiety as shown in FIG. 35B) and L is a monovalent lipid moiety (e.g., the monovalent cholesterol moiety shown in FIG. 35B).

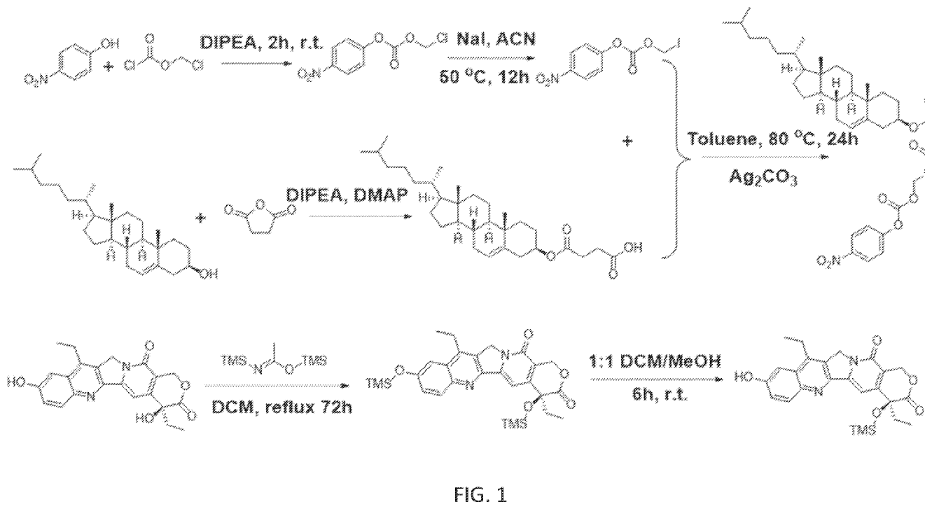

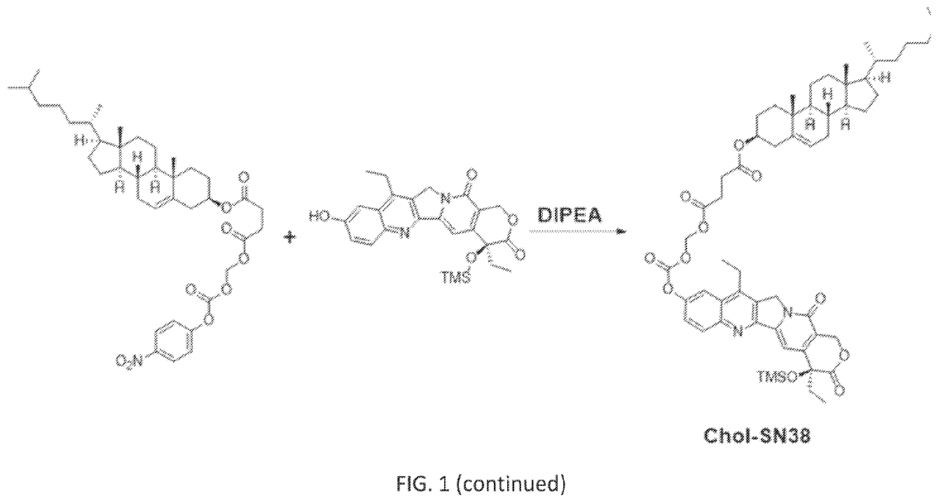

FIG. 1 shows an exemplary synthetic route to a prodrug of the presently disclosed subject matter comprising an acetal group directly attached to a carbonate bond. While FIG. 1 more particularly shows the synthesis of a cholesterol-based prodrug of SN38, other lipids and/or drugs can be used in place of the cholesterol and/or SN38. See also Synthetic Pathway 1 in Scheme 1, below. Generally, a lipid with a hydroxyl group can be reacted with succinic anhydride in the presence of a sterically hindered base, such as a trialkylamine (e.g., di-isopropylethylamine (DIPEA)), and a nucleophilic catalyst, such as dimethylaminopyridine (DMAP), in a nonprotic solvent to provide a lipid moiety with an ester linkage to an alkylene group, where the alkylene group ends in a terminal carboxylic acid moiety. The succinic anhydride can be replaced with another anhydride or with a dicarboxylic acid, if a different alkylene length is desired. Alternatively, succinic anhydride can be replaced by a carboxylic acid with a suitable chemical functional group that can be transformed into a carboxylic acid moiety in a further step. For example, a protected terminal hydroxyl group of a carboxylic acid with an alkylene moiety linking the carboxylic acid and the protected terminal hydroxyl group can be deprotected and reacted with an oxidant to provide a carboxylic acid.

In any case, the terminal carboxylic acid group of the moiety newly added to the lipid can then be reacted with a halomethyl 4-nitrophenyl carbonate (e.g., an iodomethyl 4-nitrophenyl carbonate) or an analog thereof comprising a substituent (e.g., methyl or phenyl) or substituents attached to the methylene carbon atom and/or a different leaving group in place of the 4-nitrophenyl group. For instance, to prepare an acetal with a methyl group attached to the methylene carbon atom, 1-chloroethyl chloroformate can be reacted with 4-nitrophenyl carbonate in the presence of a sterically hindered base and then transformed, if desired, into the corresponding iodo compound (i.e., 1-iodoethyl 4-nitrophenyl carbonate) using NaI, e.g., using a phase transfer catalyst such as tetrabutylammonium bromide (TBABr) to provide a reagent with a better halide leaving group. The 4-nitrophenyl carbonate product of the reaction of the lipid with the terminal carboxylic acid group and the halomethyl 4-nitrophenyl carbonate can then be reacted with a drug comprising a hydroxyl or amino group in the presence of a sterically hindered base (e.g., DIPEA) to provide a new carbonate or carbamate bond, with the drug moiety replacing the 4-nitrophenyl group.

Lipids comprising carboxylic acid groups can be reacted directly with a halomethyl 4-nitrophenyl carbonate (e.g., an iodomethyl 4-nitrophenyl carbonate) or analog thereof to provide a lipid-linker 4-nitrophenyl carbonate-containing intermediate with an acetal group directly attached to a carbonate. This intermediate can then be reacted with a drug comprising an amino or hydroxyl group to provide a new carbonate or carbamate bond, with the drug moiety replacing the 4-nitrophenyl group

Prodrugs comprising oxybenzyloxy groups (either alone or in combination with acetal groups) can be prepared using routes described in Example 8, below. See also Synthetic Pathway 2 in Scheme 1, below. While 4-hydroxybenzyl alcohol is used to react with acyl chlorides prepared from lipid moieties or with 4-nitrophenyl carbonates of lipid-acetal-containing linker intermediates in Example 8, substituted analogs of 4-hydroxybenzyl alcohol can also be used to provide linkers with aryl-substituted oxybenzyloxy groups.

Prodrugs with acetal groups comprising fused ring structures can be prepared as shown in Synthetic Pathway 3 of Scheme 1, below. For example, a suitable benzene dicarboxylic acid can be reacted with a lipid moiety comprising a carboxylic acid in the presence of trifluoroacetic acid (TFA) to provide an intermediate alcohol further comprising a fused ring system. Then, the intermediate alcohol can be transformed into a 4-nitrophenyl carbonate using 4-nitrophenyl chloroformate to provide a 4-nitrophenyl carbonate. The 4-nitrophenyl carbonate can then be reacted with a hydroxyl group of a drug to provide a carbonate-containing prodrug. Alternatively, the 4-nitrophenyl carbonate can be reacted with an amino group of a drug to provide a carbamate-containing prodrug.

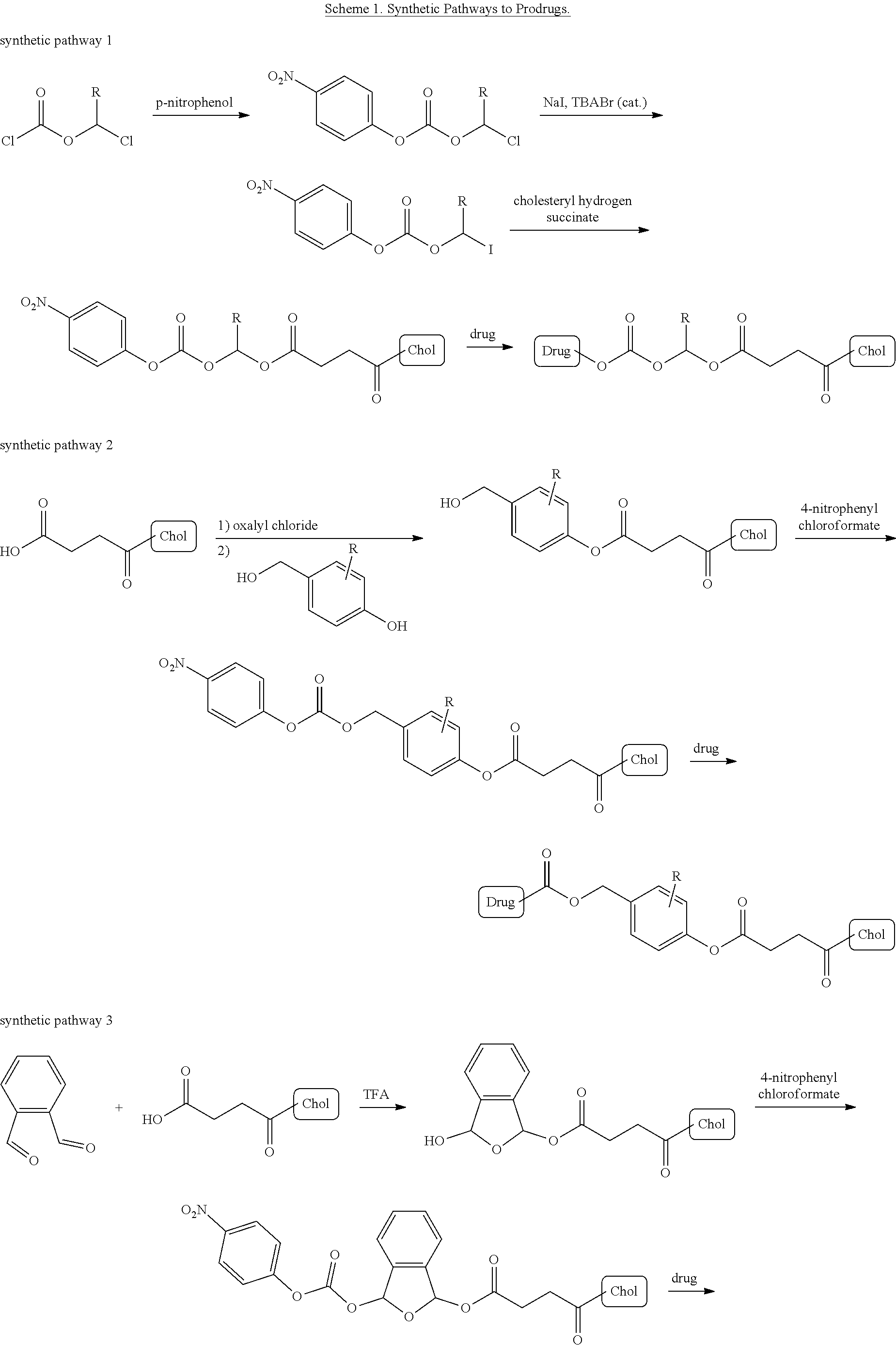

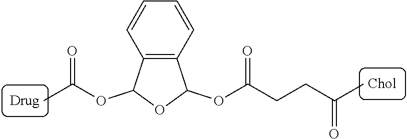 [SMILES: CC(=O)CCC(=O)OC1OC(OC(C)=O)c2ccccc21]

In some embodiments, BL comprises an acetal group having a structure of the formula:

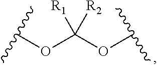

wherein R₁ and R₂ are independently selected from the group comprising H, alkyl (e.g., C₁-C₆ alkyl), substituted alkyl, aralkyl (e.g., benzyl), substituted aralkyl (e.g., substituted benzyl), aryl (e.g., phenyl), and substituted aryl (e.g., substituted phenyl). In some embodiments, R₁ and R₂ are the same. In some embodiments, R₁ and R₂ are different. In some embodiments, R₁ and R₂ are independently selected from the group comprising H, methyl, and phenyl. In some embodiments, at least one of R₁ and R₂ is H. In some embodiments, both R₁ and R₂ are H.

In some embodiments, L is an oleic acid moiety and L and BL together have the structure:

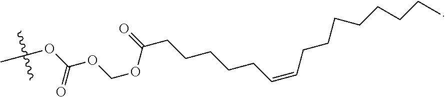 [SMILES: *OC(=O)OCOC(=O)CCCCC/C=C\CCCCCCCC]

In some embodiments, L is a cholesterol derivative and L and BL together have the structure:

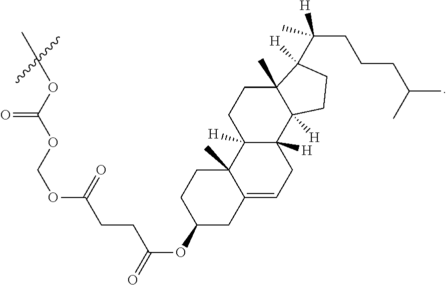 [SMILES: *OC(=O)OCOC(=O)CCC(=O)O[C@H]1CC[C@@]2(C)C(=CC[C@H]3[C@@H]4CC[C@H]([C@H](C)CCCC(C)C)[C@@]4(C)CC[C@@H]32)C1]

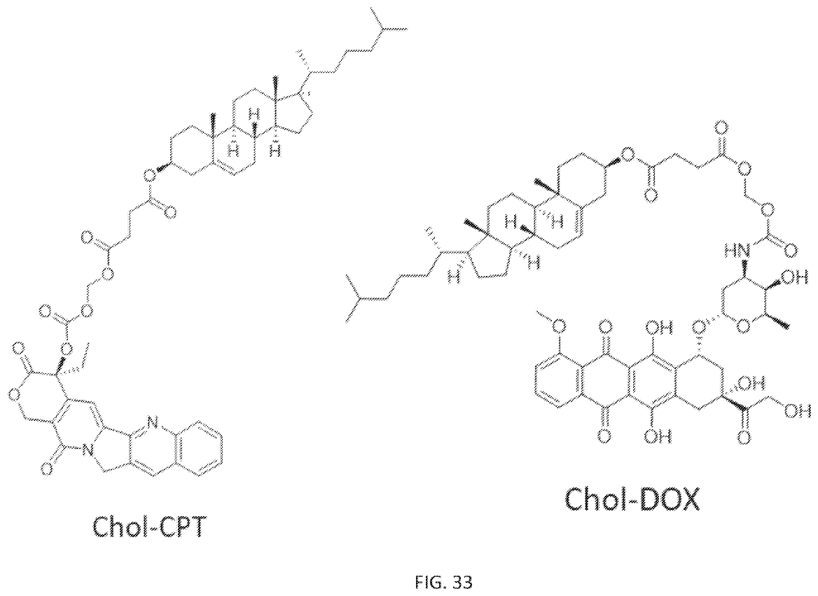

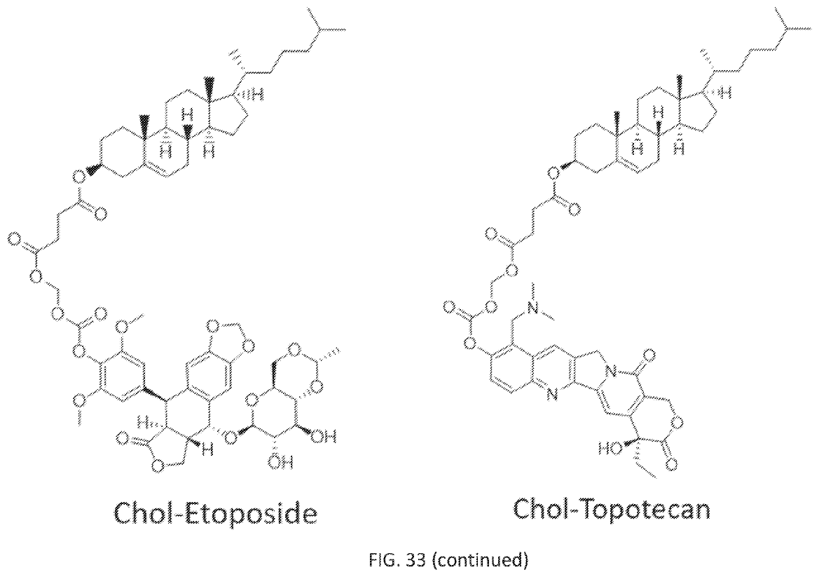

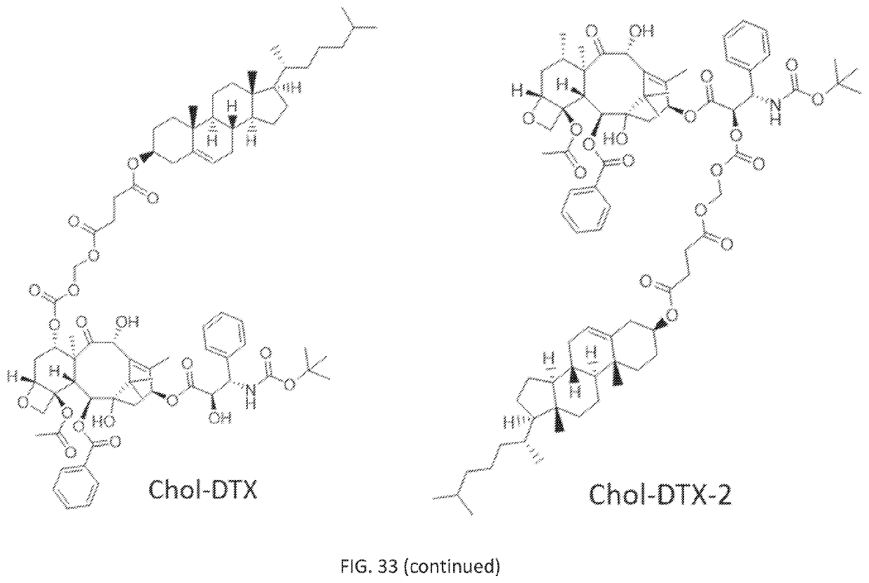

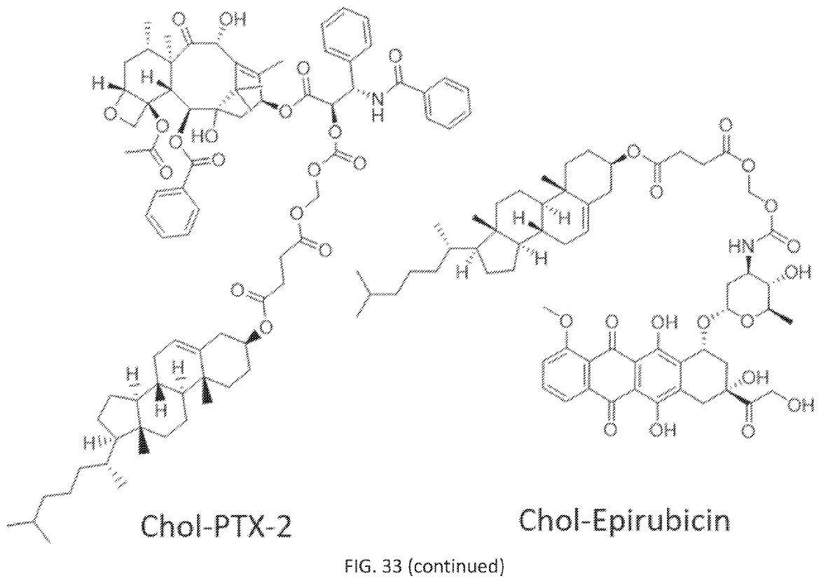

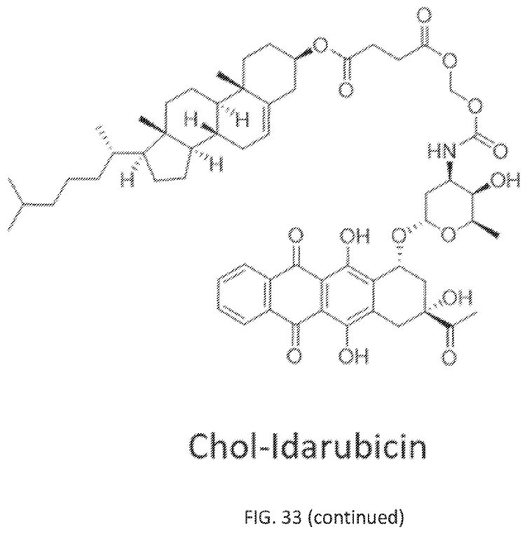

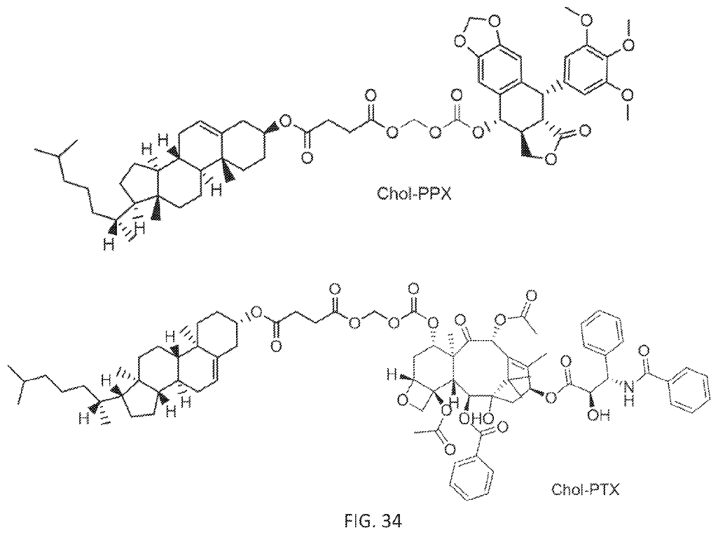

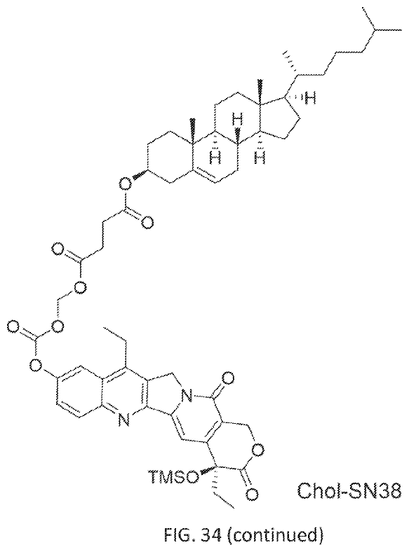

In some embodiments, the prodrug is a prodrug selected from one of the prodrugs shown in FIG. 33 or FIG. 34. In some embodiments, the prodrug is selected from the group comprising:

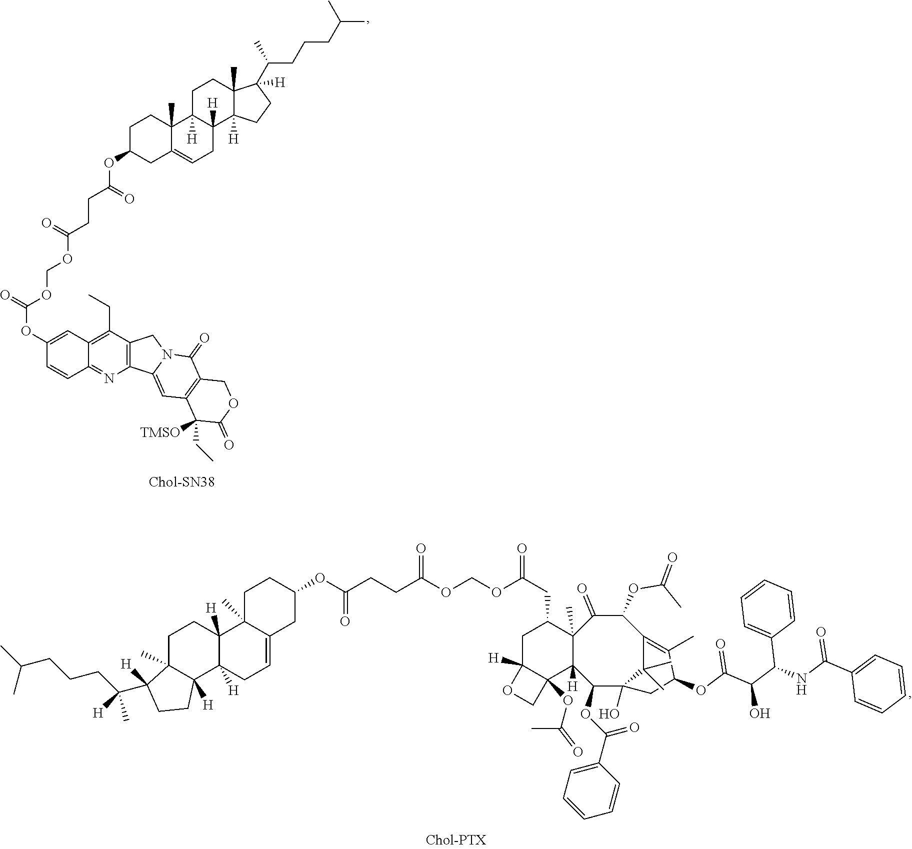

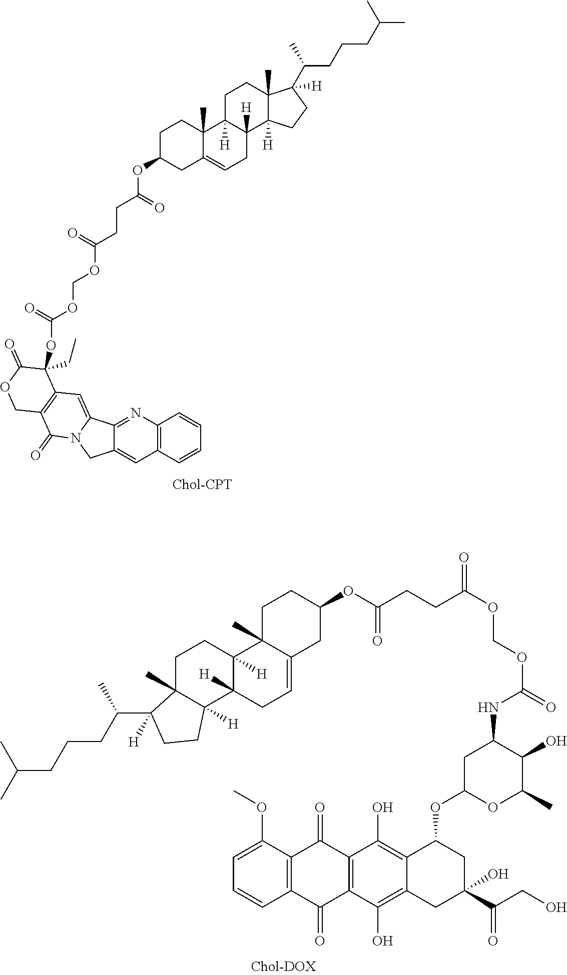

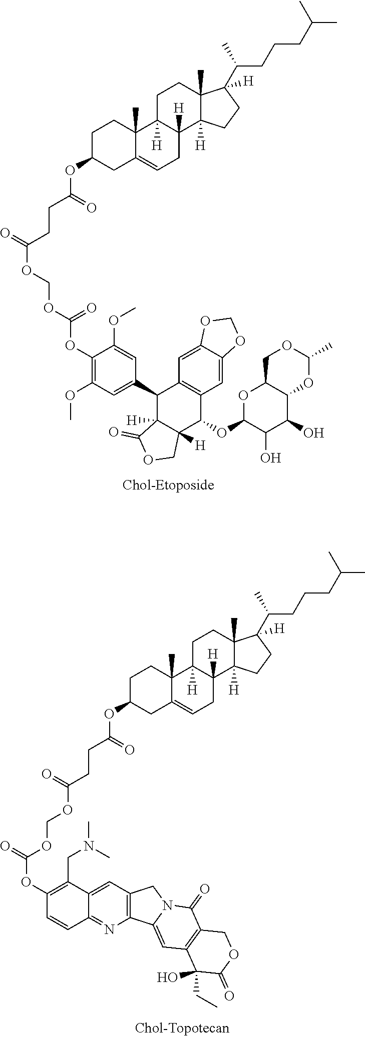

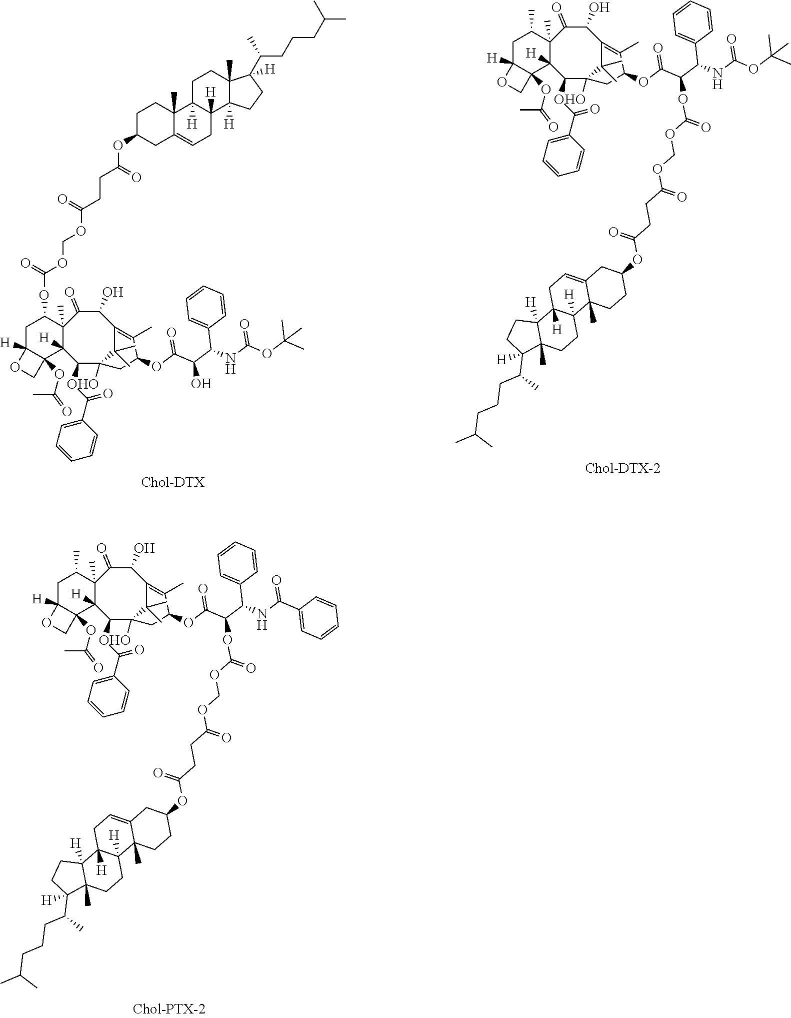

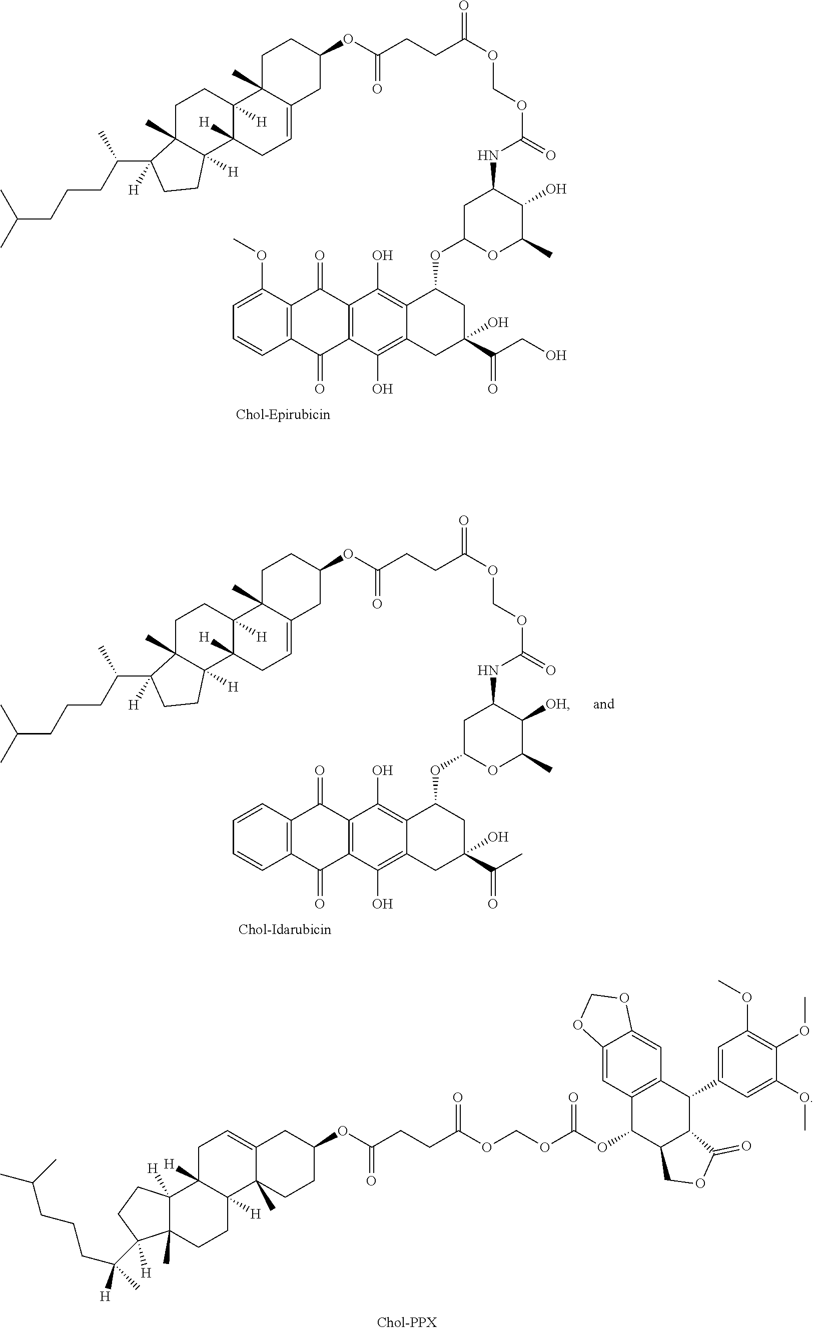

In some embodiments, the prodrug binds to low-density lipoprotein (LDL) (e.g., in plasma) and is actively transported to tumors via LDL receptor (LDLR)-mediated endocytosis. In some embodiments, the prodrug preferentially binds to LDL compared to albumin. In some embodiments, the prodrug has an association constant Kₐ for LDL that is at least about 5, 10, 25, 50, 100, 250, 500, or 750 times or more the Kₐ of the prodrug for albumin. In some embodiments, the prodrug has a Kₐ for LDL that is at least about 1000 times the Kₐ of the prodrug for albumin. In some embodiments, the prodrug has a Kₐ for LDL that is at least about 2000 times that of the Kₐ of the prodrug for albumin.

In some embodiments, the presently disclosed subject matter provides a nanoparticle comprising: (a) a core comprising a metal-organic matrix material, and (b) a coating layer covering at least a portion of the surface of the core, wherein the coating layer comprises a lipid layer or a lipid bilayer, wherein said lipid layer or lipid bilayer further comprises a prodrug of the formula D-BL-L as disclosed herein. In some embodiments, the metal-organic matrix material comprises a nanoscale coordination polymer (NCP). In some embodiments, the NCP comprises a metal-bisphosphate or metal-bisphosphonate coordination polymer comprising a multivalent metal ion and a bisphosphate or bisphosphonate. In some embodiments, the NCP comprises a metal-bisphosphate coordination polymer comprising a multivalent metal ion and a bisphosphate. In some embodiments, the NCP core comprises between about 40 and about 50 weight % of bisphosphate (e.g., about 40, 42, 44, 46, 48, or about 50 weight % bisphosphate). In some embodiments, the multivalent metal ion is an ion selected from the group comprising Ca²⁺, Mg²⁺, Mn²⁺, Zn²⁺ and combinations thereof. In some embodiments, the multivalent metal ion is Zn²⁺.

In some embodiments, the bisphosphate or bisphosphonate comprises or consists of a drug or a prodrug. In some embodiments, the bisphosphate or bisphosphonate is a prodrug of an anti-cancer agent. In some embodiments, the bisphosphate or bisphosphonate is a prodrug of cisplatin, oxaliplatin, or carboplatin. In some embodiments, the bisphosphate or bisphosphonate is a bisphosphate or bisphosphonate ester or other prodrug of cis, cis-trans-[Pt(NH₃)₂Cl₂(OH)₂] (i.e., a cisplatin prodrug), cis, trans-[Pt(dach)(oxalate)(OH)₂] (i.e., an oxaliplatin prodrug) or [Pt(cyclobutene dicarboxylic acid)(NH₃)₂(OH)₂] (i.e., a carboplatin prodrug). In some embodiments, the bisphosphate comprises ligands derived from phosphoramidic acid, e.g., a bisphosphate wherein two OH ligands of a platinum coordination complex such as cis, cis-trans-[Pt(NH₃)₂Cl₂(OH)₂], cis, trans-[Pt(dach)(oxalate)(OH)₂], or [Pt(cyclobutane dicarboxylic acid)(NH₃)₂(OH)₂] are replaced by ligands comprising the structure ⁻O—C(═O)—NH—P(═O)(OH)₂. Thus, in some embodiments, the nanoparticle comprises a core comprising an anti-cancer agent prodrug attached to the core via coordinative bonds. As used herein, the phrases “core comprising OxPt” or “core comprising carboplatin” can refer to a core that can deliver OxPt or carboplatin, i.e., nanoparticle cores comprising a OxPt prodrug or a carboplatin prodrug and/or nanoparticle cores comprising OxPt or carboplatin.

In some embodiments, the nanoparticle core comprises an embedded anti-cancer agent (e.g., physically or chemically sequestered in pores in the nanoparticle core). In some embodiments, the embedded anti-cancer agent is an embedded hydrophilic anti-cancer agent. In some the embedded anti-cancer agent is gemcitabine (GEM) or gemcitabine monophosphate (GMP). In some embodiments, an embedded anti-cancer agent is cytarabine monophosphate or arsenic acid.

In some embodiments, the nanoparticle core comprises at least two anti-cancer agents (e.g., two, three, four, five or more anti-cancer agents). In some embodiments, the at least two anti-cancer agents comprise a first anti-cancer agent, wherein the first anti-cancer agent is a cisplatin, carboplatin or oxaliplatin prodrug, and a second anti-cancer agent, wherein the second anti-cancer agent is an embedded, hydrophilic anti-cancer agent. In some embodiments, the first anti-cancer agent is a cisplatin, carboplatin or oxaliplatin bisphosphate prodrug. In some embodiments, the first anti-cancer agent is copolymerized with a multivalent metal ion to form a NCP that forms all or part of the core of the nanoparticle.

In some embodiments, the nanoparticle core comprises a metal bisphosphate coordination polymer comprising a multivalent metal ion and a bisphosphate, wherein said bisphosphate is an oxaliplatin prodrug having the structure Pt(dach)(oxalate)(bisphosphoramidic acid); and wherein the coating layer is a lipid bilayer comprising a prodrug of SN38 (e.g., a cholesterol prodrug of SN38). In some embodiments, the prodrug of SN38 has the structure:

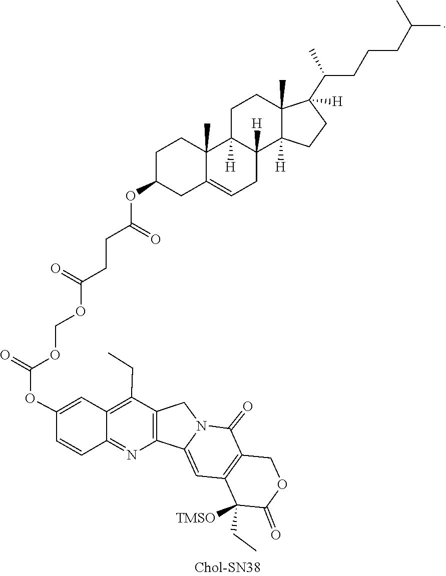

In some embodiments, the multivalent metal ion is Zn²⁺. In some embodiments, the nanoparticle core further a second anti-cancer agent (e.g., in addition to the oxaliplatin prodrug). In some embodiments, the nanoparticle core comprises GMP embedded in the nanoparticle core.

In some embodiments, the coating layer further comprises one or more of cholesterol, 1,2-dioleoyl-sn-glycero-3-phosphate sodium salt (DOPA), 1,2-distearoyl-sn-glycero-3-phosphoethanolamine (DSPE), 1,2-dioleoyl-3-trimethylammonium propane (DOTAP), and 1,2-dioleoyl-sn-glycero-3-phosphocholine (DOPC) and/or pegylated derivatives thereof (e.g., DSPE-PEG₂₀₀₀). In some embodiments, the coating layer comprises (in addition to the lipid-based prodrug), a mixture of cholesterol, DOPC, and DSPE-PEG₂₀₀₀.

In some embodiments, the coating layer comprises a lipid bilayer comprising a cationic lipid and/or a functionalized lipid. In some embodiments, said functionalized lipid is a lipid functionalized with a group that can bond to a nucleic acid. In some embodiments, at least one nucleic acid is covalently bonded to the functionalized lipid or attached to the cationic lipid via electrostatic interactions. In some embodiments, the lipid bilayer comprises a mixture comprising one or more of a thiol- or dithiol-functionalized 1,2-distearoyl-sn-glycero-3-phosphoethanolamine (DSPE), 1,2-dioleoyl-3-trimethylammonium propane (DOTAP), and 1,2-dioleoyl-sn-glycero-3-phosphocholine (DOPC). In some embodiments, the at least one nucleic acid is selected from the group comprising a siRNA, a miRNA, and an AS ODN. In some embodiments, the siRNA is selected from the group comprising survivin siRNA, ERCC-1 siRNA, P-glycoprotein siRNA (P-gp siRNA), Bcl-2 siRNA, and a mixture thereof.

In some embodiments, the nanoparticle further comprises of one or more passivating agents, optionally a hydrophilic polymer; a targeting agent, optionally a RGD peptide; and an immunotherapy agent.

In some embodiments, the nanoparticle has a diameter of about 20 nanometers (nm) to about 300 nm. In some embodiments, the nanoparticle has a diameter of about 20 nm to about 200 nm. In some embodiments, the nanoparticle has a diameter of about 20 nm to about 140 nm (e.g., about 20, about 40, about 60, about 80, about 100, about 120, or about 140 nm).

In some embodiments, the nanoparticle adsorbs plasma proteins such as apolipoprotein B-100 (apo B-100) for active transport to tumors via LDL receptor-mediated endocytosis.

In some embodiments, the presently disclosed subject matter provides a pharmaceutical formulation comprising: (i) a pharmaceutically acceptable carrier and (ii) a prodrug of the presently disclosed subject matter having the structure D-BL-L as described herein above or a nanoparticle of the presently disclosed subject matter as described herein above (e.g., a core-shell nanoparticle wherein the core comprises a metal-organic framework (e.g., a NCP) and the shell comprises a lipid or lipid bilayer coating comprising a prodrug having a structure D-BL-L as described herein). In some embodiments, the pharmaceutically acceptable carrier is pharmaceutically acceptable in humans.

In some embodiments, the presently disclosed subject matter provides a method of treating cancer in a subject in need thereof (e.g., a subject who has been diagnosed with cancer or a recurrence thereof). In some embodiments, the subject is a mammal. In some embodiments, the subject is a human. In some embodiments, the presently disclosed method comprises administering to the subject a prodrug of the structure D-BL-L as described herein, a nanoparticle as described herein (i.e., a nanoparticle comprising (a) a core comprising a metal-organic matrix material, and (b) a coating layer covering at least a portion of the surface of the core, wherein the coating layer comprises a lipid layer or a lipid bilayer, wherein said lipid layer or lipid bilayer further comprises a prodrug of the formula D-BL-L as disclosed herein), or a pharmaceutically acceptable formulation of said prodrug or said nanoparticle.

In some embodiments, the method further comprises administering to the subject an additional cancer treatment. In some embodiments, the additional cancer treatment is selected from the group comprising surgery, radiotherapy, chemotherapy, toxin therapy, immunotherapy, cryotherapy and gene therapy. In some embodiments, the additional cancer treatment is immunotherapy. In some embodiments, the immunotherapy comprises administering to the subject an immunotherapy agent; optionally wherein the immunotherapy agent is selected from the group comprising an anti-CD52 antibody, an anti-CD20 antibody, an anti-CD47 antibody an anti-GD2 antibody, a cytokine, polysaccharide K; a PD-1 inhibitor, a PD-L1 inhibitor, a CTLA-4 inhibitor, an IDO inhibitor, a CCR7 inhibitor, an OX40 inhibitor, a TIM3 inhibitor, and a LAG3 inhibitor. In some embodiments, the cytokine is selected from the group comprising an interferon and an interleukin. In some embodiments, the cytokine is selected from the group comprising IFN-α, IFN-γ, IL-2, IL-12 and TNF-α.

In some embodiments, the cancer is selected from the group comprising a head tumor, a neck tumor, breast cancer, a gynecological tumor, a brain tumor, colorectal cancer, lung cancer, mesothelioma, a soft tissue sarcoma, skin cancer, connective tissue cancer, adipose cancer, lung cancer, stomach cancer, anogenital cancer, kidney cancer, bladder cancer, colon cancer, prostate cancer, central nervous system cancer, retinal cancer, blood cancer, neuroblastoma, multiple myeloma, lymphoid cancer, and pancreatic cancer. In some embodiments, the cancer is a metastatic cancer. In some embodiments, the metastatic cancer is metastatic colorectal cancer (mCRC).

In some embodiments, the method comprises administering to the subject a nanoparticle wherein the nanoparticle core comprises a metal bisphosphate or metal bisphosphonate coordination polymer comprising a multivalent metal ion (e.g., Ca²⁺, Mg²⁺, Mn²⁺, Zn²⁺ or any combination thereof) and a bisphosphate or bisphosphonate, wherein said bisphosphate or bisphosphonate is a bisphosphate or bisphosphonate prodrug of cisplatin, oxaliplatin or carboplatin; and wherein the coating layer comprises a lipid layer or lipid bilayer comprising a prodrug having the structure D-BL-L wherein D is a monovalent drug moiety of an anti-cancer drug compound; L is a monovalent lipid moiety; and BL is a bivalent linker moiety wherein D is attached to BL via a cleavable carbonate or a carbamate bond and wherein BL comprises an acetal group, wherein an oxygen atom of the acetal group is directly attached to a carbon atom of a carbonate or carbamate. In some embodiments, the nanoparticle core comprises a metal bisphosphate coordination polymer. In some embodiments, D is a monovalent derivative of an anti-cancer drug compound, e.g., a hydrophobic drug compound, such as a drug compound selected from the group comprising ET, PPX, PTX, DTX, DHA, CPT, SN38, Topotecan, Doxorubicin, Epirubicin, Idarubicin, Vincristine, Mitoxantrone, Artesunate, Capecitabine, Octreotide, Leuprolide, and Goserelin. In some embodiments, BL comprises an acetal having a structure of the formula:

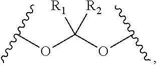

wherein R₁ and R₂ are independently selected from the group comprising H, alkyl (e.g., C₁-C₆ alkyl), substituted alkyl, aralkyl, substituted aralkyl, aryl, and substituted aryl. In some embodiments, R₁ and R₂ are selected from H, methyl, and phenyl. In some embodiments, R₁ and R₂ are the same. In some embodiments, R₁ and R₂ are different. In some embodiments, R₁ and R₂ are each H.

In some embodiments, the nanoparticle core further comprises a hydrophilic anti-cancer agent embedded therein. In some embodiments, the hydrophilic anti-cancer agent is GMP.

In some embodiments, the prodrug in the lipid bilayer is a prodrug of SN38. In some embodiments, the prodrug has a structure of the formula:

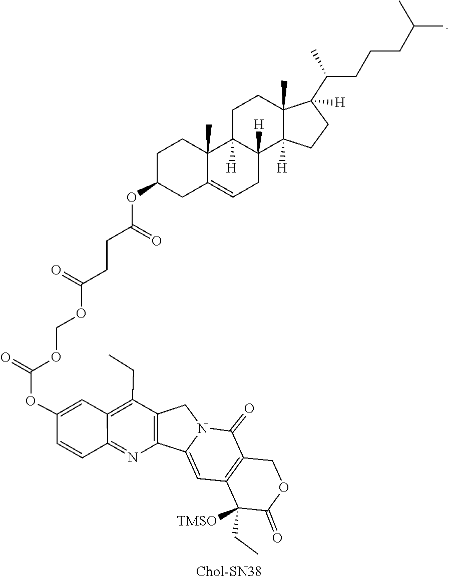

In some embodiments, the bisphosphate in the nanoparticle core is Pt(dach)(oxalate)(bisphosphoramidic acid).

In some embodiments, the method further comprises administering to the subject an immunotherapy agent. In some embodiments, the immunotherapy agent is selected from the group comprising an anti-CD52 antibody, an anti-CD20 antibody, anti-CD47 antibody, an anti-GD2 antibody, polysaccharide K and a cytokine. In some embodiments, the immunotherapy agent is selected from the group comprising a radiolabeled antibody, an antibody-drug conjugate, and a neoantigen. In some embodiments, the immunotherapy agent is selected from the group comprising Alemtuzumab, Ofatumumab, Rituximab, Zevalin, Adcetris, Kadcyla and Ontak. In some embodiments, the immunotherapy agent is selected from the group comprising a PD-1 inhibitor, a PD-L1 inhibitor, a CTLA-4 inhibitor, an IDO inhibitor, a CCR7 inhibitor, a OX40 inhibitor, a TIM3 inhibitor, and a LAG3 inhibitor. In some embodiments, the cytokine is selected from the group comprising an interferon and an interleukin. In some embodiments, the cytokine is selected from the group comprising IFN-α, IFN-γ, IL-2, IL-12 and TNF-α.

In some embodiments, administration of the nanoparticle provides at least a 2-fold increase in a tumor area under the curve (AUC) of at least one anti-cancer agent compared to administration of an equivalent amount of the at least one anti-cancer agent wherein the at least one anti-cancer agent is not associated with a nanoparticle and/or prodrug of the presently disclosed subject matter (e.g., compared to administration of free anti-cancer agent). In some embodiments, the administration of the nanoparticle provides at least a 4-fold increase in tumor AUC or a more than 4-fold increase in tumor AUC compared to administration of an equivalent amount of the at least one anti-cancer agent wherein the at least one anti-cancer agent is not associated with a nanoparticle and/or prodrug of the presently disclosed subject matter.

In some embodiments, the presently disclosed subject matter provides a method of treating cancer in a subject in need thereof wherein the method comprises administering to the subject a composition comprising a nanoscale particle comprising a lipid-conjugate prodrug comprising a cleavable carbonate linkage and a metal-organic matrix material core, optionally a NCP core. In some embodiments, the nanoscale particle further comprises a photosensitizer and the method further comprises irradiating the subject or a treatment area of the subject with radiation having a wavelength suitable to activate the photosensitizer.

### III. Formulations

Thus, the compositions of the presently disclosed subject matter comprise, in some embodiments, a composition that includes a pharmaceutically acceptable carrier. Any suitable pharmaceutical formulation can be used to prepare the compositions for administration to a subject. In some embodiments, the composition and/or carriers can be pharmaceutically acceptable in humans.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the subject; and aqueous and non-aqueous sterile suspensions that can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are sodium dodecyl sulfate (SDS), in one example in the range of 0.1 to 10 mg/ml, in another example about 2.0 mg/ml; and/or mannitol or another sugar, for example in the range of 10 to 100 mg/ml, in another example about 30 mg/ml; and/or phosphate-buffered saline (PBS).

It should be understood that in addition to the ingredients particularly mentioned above, the formulations of this presently disclosed subject matter can include other agents conventional in the art having regard to the type of formulation in question. For example, sterile pyrogen-free aqueous and non-aqueous solutions can be used.

### IV. Subjects

The methods and compositions disclosed herein can be used on a sample either in vitro (for example, on isolated cells or tissues) or in vivo in a subject (i.e., living organism, such as a patient). In some embodiments, the subject or patient is a human subject, although it is to be understood that the principles of the presently disclosed subject matter indicate that the presently disclosed subject matter is effective with respect to all vertebrate species, including mammals, which are intended to be included in the terms “subject” and “patient”. Moreover, a mammal is understood to include any mammalian species for which employing the compositions and methods disclosed herein is desirable, particularly agricultural and domestic mammalian species.

As such, the methods of the presently disclosed subject matter are particularly useful in warm-blooded vertebrates. Thus, the presently disclosed subject matter concerns mammals and birds. More particularly provided are methods and compositions for mammals such as humans, as well as those mammals of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos or as pets (e.g., parrots), as well as fowl, and more particularly domesticated fowl, for example, poultry, such as turkeys, chickens, ducks, geese, guinea fowl, and the like, as they are also of economic importance to humans. Thus, also provided is the treatment of livestock including, but not limited to domesticated swine (pigs and hogs), ruminants, horses, poultry, and the like.

### V. Administration

Suitable methods for administration of a composition of the presently disclosed subject matter include, but are not limited to intravenous and intratumoral injection, oral administration, subcutaneous administration, intraperitoneal injection, intracranial injection, and rectal administration. Alternatively, a composition can be deposited at a site in need of treatment in any other manner, for example by spraying a composition within the pulmonary pathways. The particular mode of administering a composition of the presently disclosed subject matter depends on various factors, including the distribution and abundance of cells to be treated and mechanisms for metabolism or removal of the composition from its site of administration. For example, relatively superficial tumors can be injected intratumorally. By contrast, internal tumors can be treated following intravenous injection.

In one embodiment, the method of administration encompasses features for regionalized delivery or accumulation at the site to be treated. In some embodiments, a composition is delivered intratumorally. In some embodiments, selective delivery of a composition to a target is accomplished by intravenous injection of the composition followed by photodynamic treatment (light irradiation) of the target.

For delivery of compositions to pulmonary pathways, compositions of the presently disclosed subject matter can be formulated as an aerosol or coarse spray. Methods for preparation and administration of aerosol or spray formulations can be found, for example, in U.S. Pat. Nos. 5,858,784; 6,013,638; 6,022,737; and 6,136,295.

### VI. Doses

An effective dose of a composition of the presently disclosed subject matter is administered to a subject. An “effective amount” is an amount of the composition sufficient to produce detectable treatment. Actual dosage levels of constituents of the compositions of the presently disclosed subject matter can be varied so as to administer an amount of the composition that is effective to achieve the desired effect for a particular subject and/or target. The selected dosage level can depend upon the activity (e.g., cytotoxic or PDT activity or chemotherapeutic loading) of the composition and the route of administration

After review of the disclosure herein of the presently disclosed subject matter, one of ordinary skill in the art can tailor the dosages to an individual subject, taking into account the particular formulation, method of administration to be used with the composition, and nature of the target to be treated. Such adjustments or variations, as well as evaluation of when and how to make such adjustments or variations, are well known to those of ordinary skill in the art.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Nanoscale Coordination Polymer Core-Shell Nanoparticles Codeliver Oxaliplatin and SN38

### Synthesis of cholesterol-conjugated SN38 (Chol-SN38)

Synthesis of chol-SN38 is summarized in FIG. 1. Cholesterol was coupled with succinic acid and then to p-nitrophenol as a leaving group. The hydroxyl group of SN-38 was protected by trimethylsilyl group. Then the two compounds were mixed in the presence of a base to yield Chol-SN38.

### Synthesis of chloromethyl 4-nitrophenyl carbonate

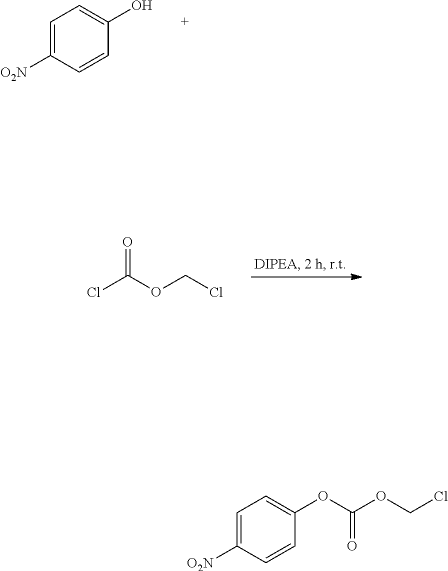 [SMILES: O=[N+]([O-])c1ccc(O)cc1 ; O=C(Cl)OCCl ; O=C(OCCl)Oc1ccc([N+](=O)[O-])cc1]

10 g (72 mmol) 4-nitrophenol was dissolved in 200 mL anhydrous dichloromethane (DCM) in a 500 mL round-bottom flask under nitrogen protection. 20 mL (115 mmol, 1.6 eq) di-isopropylethylamine (DIPEA) was then added to the flask and the flask was cooled in an ice bath. 10 mL (14.5 g, 115 mmol, 1.6 eq) chloromethyl chloroformate was added dropwise to the solution. The solution was then stirred at room temperature for 2 h. The yellowish color disappeared and the final solution turned deep red-pink after 2 h reaction. The solution was washed with 200 mL water twice, followed by 200 mL 1M HCl and another 200 mL water, then washed with 200 mL saturated NaCl. The organic phase was then dried over anhydrous Na₂SO₄ for 2 h. The solution was concentrated on a rotary evaporator to remove DCM and obtain a deep red oil as a crude product.

600 mL 10:1 hexane: isopropyl ether was added to the crude product and heated to boil to obtain a light yellow or colorless solution and deep red precipitate. The solution was transferred to another flask and cooled in −20° C. freezer overnight. Colorless or pale-yellow needle-shaped crystals formed in a freezer and were collected as the pure product. The hexane/isopropyl ether solution can be further concentrated to obtain more product. Yield: 13.5 g (58 mmol, 81%).

¹H-NMR (500 MHz, CDCl3): δ=5.85 (s, 2H), 7.42 (d, J=8 Hz, 2H), 8.30 (d, J=9 Hz, 2H). These NMR data are consistent with literature report (J. Org. Chem. 1997, 62, 5, 1356-1362).

### Synthesis of Cholesteryl Hydrogen Succinate

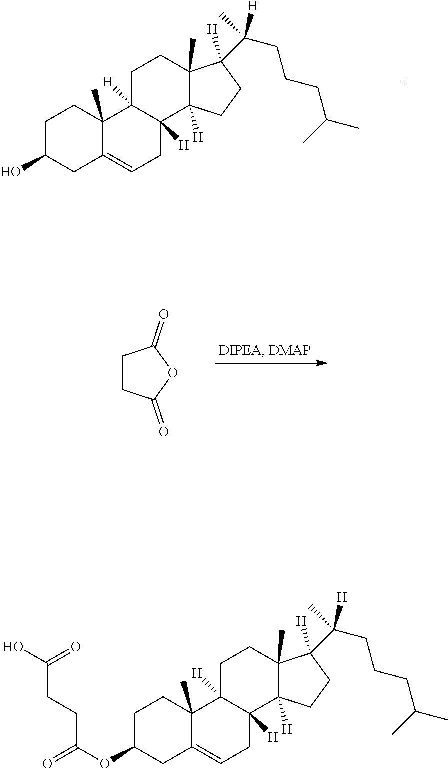 [SMILES: CC(C)CCC[C@@H](C)[C@H]1CC[C@H]2[C@@H]3CC=C4C[C@@H](O)CC[C@]4(C)[C@H]3CC[C@]12C ; O=C1CCC(=O)O1 ; CC(C)CCC[C@@H](C)[C@H]1CC[C@H]2[C@@H]3CC=C4C[C@@H](OC(=O)CCC(=O)O)CC[C@]4(C)[C@H]3CC[C@]12C]

8 g (20 mmol) cholesterol, 6 g succinic anhydride (60 mmol, 3 eq), 400 mg N,N-dimethylpyridine (DMAP, 3.25 mmol as catalyst), 6 mL DIPEA (35 mmol, 1.75 eq) were mixed in 250 mL anhydrous THF and refluxed under nitrogen for 48 h to obtain a deep yellow/red solution. Then all solvents were evaporated on a rotary evaporator to obtain a white-brown solid as a crude product.

The crude product was transferred to a 500 mL flask using 100 mL tetrahydrofuran (THF). 150 mL saturated NaHCO₃ solution was added to the flask and the mixture was stirred for 3 h until no more bubble was generated. Then the mixture was neutralized with 1M HCl until pH<5 to obtain a light brown liquid-solid mixture with bubbles. The mixture was extracted with 200 mL ethyl acetate (EtOAc) three times and the combined organic phase was further washed by 200 mL 1M HCl twice and 200 mL saturated NaCl. The EtOAc solution was dried over anhydrous Na₂SO₄ for 2 h and evaporated to obtain a light brown solid. The solid was dissolved in 100 mL boiling EtOAc and cooled in −20° C. freezer overnight. White crystals formed and were collected as the pure product. Yield: 9.8 g (20 mmol, 100%).

¹H-NMR (500 MHz, CDCl3): δ=0.5-2.35 (m, 43H), 2.62 (t, 2H), 2.69 (t, 2H), 4.63 (m, 1H), 5.38 (d, 1H, J=4.4 Hz). HRMS: m/z=504.4040 ([M+NH₄]⁺). These spectroscopic data are consistent with literature report (J Med. Chem. 2010, 53, 21, 7632-7638).

### Synthesis of Iodomethyl 4-Nitrophenyl Carbonate

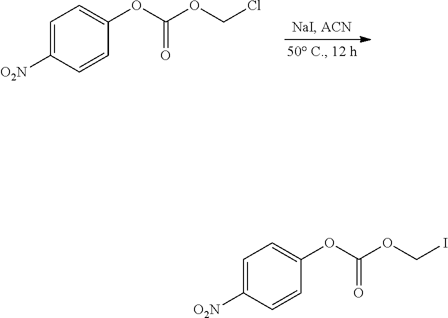 [SMILES: O=C(OCCl)Oc1ccc([N+](=O)[O-])cc1 ; O=C(OCI)Oc1ccc([N+](=O)[O-])cc1]

7 g (30 mmol) chloromethyl 4-nitrophenyl carbonate an 14 g (90 mmol, 3 eq) NaI were dissolved in 300 mL anhydrous acetonitrile and stirred at 50° C. under nitrogen for 24 h. The solution turned light yellow and white precipitates (NaCl) formed. The solvent was removed with a rotary evaporator and the solid was further dried under vacuum for 2 h. 300 mL DCM was added to the dry solid to extract the product. The DCM solution was filtered to remove NaCl and NaI solids and concentrated to obtain a light-yellow oil or solid as a crude product.

The crude product was used in the following steps without further purification.

### Synthesis of 20-O-Trimethylsilyl SN38

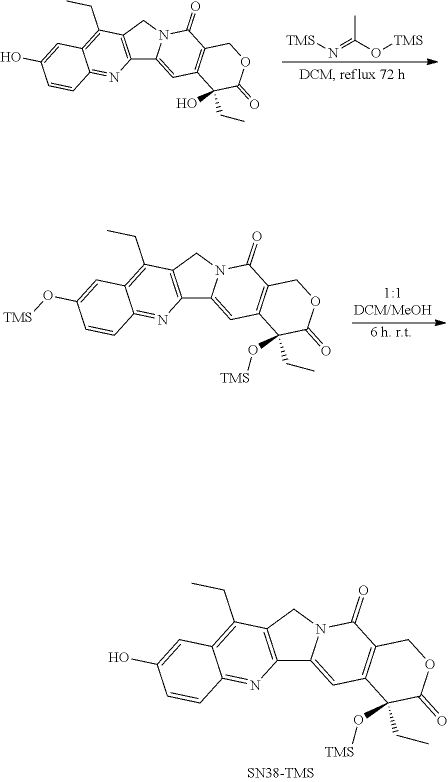 [SMILES: CCc1c2c(nc3ccc(O)cc13)-c1cc3c(c(=O)n1C2)COC(=O)[C@]3(O)CC ; C/C(=N\[Si](C)(C)C)O[Si](C)(C)C ; CCc1c2c(nc3ccc(O[Si](C)(C)C)cc13)-c1cc3c(c(=O)n1C2)COC(=O)[C@@]3(CC)O[Si](C)(C)C ; CCc1c2c(nc3ccc(O)cc13)-c1cc3c(c(=O)n1C2)COC(=O)[C@@]3(CC)O[Si](C)(C)C]

5 g (12.7 mmol) SN38 was mixed with 25 mL anhydrous DCM and 25 mL N,O-bis(trimethylsilyl)acetamide. The mixture was refluxed under nitrogen for 72 h. SN38 gradually dissolved during the reaction to form a deep red solution. The reaction was monitored by TLC (3:1 DCM: EtOAc, visible under UV light) to make sure all SN38 was converted to bis-TMS-SN38. The solution was dried using a rotary evaporator and then further dried under vacuum. The brown solid (bis-TMS-SN38) was dissolved in 100 mL DCM and 100 mL methanol and stirred at room temperature for 6 h and the reaction was monitored by TLC until all bis-TMS-SN38 was converted to 20-O-TMS-SN38. Then the solution was dried to obtain a deep yellow/brown solid as a crude product. The crude product was further dried under high vacuum to remove methanol and then directly used in the following steps without further purification. ¹H NMR (500 MHz, Chloroform-d) δ 9.74 (s, 1H), 8.13 (d, J=9.1 Hz, 1H), 7.57 (s, 1H), 7.51 (dd, J=9.1, 2.6 Hz, 1H), 7.47 (d, J=2.6 Hz, 1H), 5.70 (d, J=16.4 Hz, 1H), 5.28 (d, J=16.3 Hz, 1H), 5.23 (s, 2H), 3.05 (d, J=7.6 Hz, 2H), 1.85 (dd, J=7.3, 2.3 Hz, 2H), 1.32 (t, J=7.6 Hz, 3H), 0.88 (t, J=7.3 Hz, 3H), 0.21 (s, 11H). ¹³C NMR (126 MHz, CDCl3) δ 172.26, 157.87, 156.81, 152.37, 148.83, 146.71, 144.42, 143.87, 131.68, 129.00, 128.77, 126.95, 122.98, 117.92, 105.61, 98.53, 77.33, 77.07, 76.82, 75.90, 65.84, 49.57, 32.55, 23.20, 13.60, 7.78, 2.15, 1.91, 1.67. HRMS: m/z=465.1850 (expected 465.1767 for [M+H]⁺).

### Synthesis of Cholest-5-en-3-ol ((((4-nitrophenoxy)carbonyl)oxy)methyl) succinate cholesterol linker)

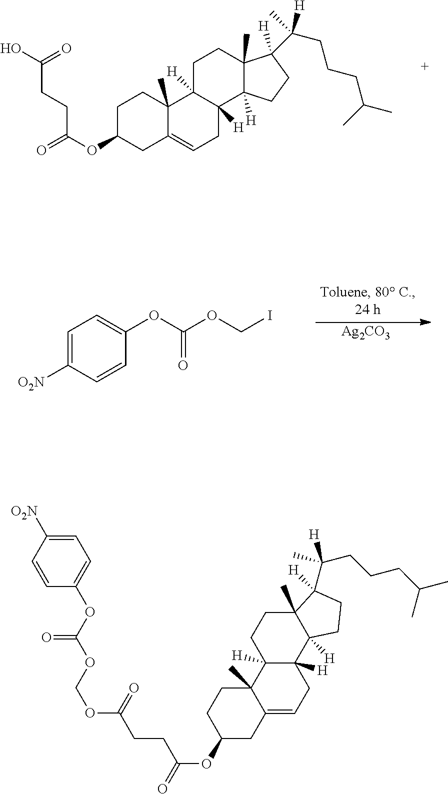

Crude iodomethyl 4-nitrophenyl carbonate (from 7 g, 30 mmol chloromethyl 4-nitrophenyl carbonate, 1.5 eq) was dissolved in 250 mL anhydrous toluene and then 9.8 g (20 mmol, 1 eq) cholesteryl hydrogen succinate was added to the solution. The mixture was stirred with heating until all solids dissolved. 5.6 g (20 mmol, 1 eq) Ag₂CO₃ was then added to the solution and the solution was stirred in dark at 80° C. under nitrogen protection for 24 h. The resulting pale-yellow solution with black precipitates was filtered through celite and concentrated to obtain a light-yellow oil. The product was further purified by column chromatography using silica. 30% hexanes in DCM was used to elute the first two spots and then changed to pure DCM to separate the product. Yield: 7 g (10 mmol, 50%). ¹H NMR (500 MHz, Chloroform-d) δ 8.32-8.27 (m, 2H), 7.47-7.41 (m, 2H), 5.90 (s, 2H), 5.35 (dt, J=5.3, 1.7 Hz, 1H), 4.66-4.57 (m, 1H), 2.75 (ddd, J=7.3, 5.8, 1.2 Hz, 2H), 2.67 (ddd, j=7.3, 6.0, 1.2 Hz, 2H), 2.32 (dd, J=7.3, 1.8 Hz, 2H), 2.05-1.92 (m, 2K), 1.89-1.80 (m, 3H), 1.65-0.79 (i, 40H), 0.68 (s, 3H). ¹³C NMR (126 MHz, CDCl₃) δ 171.27, 170.91, 155.08, 151.45, 145.64, 139.48, 125.39, 122.83, 121.78, 82.49, 77.34, 77.09, 76.83, 74.69, 56.66, 56.14, 50.01, 42.32, 39.72, 39.55, 38.03, 36.97, 36.59, 36.21, 35.82, 31.91, 31.86, 29.13, 28.99, 28.26, 28.04, 27.72, 24.31, 23.87, 22.87, 22.61, 21.05, 19.31, 18.75, 11.88. ESI-MS: m/z=699.4226 (expected 699.4221 for [M+NH₄]⁺).

### Synthesis of 7-ethyl-10-(((((cholest-5-en-3-oxy)-4-oxobutanoyl)oxy)methoxy)-carbonyl)oxyl-20-O-trimethylsilyl camptothecin (Chol-SN38)

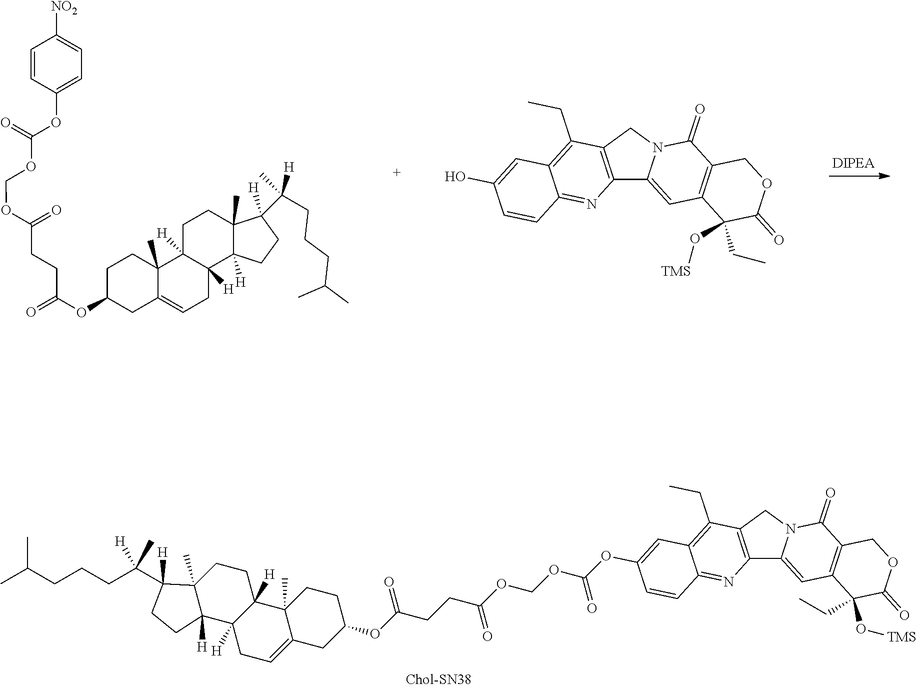 [SMILES: CC(C)CCC[C@@H](C)[C@H]1CC[C@H]2[C@@H]3CC=C4C[C@@H](OC(=O)CCC(=O)OCOC(=O)Oc5ccc([N+](=O)[O-])cc5)CC[C@]4(C)[C@H]3CC[C@]12C ; CCc1c2c(nc3ccc(O)cc13)-c1cc3c(c(=O)n1C2)COC(=O)[C@@]3(CC)O[Si](C)(C)C ; CCc1c2c(nc3ccc(OC(=O)OCOC(=O)CCC(=O)O[C@H]4CC[C@@]5(C)C(=CC[C@H]6[C@@H]7CC[C@H]([C@H](C)CCCC(C)C)[C@@]7(C)CC[C@@H]65)C4)cc13)-c1cc3c(c(=O)n1C2)COC(=O)[C@@]3(CC)O[Si](C)(C)C]

7 g (10 mmol) cholesterol linker and crude 20-O-TMS-SN38 from 5 g SN38 (12.7 mmol, 1.27 eq) was dissolved in 250 mL anhydrous DCM. 10 mL (60 mmol, 6 eq) DIPEA was added to the solution and the solution was stirred at room temperature under nitrogen protection for 24 h. The resulting deep red solution was diluted with 500 mL DCM and washed with saturated NaHCO₃ three times, followed by 1M HCl and saturated NaCl. The organic layer was then dried over anhydrous Na₂SO₄ for 2 h and concentrated by rotary evaporator. The product was further purified by column chromatography using silica. The product was eluted with 15:1 DCM: EtOAc. Yield: 8 g (8 mmol, 80%). ¹H NMR (500 MHz, Chloroform-d) δ 8.28 (d, J=9.2 Hz, 1H), 7.96 (d, J=2.6 Hz, 1H), 7.68 (dd, J=9.2, 2.6 Hz, 1H), 7.52 (s, 1H), 5.93 (s, 2H), 5.68 (d, J=16.6 Hz, 1H), 5.34-5.19 (m, 5H), 4.61 (dt, J=7.8, 2.2 Hz, 1H), 3.16 (d, J=7.7 Hz, 2H), 2.79-2.73 (m, 2H), 2.70-2.63 (m, 2H), 2.30 (d, J=7.7 Hz, 2H), 2.02-1.73 (m, 8H), 1.63-0.74 (m, 50H), 0.63 (s, 3H). ¹³C NMR (126 MHz, CDCl3) δ 171.92, 171.24, 170.93, 157.59, 152.34, 152.28, 151.76, 149.47, 147.58, 146.26, 145.47, 139.43, 132.45, 127.43, 127.35, 124.34, 122.74, 119.00, 114.08, 98.27, 82.44, 77.30, 77.05, 76.79, 75.82, 74.64, 65.93, 56.65, 56.16, 49.96, 49.32, 42.27, 39.71, 39.49, 38.00, 36.92, 36.53, 36.16, 35.79, 32.75, 31.84, 31.79, 29.70, 29.13, 29.01, 28.22, 28.01, 27.70, 24.26, 23.88, 23.21, 22.83, 22.57, 20.98, 19.28, 18.70, 14.02, 11.83, 7.89, 2.11, 1.87, 1.63. ESI-MS: m/z=1007.5456 (expected 1007.5375 for [M+NH₄]⁺).

### Synthesis and Characterization of OxPt SN38 Core-Shell Nanoparticle

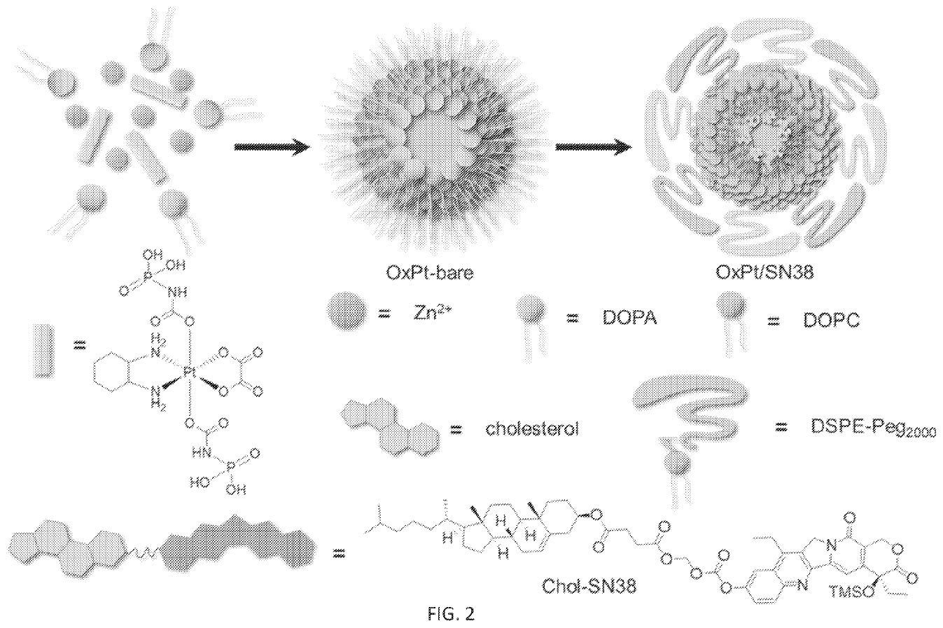

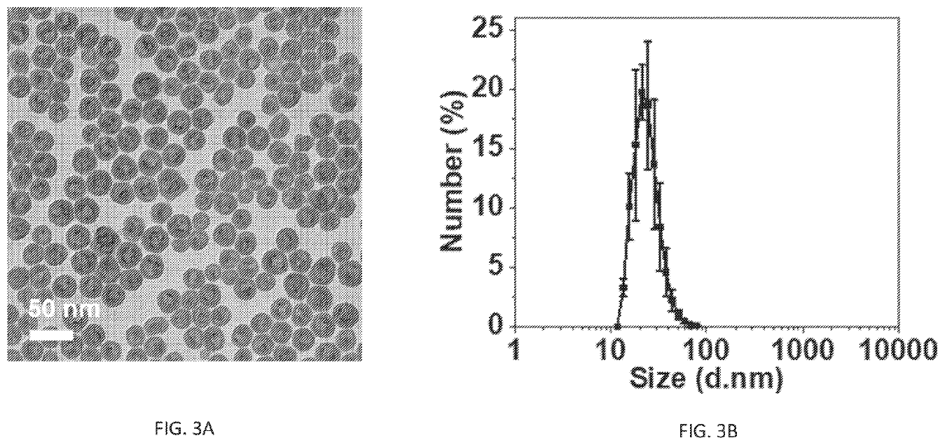

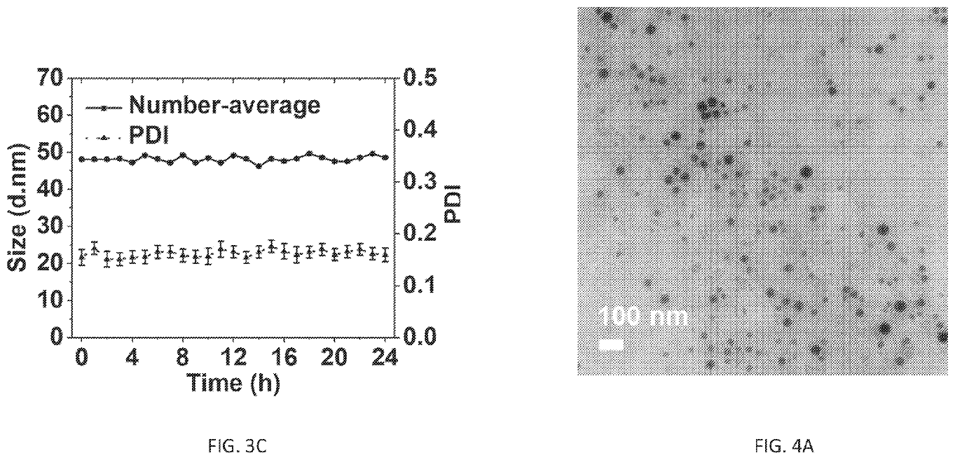

The potent topoisomerase 1 inhibitor SN38 was conjugated to cholesterol via an acid-sensitive and enzymatically cleavable acetal linker to form Chol-SN38. A bulky but acidic-sensitive trimethylsilyl (TMS) group was added to Chol-SN38 at the 0-20 position of SN38 to disrupts strong π-π stacking of two-dimensional SN38 moieties and drive the formation of stable lipid coating on the NCP core. The core-shell NCP particle OxPt/SN38 was prepared in two steps. See FIG. 2. First, Pt(dach)(oxalate)(bisphosphoramidic acid) (OxPt-bp) (Duan, X.; et al. Nat. Commun. 2020, 10: 1899) was first co-polymerized with Zn²⁺ ions in the presence of DOPA in a reverse microemulsion containing Triton X-100/hexanol/cyclohexane to form DOPA-capped bare NCP particles (OxPt-bare). OxPt-bare particles were monodispersed in tetrahydrofuran as observed under transmission electron microscopy (TEM). See FIG. 3A. Dynamic light scattering (DLS) measurements showed a Z-averaged diameter of 49.0±3.0 nm and a polydispersity index (PDI) of 0.17±0.03 for OxPt-bare. See FIG. 3B. OxPt-bare showed high stability with no change of particle size and PDI in tetrahydrofuran at room temperature over a 24 h period. See FIG. 3C.

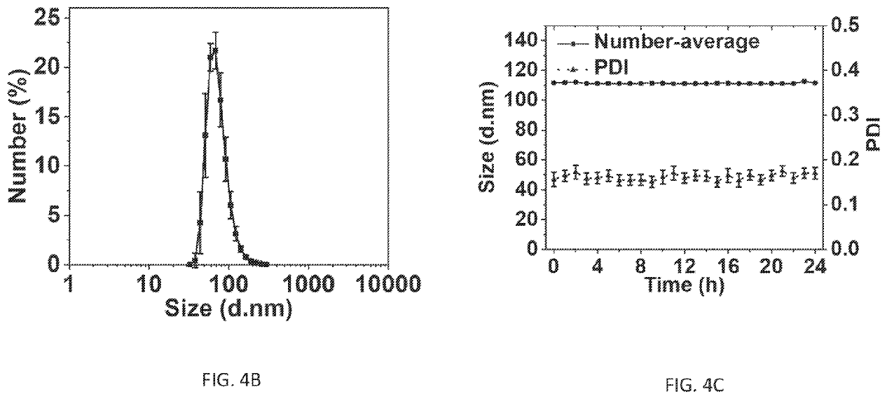

Chol-SN38 was then incorporated into the lipid layer together with cholesterol, DOPC and DSPE-PEG₂₀₀₀ on the surface of OxPt-bare to form core-shell NCP particle OxPt/SN38. 0.21 mg cholesterol, 0.42 mg DOPC, 0.75 mg DSPE-PEG2k, 0.2 mg chol-SN38 and 0.5 mg OxPt bare NCP particle (Duan, X.; et al. Nat. Commun. 2020, 10: 1899) was mixed in 80 uL THF and added to 500 μL 30% EtOH with 1700 rpm stirring at 50° C. The mixture was concentrated to 100 μL to obtain OxPt/SN38 core-shell nanoparticle. OxPt/SN38 particles were monodispersed in aqueous solutions as observed under TEM. See FIG. 4A. Z-average diameter and PDI of the OxPt/SN38 particle were 111.6 nm and 0.138 by DLS. See FIG. 4B. OxPt/SN38 particles were stable in PBS with 5 mg/mL bovine serum albumin (BSA) with no changes in size or PDI at 37° C. for 24 h. See FIG. 4C.

### OxPt SN38 Transfers Chol-SN38 to LDL

LDL is the key transporter of cholesterol and cholesterol esters to peripheral cells. It was hypothesized that LDL would strongly bind to Chol-SN38 to enhance its delivery to tumors via LDLR-mediated endocytosis. Isothermal titration calorimetry (ITC) measurements showed strong binding of Chol-SN38 to LDL with a binding constant (Kₐ) of (4.97±0.24)×10⁵ M⁻¹, which is >2000 times higher than the binding affinity between Chol-SN38 and albumin. See Table 1, below. Interestingly, titration of LDL with OxPt/SN38 also led to exothermic binding with an apparent Kₐ of (3.34±1.30)×10⁴ M⁻¹. OxPt/SN38 showed low affinity with albumin with an apparent Kₐ of 27.5±1.8 M⁻¹. See Table 1, below. It is believed that the apparently high affinity of OxPt/SN38 to LDL is due to the transfer of chol-SN38 from OxPt/SN38 to LDL.

TABLE 1
Binding constants of Chol-SN38 and particles to plasma proteins.

| Drugs | Proteins | Binding affinity Kₐ (10⁴ M⁻¹) |
|---|---|---|
| OxPt/SN38 | LDL | 3.34 ± 1.30 |
| OxPt/SN38 | Albumin | 0.00275 ± 0.00018 |
| Chol-SN38 | LDL | 49.7 ± 2.43 |
| Chol-SN38 | Albumin | 0.0225 ± 0.0144 |
| OxPt NCP w/o Chol | LDL | 0.284 ± 0.020 |
| Chol-SN38 micelle | LDL | 2.76 ± 0.31 |

Apparent LDL binding affinity of two control particles, OxPt NCP without chol and Chol-SN38 micelle (without the NCP core) was determined to understand which components of the particles bind to LDL. While Chol-SN38 micelle particles showed almost the same binding affinity to LDL as OxPt/SN38 particles, OxPt NCP without Chol showed one order of magnitude lower binding affinity to LDL. See Table 1, above. These results support the notion that Chol and Chol-SN38 can be transferred from NCP and micelle particles to LDL.

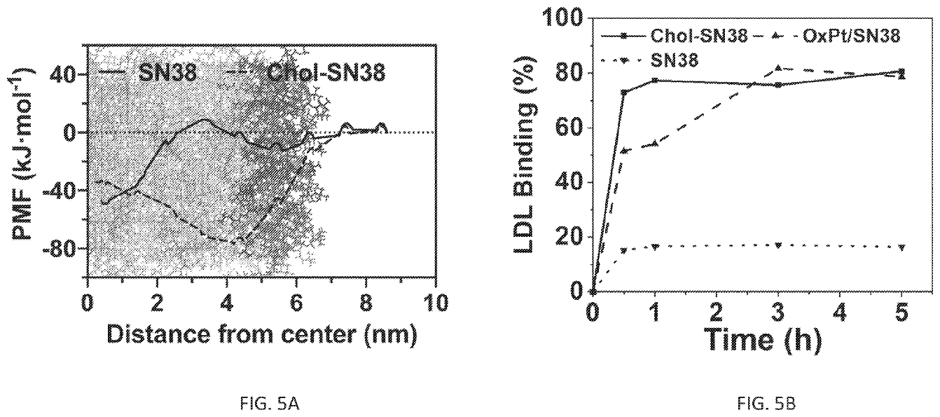

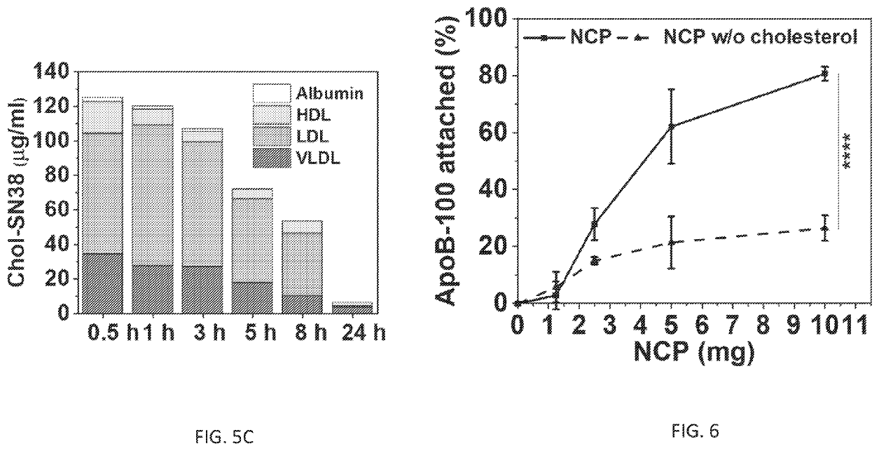

Molecular dynamics (MD) simulations were performed using a slice of the spherical LDL particle with 10% of its volume to elucidate atomic-level interactions between LDL and Chol-SN38 or SN38. The potential of mean force for transferring Chol-SN38 or SN38 from bulk water to the lipid core of LDL was computed. The potential of mean force for Chol-SN38 significantly decreased at almost every location in LDL and decreased the most by ˜80 KJ/mol at the interface of the hydrophobic core and hydrophilic shell of POPC and lyso PC (˜4 nm from the center of the hydrophobic core). See FIG. 5A. In contrast, the potential of mean force for SN38 did not decrease until approaching the center of the hydrophobic core (<2 nm) of LDL. The MD simulation results thus confirm strong attractive hydrophobic/hydrophobic interactions between Chol-SN38 and the LDL core.

The binding kinetics of Chol-SN38, OxPt/SN38, and SN38 to LDL in rat plasma at 37° C. were then determined. While Chol-SN38 quickly bound to LDL the plasma and reached an equilibrium with 74.5±3.6% chol-SN38 binding to LDL within 1 h (see FIG. 5B), OxPt/SN38 showed slower transfer of chol-SN38 to LDL but reached a similar comparable 78.6±3.1% Chol-SN38 binding to LDL in 3 h. In contrast, SN38 showed only 17.1±1.5% binding to LDL. These results demonstrate the strong binding of Chol-SN38 to LDL and transfer of Chol-SN38 from OxPt/SN38 to LDL in plasma.

The distributions of SN38, Chol-SN38, and OxPt/SN38 in various lipoproteins after incubation in rat plasmas at 37° C. for 3 h were quantified by LC-MS. Lipoproteins were separated based on their densities by NaBr gradient ultracentrifugation. SN38 was mainly distributed in the albumin fraction (69%) and only slightly distributed in the LDL fraction (17%) and HDL (14%), while Chol-SN38 mostly distributed to the LDL fraction (86%) with less than 1% Chol-SN38 in the albumin fraction, 9% Chol-SN38 in VLDL, and 5% in HDL. Interestingly, Chol-SN38 in OxPt/SN38 was efficiently transferred to lipoproteins, with 74% in LDL, 19% in VLDL, and 7% in HDL. Less than 1% of Chol-SN38 in OxPt/SN38 was observed in the albumin fraction. It is possible that some OxPt/SN38 particles are fractionated into the VLDL fraction due to the ultracentrifugation procedure.

In vivo pharmacokinetics of OxPt/SN38 on SD rats showed long blood circulation of Chol-SN38 with a half-life (t_{1/2}) of 9.7±1.0 h and an area under curve (AUC_{0→t}) of 1874.6±44.9 μg/ml*h. See FIG. 5C. The distribution of Chol-SN38 in different plasma proteins at each time point was assayed and an AUC_{0→t} of 871.6±30.8 μg/ml*h was found for LDL-bound Chol-SN38, which represents 46.5±0.5% of the total AUC_{0→t}. See FIG. 5C. LDL thus significantly contributes to the prolonged circulation of Chol-SN38 in OxPt/SN38 particles. ITC analysis, MD simulations, lipoprotein fractionation, and in vivo PK studies together show strong binding of Chol-SN38 to LDL and the efficient transfer of Chol-SN38 from OxPt/SN38 particles to LDL in plasma, suggesting the potential of hijacking LDL for enhanced delivery of highly lipophilic drugs and prodrugs to tumors.

### LDLR-Mediated Endocytosis Determines OxPt SN38 Uptake by Tumor Cells

Apo B-100 protein in LDL is a strong ligand for LDLR and responsible for efficient transfer of cholesterol to peripheral cells via LDLR-mediated endocytosis. To confirm the ability of NCPs to adsorb Apo B-100, ZnP NCPs were incubated with Apo B-100 protein for 3 h. Unbound Apo B-100 was precipitated by 0.01 M acetic acid while NCP-bound Apo B-100 remained in solution. After centrifugation, the amount of Apo B-100 in the supernatant was measured by BCA assay. As NCP concentration increased, the amount of NCP-bound Apo B-100 increased. See FIG. 6. 80.7±2.5% of Apo B-100 was captured by 10 mg ZnP particles. The Apo B-100 binding capacity decreased to 26.4±4.5% for ZnP particle without cholesterol. These results support the role of cholesterol in mediating NCP binding to Apo B-100 and its potential active uptake to tumors.

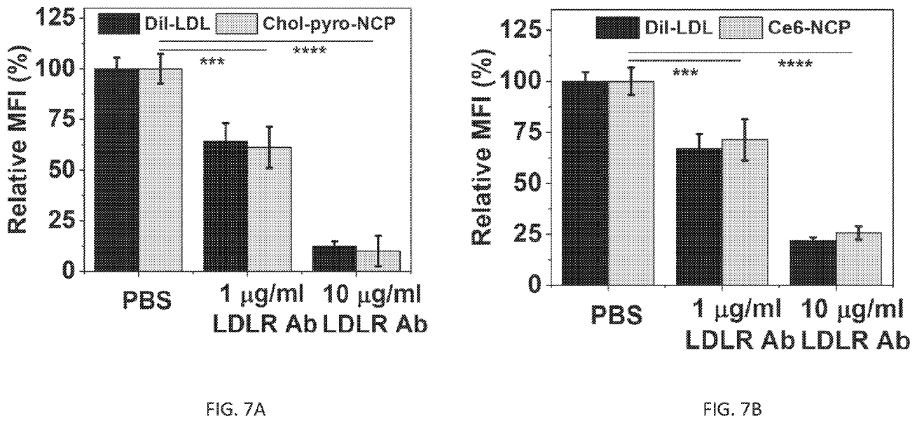

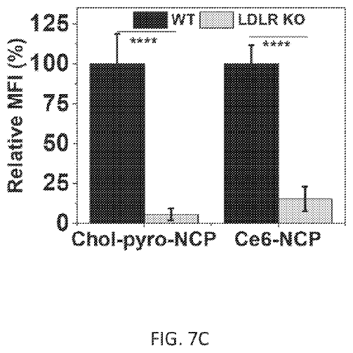

The uptake of NCP particles by tumor cells via LDLR-mediated endocytosis was confirmed using fluorescently labeled LDL (Dil-LDL) and NCP particles with Chol-pyro as a surrogate for Chol-SN38 in the shell and Ce6 as a surrogate for OxPt in the core. The uptake levels of both Chol-pyro NCP and Ce6 NCP by MC38 cells decreased with LDLR blockade by an anti-LDLR antibody (a-LDLR) in a dose-proportional manner. See FIGS. 7A and 7B. With LDLR blockade, Dil-LDL uptake by MC38 cells decreased in the same percentage as Chol-pyro NCP and Ce6 NCP, suggesting that LDLR-mediated endocytosis plays a major role in cellular uptake of NCPs. Compared to wildtype MC38 cells, LDLR knockout (KO) MC38 cells showed much lower uptake of Chol-pyro NCP (5%) and Ce6 NCP (15%). See FIG. 7C. These results suggest that LDLR-mediated endocytosis plays a major role in cellular uptake of NCPs.

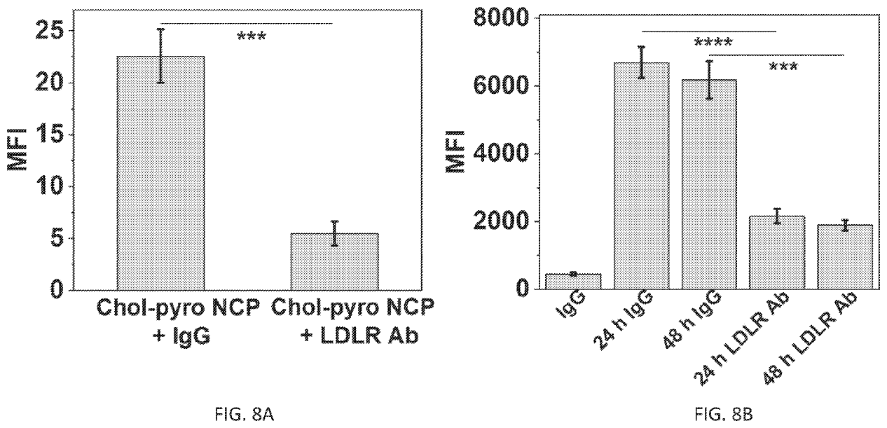

Co-localization of Chol-pyro NCP and LysoTracker and cellular uptake levels of Chol-pyro were visualized and determined by CLSM. See FIG. 8A. After 24 h incubation, Chol-pyro (red) colocalized with endo/lysosomes with Pearson's R value=0.91.

To investigate drug accumulation in tumors by LDLR-mediated endocytosis, Chol-pyro fluorescence signals of tumor slices from MC38-bearing C57BL/6 mice 24 h or 48 h after intravenous injection of 200 μg of Chol-pyro NCP with and without intratumoral injection of 1 μg a-LDLR were determined. The administration of a-LDLR decreased chol-pyro signals by 52.9±1.5% and 60.2±6.2% at 24 and 48 h time points, respectively. The tumors were also digested into single cell suspensions for flow cytometric analysis of intracellular chol-pyro signals as a function of LDLR blocking. Flow cytometric results showed that Chol-pyro levels decreased by 67.7±1.1% and 69.0±0.4% in tumor cells with LDLR blocking at 24 and 48 h time points, respectively. See FIG. 8B.

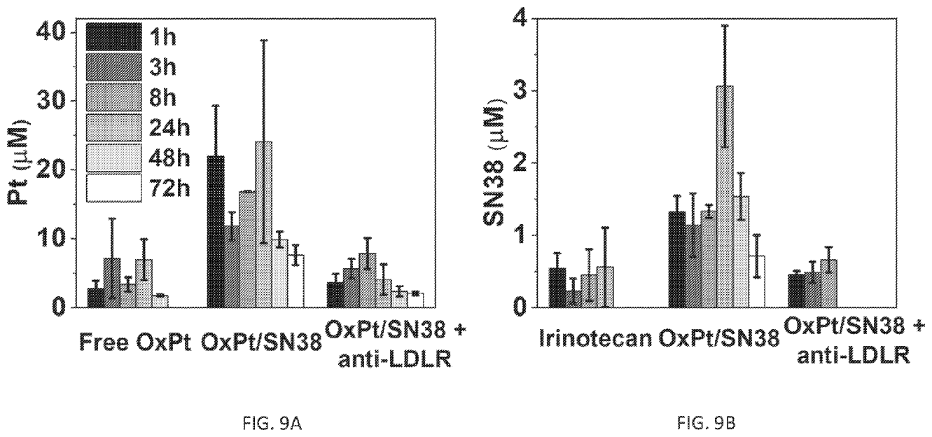

Drug accumulation in MC38 tumors were quantitatively determined following intravenous injection 3.5 mg/kg OxPt/SN38 (based on OxPt equivalents) with and without concurrent intratumoral injection of 1 μg a-LDLR. See FIGS. 9A and 9B. Without LDLR blocking, OxPt/SN38 exhibited an Pt AUC_{0→t} of 290.3±13.4 h g/mL and an SN38 AUC_{0→t} of 50.8±4.2 h g/mL in the tumors, which are 4.9 times that of free OxPt and 6 times that of free irinotecan at equivalent OxPt and/or SN38 doses. With LDLR blocking, the OxPt AUC_{0→t} decreased by 72% and the SN38 AUC_{0→t} decreased by 90%, affording a comparable level of drug accumulation to OxPt and irinotecan, respectively. While OxPt/SN38 maintained intratumoral drug concentrations above IC₅₀ values of various colon cancer cell lines (CT26, MC38, HT29, HCT116 and SW480) for 72 h, OxPt/SN38 with LDLR blocking or free drugs failed to maintain intratumoral drug concentrations above IC₅₀ values beyond 24 h. These results demonstrate that OxPt/SN38 significantly increases intratumoral OxPt and SN38 concentrations by targeting the LDLR through adsorption of Apo B-100 onto the NCP particle and transferring chol-SN38 to LDL in vivo.

### In Vitro Cytotoxicity of OxPt-SN38 in Colon Cancer Cell Lines:

Murine colorectal cancer CT26 and MC 38 cells were seeded into 96-well plates at 2500 cells/well for 24 h. Oxaliplatin, SN38, irinotecan, SN38-TMS, chol-SN38, OxPt/SN38 at various concentrations was dosed to each well and the cells were incubated for another 48 h. The cell viability was measured by ITS assay.

As listed in Table 2, below, both OxPt and SN38 have high toxicity to CT26 and MC38 cells, while irinotecan is less toxic with an IC₅₀ at nearly 80 uM. Chol-SN38 and SN38-TMS are less toxic than SN38 since they need to release SN38 to exert toxicity. OxPt/SN38 particle showed low IC₅₀ value, indicating that there is a synergistic effect between OxPt and chol-SN38 on the particle.

TABLE 2
OxPt and SN38 IC₅₀ values (μM) in CT26 and MC38 cells.

| | CT26 | MC38 |
|---|---|---|
| OxPt | 9.11 ± 0.72 | 11.37 ± 1.68 |
| SN38 | 0.22 ± 0.05 | 0.24 ± 0.08 |
| Irinotecan | 79.38 ± 6.94 | 88.38 ± 7.48 |
| SN38-TMS | 2.52 ± 0.46 | 3.04 ± 0.40 |
| Chol-SN38 | 7.41 ± 1.03 | 10.25 ± 1.99 |
| OxPt/SN38 | 1.96 ± 0.20 | 2.74 ± 0.44 |
| | (6.47 ± 0.61)^{a} | (9.26 ± 1.49)^{a} |

^{a}The numbers in parentheses refer to SN38 IC₅₀ values.

OxPt/SN38 showed similar synergistic cytotoxicity in HT29, HCT116 and SW480 human CRC cells. OxPt/SN38 was also more cytotoxic than either OxPt or irinotecan. Chol-SN38 was 5-10 times more cytotoxic than irinotecan but less cytotoxic than SN38 due to the slow release of SN38 via acid- and esterase-triggered hydrolysis processes. Interestingly, although SN38-TMS showed potent cytotoxicity with IC₅₀ values of 8.01±1.73 and 7.60±0.20 μM for CT26 and MC38 cells, respectively, this cytotoxicity likely came from SN38 generated from hydrolysis of SN38-TMS in situ as analogous SN38 derivates 20-O-tert-butyl-SN38 (SN38-Bu) and 20-O-Boc-SN38 (SN38-Boc) showed no cytotoxicity at concentrations up to 300 μM.

Apoptosis/necrosis analysis was also performed on OxPt and/or SN38 treated cells by flow cytometry. OxPt/SN38 particle treated cells showed much higher percentage of apoptosis (47.8% compared to 12.1% and 34.8% for OxPt and SN38, respectively), which supports the synergistic effect between OxPt and chol-SN38 on the particle. The similar trend was also found in the free drug groups, which supports the release of two synergistic drugs from the combination nanoparticle in vitro.

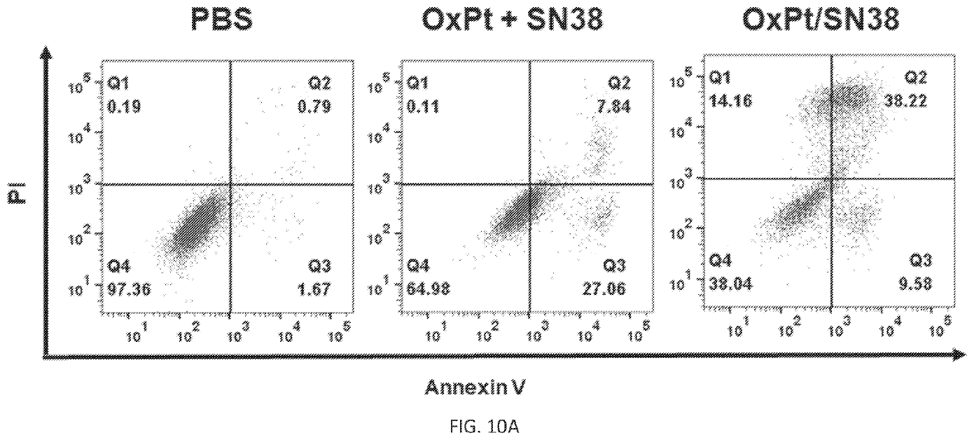

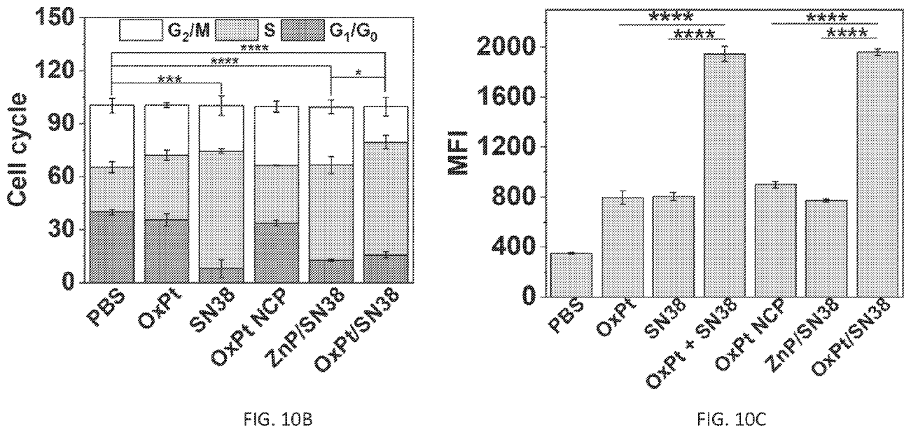

The mechanism of OxPt/SN38 induced cell death was evaluated with Annexin V-FITC staining for cell apoptosis and PI staining for cell necrosis. See FIG. 10A. Both OxPt and SN38 induced programmed cell death by apoptosis/necrosis. The combination of OxPt and SN38 increased early apoptotic Annexin V+/PI− cells (28.4±1.2% compared to 8.0±0.6% for OxPt and 25.7±1.5% for SN38). Similarly, OxPt/SN38 increased the percentage of late apoptotic/necrotic Annexin V+/PI+ cells (34.5±3.8% compared to 4.9±0.6% for OxPt NCP and 16.4±0.9% for ZnP/SN38, respectively).

Cell cycle distribution was analyzed for treated MC38 cells to evaluate DNA damage. MC38 cells treated with OxPt NCP and ZnP/SN38 showed 32.6±0.2% and 53.9±4.9% S-phase arrest, respectively. See FIG. 10B. In comparison, 25.3±3.1% cells were in S-phase for PBS control. OxPt/SN38 showed a stronger S-phase arrest of 63.8±3.8%. OxPt/SN38 particles thus effectively caused DNA damage and inhibited DNA replication for anti-proliferative effects.

Flow cytometry analysis with JC-1 staining showed that treatment of MC38 cells with OxPt/SN38 for 24 h resulted in five-fold increase in the depolarization of mitochondrial membrane potential compared to PBS control. See FIG. 10C. Mitochondrial membrane integrity loss releases cytochrome c into the cytosol for activation of caspase-9 and caspase-3 to induce apoptosis. Mitochondria and cytochrome c of treated MC38 cells were stained with a dye sold under the tradename MITOTRACKER™ (Molecular Probes, Eugene, Oregon, United States of America) (red fluorescence) and FITC-conjugated anti-cytochrome c antibody (green fluorescence), respectively. CLSM imaging showed that OxPt/SN38 treatment induced obvious separation of MITOTRACKER™ dye and anti-cytochrome c antibody signals with a Pearson's R value of 0.27, indicating significant release of cytochrome c from mitochondria.

Release of SN38 from Chol-SN38 and SN38-TMS:

To confirm that chol-SN38 loaded on OxPt/SN38 particle can release SN38 in vivo, OxPt/SN38 particle was diluted in PBS or rat plasma to make a 200 ppm solution of chol-SN38 and the solution was incubated at 37° C. Samples were collected at 1 h, 5 h and 24 h and extracted with ethyl acetate. The organic layer was analyzed using LC-HRMS to determine the percentage of SN38, TMS-SN38, and chol-SN38. When incubated in PBS, no significant release of SN38 was found after 24 h, while about 53.86% chol-SN38 decomposed into TMS-SN38 or SN38 with the help of esterase in rat plasma. See Table 3, below. This experiment proves SN38-TMS and SN38 can be released in vivo.

TABLE 3
Release of SN38-TMS and SN38 from OxPt/SN38 Particles.

| | 1 h | | 5 h | | 24 h | |
|---|---|---|---|---|---|---|
| | | Rat | | Rat | | Rat |
| | PBS | Plasma | PBS | Plasma | PBS | Plasma |
| SN38 | 1.74 | 6.03 | 3.21 | 8.02 | 3.22 | 8.38 |
| SN38-TMS | 0.81 | 15.45 | 3.60 | 20.58 | 4.16 | 45.48 |
| Chol-SN38 | 97.45 | 78.52 | 93.20 | 71.40 | 92.63 | 46.14 |

SN38-TMS was incubated in acidic conditions mimicking endosomes at 37° C. At pH=5.5, 70% SN38-TMS was converted to SN38 in 24 h and 92% 70% SN38-TMS was converted to SN38 in 48 h. This result shows that when uptaken into endosomes, SN38-TMS can be easily converted to SN38 due to low pH in endosomes. The release of SN38 from SN38-TMS is slower at physiological pH (7.4): 24 h incubation released 26% SN38 while 48 h incubation released 63% SN38.

Tumor-Responsive Release of OxPt and SN38 from OxPt SN38:

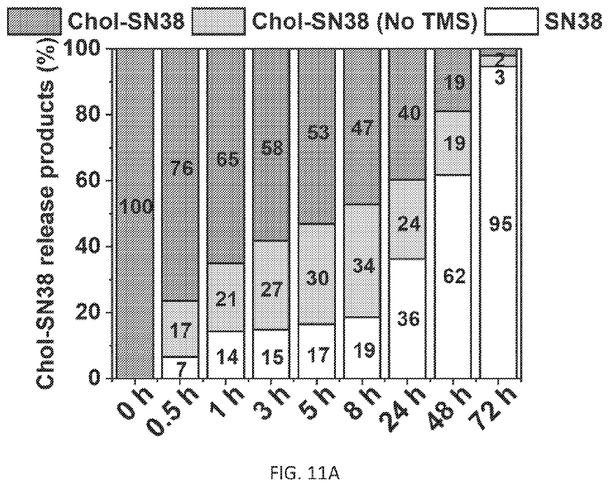

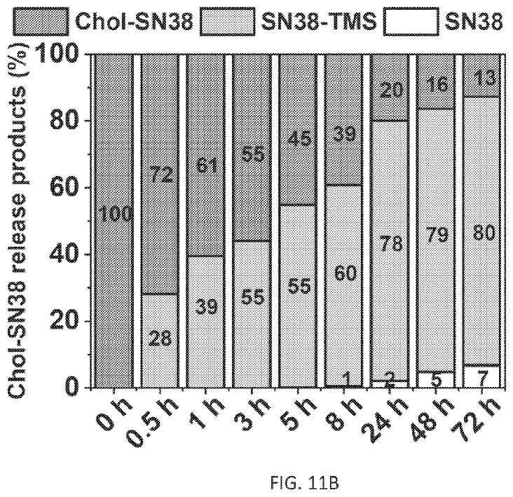

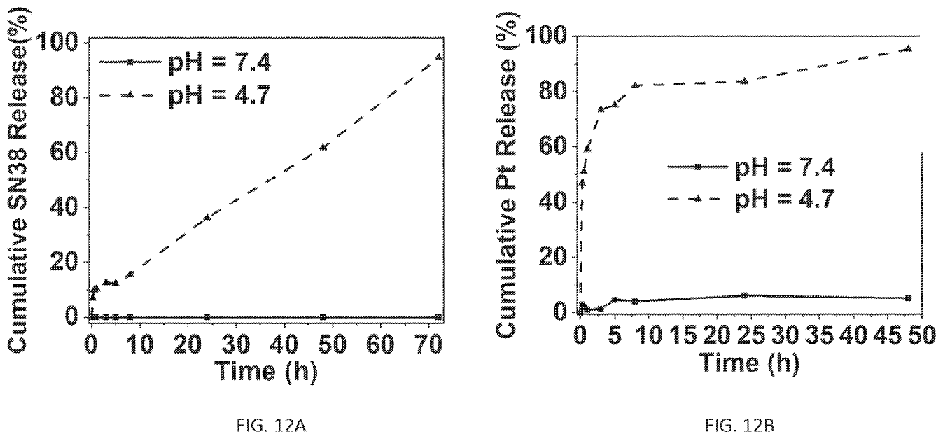

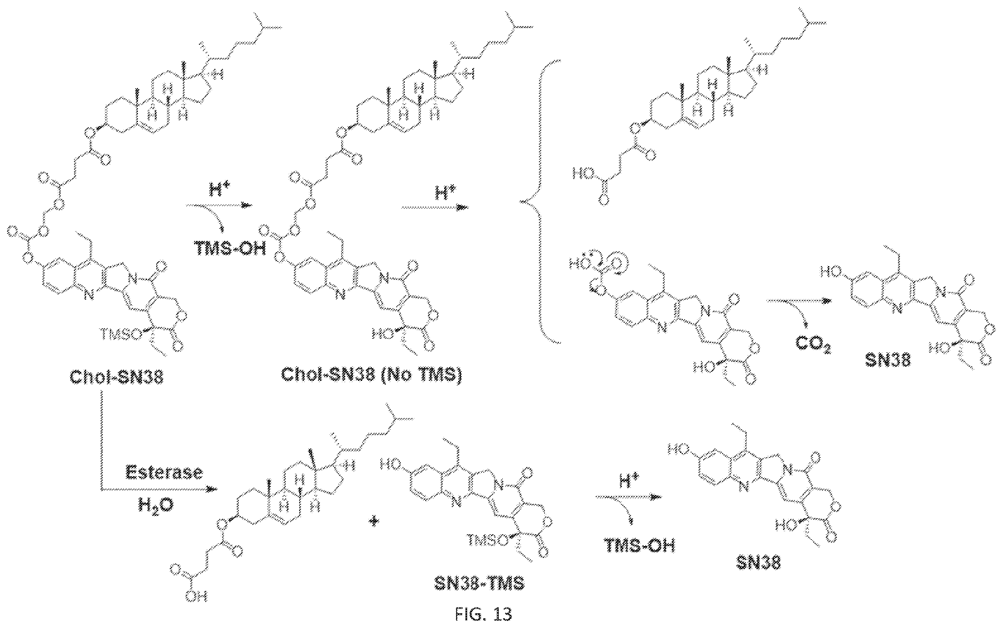

At pH=4.7, chol-SN38 from OxPt/SN38 was hydrolyzed at both the 20-OTMS and carbonate linkages to release SN38 in 95% yield in 72 h. See FIG. 11A. However, a negligible amount of SN38 was released from OxPt/SN38 at pH=7.4 in 72 h. See FIG. 111B. See also FIG. 12A. On the other hand, it is known that carboxyesterase in tumor cells contributes to the release of SN38 from irinotecan. The release of SN38 from OxPt/SN38 was tested in PBS with 10 unit/mL esterase. 28% Chol-SN38 was hydrolyzed to SN38-TMS in 0.5 h. After 72 h, only 13% Chol-SN38 remained while SN38-TMS and SN38 were generated in 80% and 7% yields, respectively. These results indicate the dual activation mechanisms for the release of SN38 from endocytosed OxPt/SN38 in endo/lysosomes: at low pH, 20-OTMS is quickly hydrolyzed to produce Chol-SN38 without TMS, which is further hydrolyzed in the carbonate linkage to afford SN38. See FIG. 13. In the meantime, the esterase in cancer cells hydrolyze the carbonate linkage to afford SN38-TMS, which is converted to SN38 via proton/TMS exchange. The tumor-responsiveness of chol-SN38 can potentially minimize the blood exposure of highly potent SN38, thus alleviating common adverse events associated with irinotecan treatment, such as neutropenia.

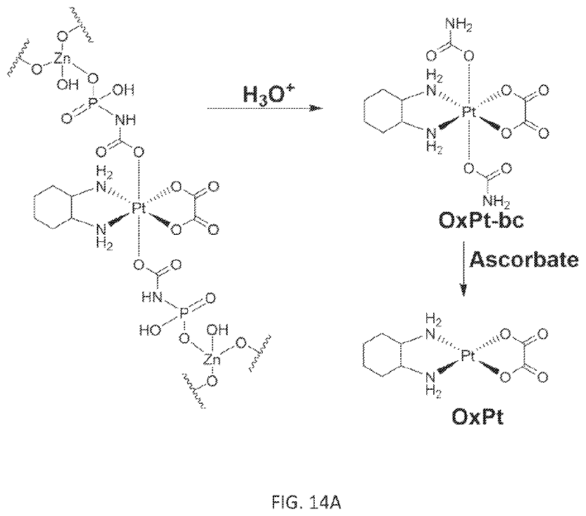

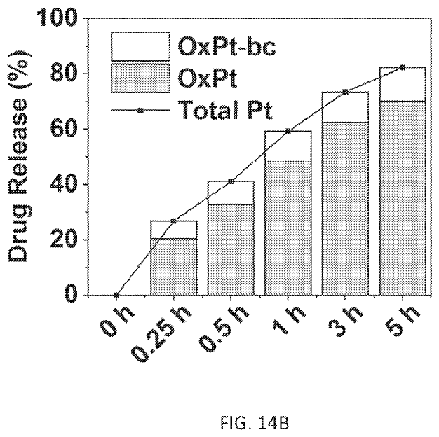

The NCP core formed by Zn²⁺ ions and OxPt-bp is known to disintegrate in acidic environments. See FIG. 14A. At pH=7.4 and 37° C., OxPt/SN38 released less than 6% Pt over a course of 48 h. At pH=4.7 and 37° C., OxPt/SN38 particles quickly disintegrated to release Pt(dach)(oxalate)(biscarbamate) (OxPt-bc) in 82% yield in 5 h and in 95% yield in 48 h. See FIG. 12B. In the presence of 5 mM ascorbate, the released OxPt-bc was efficiently reduced to form OxPt. In PBS with 5 mM ascorbate at pH=4.7, the OxPt-bp prodrug in OxPt/SN38 was reduced to afford OxPt in 70% yield in 5 h. See FIG. 14B. These results demonstrate triggered release of both SN38 and OxPt in cancer cells.

### Pharmacokinetics (PK) in Sprague Dawley Rats:

OxPt/SN38 at a dose of 2.0 mg OxPt/kg was intravenously (i.v.) injected to three Sprague Dawley rats via tail veins and 400 uL blood samples were collected at 5 min, 0.5 h, 1 h, 3 h, 5 h, 8 h, 24 h and 48 h from each rat. Blood samples was centrifuged at 10000 rpm for 10 min and plasma was collected for analysis. 200 μL metal-free concentrated nitric acid was added to 50 μL plasma and incubated for 48 h for complete digestion. Digested plasma was measured by ICP-MS for Pt concentration. See FIG. 2A. Another 50 μL plasma was diluted by 100 μL saturated NaCl solution and 100 μL 0.5% triton X-100 water solution, then extracted with 200 μL ethyl acetate. The organic layer was then analyzed using LC-HRMS for chol-SN38, SN38-TMS, and SN38 concentrations.

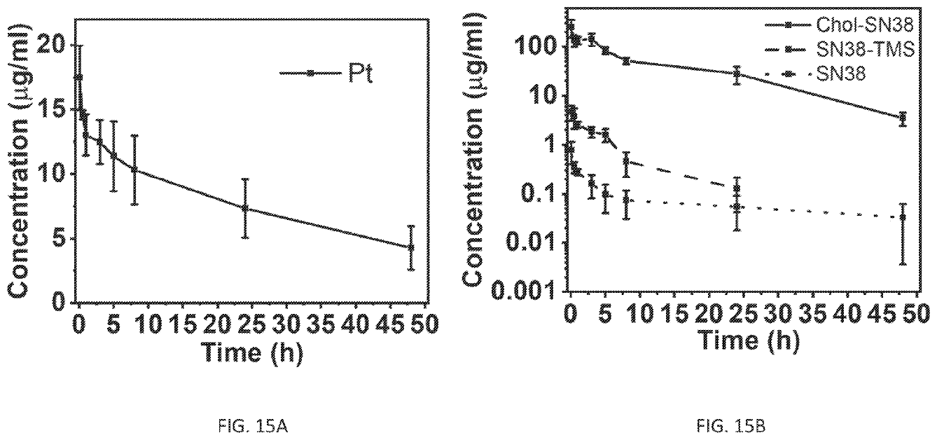

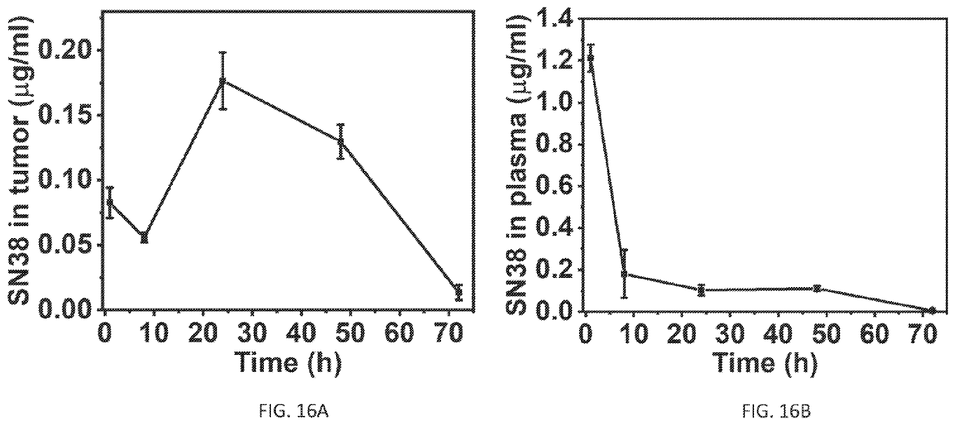

As shown in FIGS. 15A and 15B and in Table 4, below, OxPt in the particle core showed long circulation with a t_{1/2} of over 30 h and a Pt AUC_{0→∞} of 574 μg/ml*h, while chol-SN38 on the particle surface layer showed a t_{1/2} of 8 h and an AUC_{0→∞} of 1924 g/ml*h. TMS-SN38 and SN38 were detected in plasma with half-lives of 5.3 h and 2.2 h respectively, and AUC_{0→∞} values of 19 μg/ml*h and 4.4 μg/ml*h respectively. Thus, Chol-SN38 can slowly release TMS-SN38 and SN38 during circulation to maintain an effective concentration of SN38 for a prolonged period of time.

TABLE 4
Pharmacokinetics of OxPt/SN38 in Sprague Dawley rats.

| Model type | Parameters | Unit | Pt | Chol-SN38 | SN38-TMS | SN38 |
|---|---|---|---|---|---|---|
| Non-compartment | AUC_{0→inf} | (h · mg)/L | 574.5 ± 207.1 | 1924.4 ± 41.6 | 19.1 ± 3.6 | 4.4 ± 3.3 |
| model | AUMC_{0→inf} | (h · h · mg)/L | 25735.9 ± 13389.0 | 24547.3 ± 4376.1 | 100.5 ± 3.6 | 141.5 ± 15.6 |
| | CL | L/(h · kg) | 0.002 ± 0.001 | 0.009 ± 0.001 | 0.421 ± 0.087 | 2.053 ± 1.132 |
| | V_{SS} | L/kg | 0.077 ± 0.014 | 0.113 ± 0.024 | 2.049 ± 0.250 | 42.004 ± 7.592 |
| | MRT_{0→inf} | h | 42.99 ± 7.75 | 12.789 ± 2.505 | 5.072 ± 1.503 | 25.404 ± 13.660 |
| Two-compartment | A | mg/L | 5.89 ± 4.38 | 510.78 ± 270.10 | 2.23 ± 1.01 | 0.88 ± 0.27 |
| model | α | h⁻¹ | 1.69 ± 1.23 | 20.62 11.85 | 1.16 ± 0.11 | 2.53 ± 0.98 |
| | B | mg/L | 12.55 ± 2.89 | 157.79 ± 71.63 | 2.16 ± 0.39 | 0.18 ± 0.08 |
| | β | h⁻¹ | 0.024 ± 0.006 | 0.097 ± 0.040 | 0.130 ± 0.015 | 0.051 ± 0.003 |
| | t_{1/2α} | h | 1.045 ± 0.001 | 0.034 ± 0.003 | 0.599 ± 0.048 | 0.322 ± 0.124 |
| | t_{1/2β} | h | 30.23 ± 6.46 | 7.38 ± 1.77 | 5.34 ± 0.51 | 13.64 ± 0.81 |
| | k₁₀ | h⁻¹ | 0.037 ± 0.020 | 0.402 ± 0.205 | 0.236 ± 0.050 | 0.268 ± 0.026 |
| | k₁₂ | h⁻¹ | 0.511 ± 0.18 | 15.336 ± 8.853 | 0.415 ± 0.065 | 0.184 ± 0.783 |
| | k₂₁ | h⁻¹ | 1.165 ± 0.941 | 4.977 ± 2.848 | 0.637 ± 0.011 | 0.477 ± 0.169 |
| | AUC_{0→inf} | (h · mg)/L | 562.3 ± 202.7 | 1640.9 ± 282.2 | 18.6 ± 1.4 | 3.9 ± 3.3 |
| | AUMC_{0→inf} | (h · h · mg)/L | 25234.4 ± 12705.0 | 17030.9 ± 2603.1 | 130.0 ± 6.7 | 67.7 ± 15.6 |
| | MRT_{0→inf} | h | 43.01 ± 9.13 | 10.49 ± 2.57 | 6.99 ± 0.77 | 17.743 ± 1.132 |

### PK and Biodistribution (BD) in Tumor-Bearing Mice

1 million CT26 cells were inoculated on the right flank of 6-week old balb/c mice and OxPt/SN38 at a dose of 4 mg OxPt/kg was intravenously injected when the tumors reached larger than 200 mm³ in size (around 14 days), tumors of the mice were collected 1 h, 8 h, 24 h, 48 h and 72 h post injection. Blood samples were analyzed in the same way as described above for rat PK. Tumors were cut into pieces smaller than 1 mm³ and stirred to mix well, then about 20 mg tumor pieces were weighted and used for analysis. To determine OxPt distribution, 20 mg tumor sample was added into 200 μL metal-free concentrated nitric acid and incubated at 80° C. for 1 h and at room temperature for 72 h. Then 6.8 mL deionized water was added to each sample to dilute it into 2% nitric acid solution before ICP-MS analysis. To determine SN38, SN38-TMS, and chol-SN38 concentrations, each 20 mg sample was homogenized with 100 μL saturated NaCl solution and 100 μL 0.5% triton X-100 water solution, then extracted with 200 μL ethyl acetate. The ethyl acetate layer was collected for LC-HRMS analysis to determine SN38, SN38-TMS, and chol-SN38 concentrations.

Tumor had the highest Pt concentration of 9.7% ID/g at 24 h post injection. See FIGS. 16A and 16B. Chol-SN38 had the highest tumor accumulation of ˜5% ID/g at 24 h post injection. Chol-SN38 slowly released SN38 through the TMS-SN38 intermediate. SN38 concentration in tumor was maintained at 130 ng/ml at 48 h post injection, which is higher than IC₅₀ of SN38 (86 ng/ml). For tumor-bearing mice i.v. injected with OxPt/SN38 at a dose of 3 mg/kg OxPt, the AUC_{0→∞} of SN38 in plasma was around 12 μg/ml*h and in tumor reached 8.09±0.12 μg/ml*h. See Table 5, below.

TABLE 5
Pharmacokinetics of plasma and tumors in mice i.v.
injected with OxPt/SN38 at a dose of 3 mg OxPt/kg.

| Parameters | Unit | Plasma | Tumor |
|---|---|---|---|
| AUC_{0→inf} | (h · mg)/L | 12.59 ± 1.18 | 8.09 ± 0.12 |
| AUMC_{0→inf} | (h · h · mg)/L | 210.14 ± 11.30 | 276.08 ± 13.72 |
| CL | L/(h · kg) | 0.799 ± 0.072 | 1.236 ± 0.018 |
| V_{SS} | L/kg | 13.40 ± 1.79 | 42.18 ± 2.52 |
| MRT_{0→inf} | h | 16.73 ± 0.86 | 34.12 ± 1.82 |

### In Vivo Toxicity and Efficacy

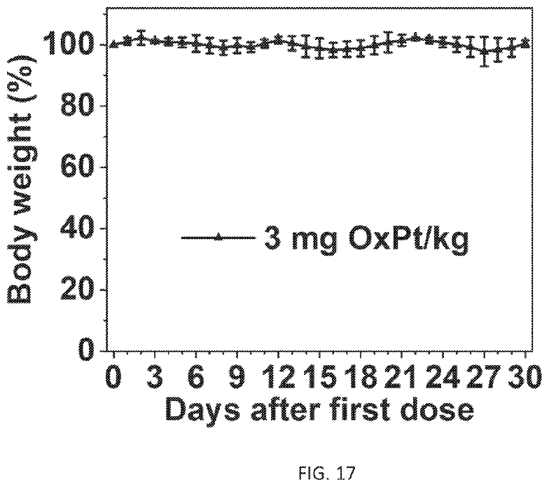

Rats and mice were i.v. injected with OxPt/SN38 at various dose levels and their body weight were monitored to determine drug tolerability. See FIG. 17.

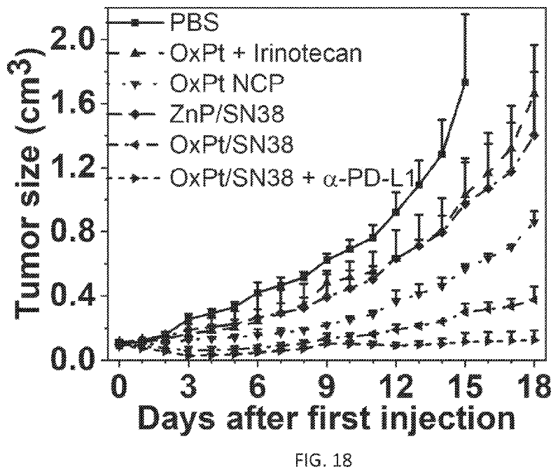

For mice dosed with OxPt/SN38 at 3 mg/kg OxPt once every 3 days (Q3D), there was no loss of body weight during repeated treatments, indicating this dose regimen is well tolerated. To determine the efficacy on colon cancer models, 1 million MC38 cells were inoculated on the right flanks of 6-week old C57bl/6 mice and the treatment started at day 7 when the sizes of tumors reached around 100 mm³. Mice were dosed with OxPt/SN38, OxPt NCP (particle with only OxPt prodrug in the core), ZnP/SN38 (particle with only Chol-SN38 on the shell), and OxPt plus irinotecan at an equivalent OxPt dose of 3 mg/kg on a Q3D schedule. 75 μg anti-PD-L1 antibody was also dosed intraperitoneally in one of the OxPt/SN38 groups. As shown in FIG. 18, both OxPt NCP and ZnP/SN38 delayed tumor growth. OxPt/SN38 showed markedly better antitumor efficacy. The addition of anti-PD-L1 antibody to OxPt/SN38 treatment further enhanced the antitumor efficacy to effectively control the tumors. This result indicates the synergy between immune checkpoint blockade and OxPt/SN38 treatment.

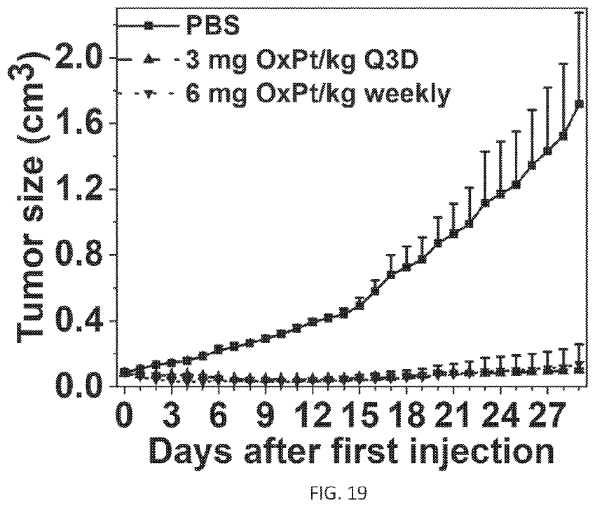

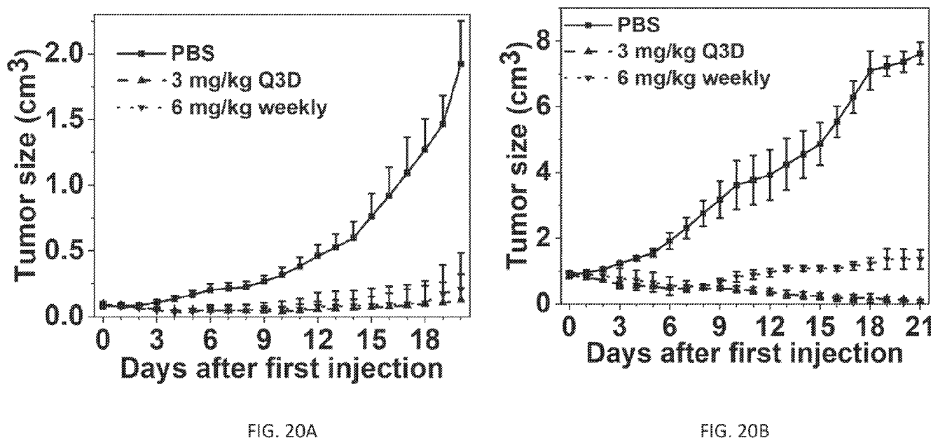

The anticancer efficacy of OxPt/SN38 on MC38 tumors were confirmed in another experiment with 3 mg/kg OxPt on a Q3D schedule and 6 mg/kg OxPt on a Q7D schedule. Both regimens effectively controlled the growth of MC38 tumors. See FIG. 19. Similar tumor growth inhibition was observed for CT26 tumors implanted in Balb/c mice. See FIG. 20A. Remarkably, OxPt/SN38 treatment of HT29 tumor bearing nude mice showed sustained tumor regression on a Q3D schedule at an OxPt dose of 3 mg/kg. See FIG. 20B. No significant weight loss was observed in all of these treated mice, suggesting that repeated doses of OxPt/SN38 with 3 mg/kg OxPt on a Q3D schedule or with 3 mg/kg OxPt on a Q7D schedule are well tolerated by mice.

Additional in vivo anticancer studies on of OxPt/SN38 were conducted on subcutaneous MC38 and CT26 murine and HT29, HCT116 and SW480 human colorectal adenocarcinoma models. When tumors reached 80-120 mm³ in volume, mice were injected intravenously different treatments every three days (Q3D). OxPt/SN38, OxPt plus irinotecan, OxPt NCP, and ZnP/SN38 were dosed at 3.5 mg OxPt/kg equivalent and 15.9 mg Chol-SN38/kg (equivalent to 6.2 mg/kg SN38), 3.5 mg OxPt/kg and 20.2 mg/kg irinotecan (11.7 mg/kg SN38 equivalent), 3.5 mg OxPt/kg equivalent, and 15.9 mg Chol-SN38/kg (equivalent to 6.2 mg/kg SN38), respectively. In all tested models, OxPt/SN38 led to significantly better tumor growth inhibition/regression with minimal toxicity judged by both body weight, histology of major organs, and liver and renal function tests.

MC38 tumor-bearing C57BL/6 mice following 8 intravenous injections of OxPt/SN38 showed 92.2% tumor growth inhibition (TGI, defined as 1−(RTVt/RTVc) where RTV=endpoint tumor volume). Despite a 1.9-fold higher SN38 equivalent dose, OxPt plus irinotecan provided a modest TGI of 22.3%. OxPt NCP and ZnP/SN38 modestly inhibited tumor growth with TGI values of 66.9% and 16.4%, respectively. The mice tolerated the treatments well with steady body weights for all groups.

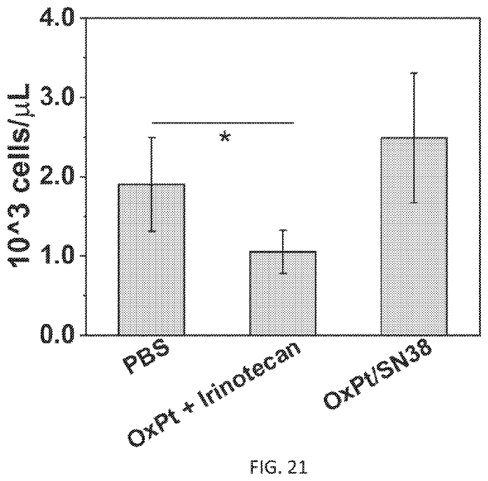

Neutropenia is typically the most serious side effect for the IROX regimen, with 30% mCRC patients experiencing severe neutropenia after repeated doses of OxPt plus irinotecan. Blood samples were collected from MC38 tumor-bearing C57BL/6 mice following 8 intravenous injections of PBS or OxPt/SN38 or 3 intravenous injections of OxPt plus irinotecan. Flow cytometric analysis showed that the absolute neutrophil count (ANC) decreased in mice treated with OxPt plus irinotecan compared to PBS control, but slightly increased for mice treated with OxPt/SN38 compared to PBS control. See FIG. 21. OxPt/SN38 treatment thus overcame the dose-limiting toxicity of severe neutropenia in the IROX regimen.

Liver and kidney functions were determined by measuring the levels of alanine aminotransferase (ALT), aspartate aminotransferase (AST), and serum creatinine levels in the sera of C57BL/6 mice after 8 and 15 doses of PBS or OxPt/SN38. The AST and ALT levels for mice treated with OxPt/SN38 slightly increased over the PBS control, but are well within the normal ranges. The creatinine level remained unchanged between mice treated with OxPt/SN38 (0.24±0.07 mg/dL) and those treated with PBS (0.22±0.01 mg/dL). These results indicate that repeated doses of OxPt/SN38 do not cause hepatotoxicity and nephrotoxicity in mice.

In a mouse colorectal cancinoma (CT26) model, OxPt/SN38, OxPt plus irinotecan, OxPt NCP, and ZnP/SN38 showed TGI values of 90.9±7.1%, 27.6±9.9%, 51.6±18.7%, and 29.1±19.7%, respectively. OxPt/SN38 thus showed strong synergy between the two drugs to afford much enhanced anticancer efficacy over other groups. All mice tolerated the treatments well with no obvious decrease of bodyweights.

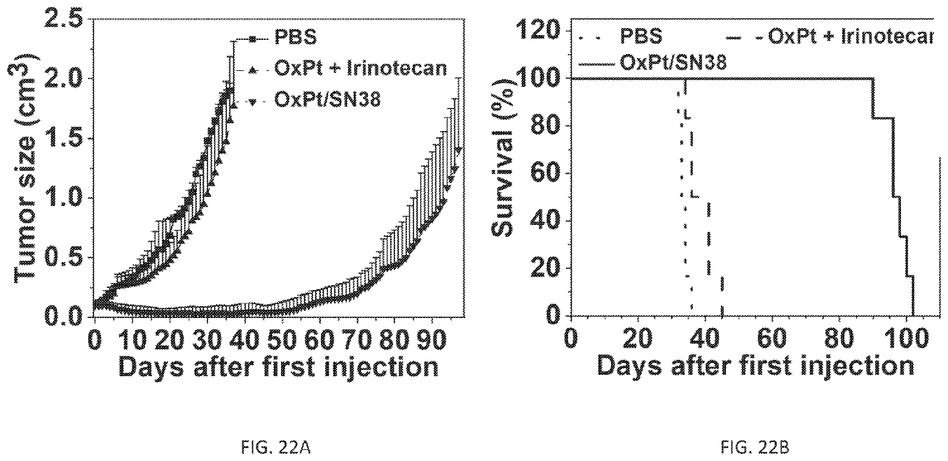

OxPt/SN38 also showed anticancer efficacy on mouse xenograft models of human colorectal adenocarcinoma. The tumor-bearing mice were intravenously injected with PBS, OxPt/SN38, or OxPt plus irinotecan for 16 doses. For HT29 model, OxPt/SN38 regressed the tumors to afford a TGI of 99.4±0.4% at the endpoint of PBS group. See FIG. 22A. OxPt plus irinotecan only slightly inhibited the tumors to afford a TGI of 32.2±22.5%. After the cessation of OxPt/SN38 treatment on Day 45, HT29 tumors were inhibited for 12 more days but eventually regrew after Day 57. OxPt/SN38 and OxPt plus irinotecan treatments extended the median survival from 33 days for PBS control to 97 and 36 days, respectively. See FIG. 22B. ZnP/SN38 at a dose of 36 mg Chol-SN38 also effectively inhibited HT29 tumor growth with a TGI of 87.0±3.9%. The mice in all groups tolerated the treatments well.

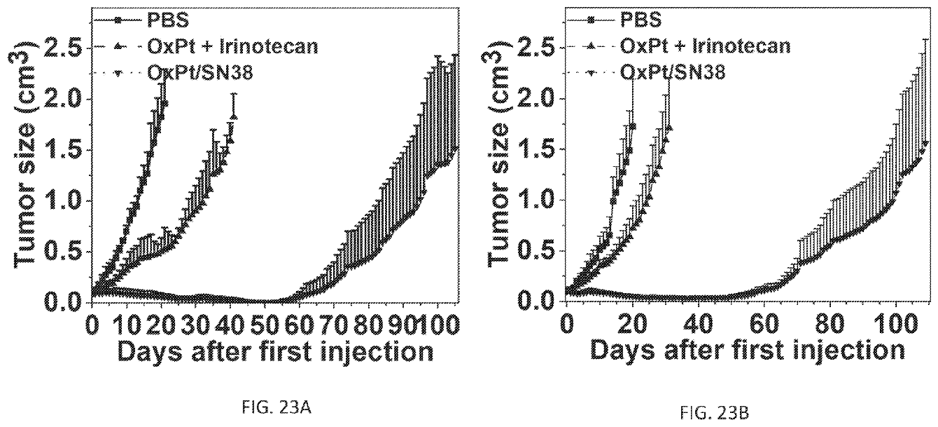

OxPt/SN38 also showed antitumor efficacy on HCT116 and SW480 tumor models. For the HCT116 model, OxPt/SN38 regressed tumors to afford a TGI of 98.7±0.8% at the endpoint of PBS group on Day 18. OxPt plus irinotecan showed a TGI of 72.4±9.7% on Day 18. See FIG. 23A. OxPt/SN38 and OxPt plus irinotecan extended mouse survival from 18 days for PBS group to 106 and 40 days, respectively. For the SW480 model, OxPt/SN38 regressed tumors to afford a TGI of 96.9±1.0% at the endpoint of PBS group on Day 17 while OxPt plus irinotecan gave a moderate TGI of 57.9±12.8%. See FIG. 23B. OxPt/SN38 and OxPt plus irinotecan extended mouse survival from 17 days for PBS group to 109 and 32 days, respectively. Thus, OxPt/SN38 has a unique mode of action, excellent antitumor efficacy on five CRC tumor models, and good safety profiles.

### LDLR-Mediated Endocytosis Determines the Anticancer Efficacy of OxPt SN38:

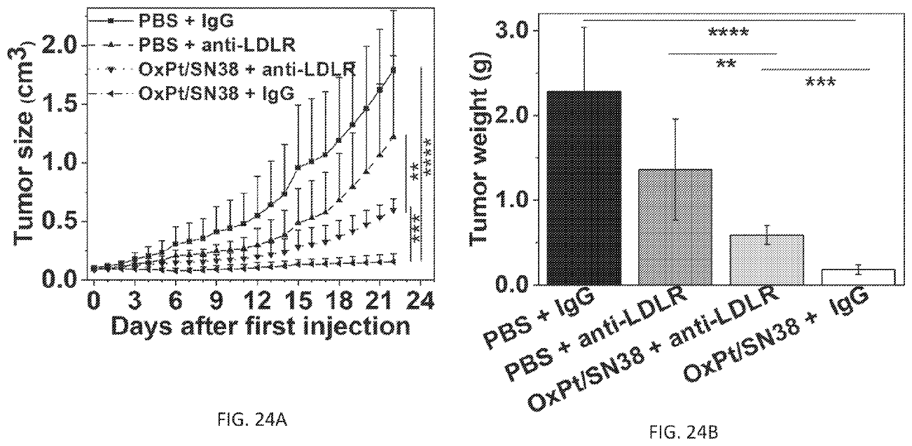

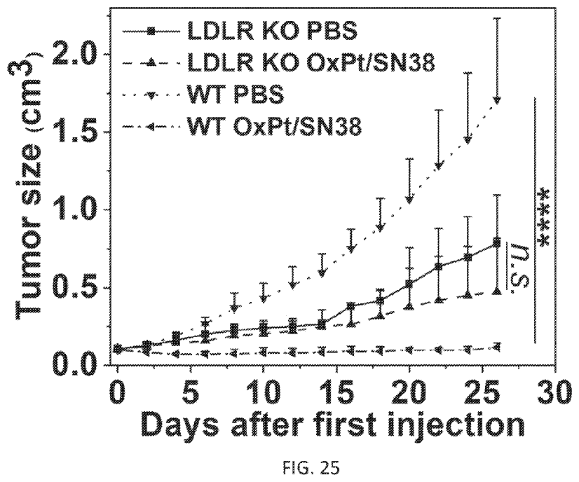

Tumor growth inhibition of intravenously injected OxPt/SN38 with concurrent LDLR blocking by intratumorally injecting 1 μg of a-LDLR on a Q3D schedule was studied to determine the role of LDLR-mediated endocytosis on anticancer efficacy. See FIG. 24A. While a-LDLR slowed tumor growth with a TGI of 40.2±19.4% over the IgG control, OxPt/SN38 treatment with concurrent LDLR blocking significantly weakened the antitumor effect with a TGI of 51.6±5.5% compared to a TGI of 91.2±3.8% for OxPt/SN38 treatment with concurrent IgG injection. The results were corroborated by the tumor weights at the endpoint: OxPt/SN38 treatment with concurrent IgG injection showed a TGI of 92% while OxPt/SN38 treatment with concurrent LDLR blocking showed a TGI of 57%. See FIG. 24B. These LDLR blocking results were confirmed by establishing subcutaneous tumors using LDLR knockout MC38 tumor cells. The anticancer efficacy of OxPt/SN38 was nearly abrogated in LDLR knockout tumors with no significant difference between OxPt/SN38 and PBS groups. See FIG. 25.

In vivo cytotoxicity on tumor cells was further examined through histopathological analysis. H&E staining showed severe necrosis in MC38 tumors treated with OxPt/SN38 but much less necrosis in MC38 tumors treated with OxPt/SN38 and a-LDLR. TUNEL and Caspase 3 IHC staining showed strong apoptosis induced by OxPt/SN38, but greatly reduced apoptosis when LDLR was blocked. These results show that LDLR-mediated endocytosis is involved in the tumor uptake of OxPt/SN38 in vivo and plays a role on antitumor efficacy.

### Discussion:

For decades, organic cytotoxic anticancer drugs such as SN38 (Log P=3.37) have been modified with hydrophilic groups to render them soluble or slightly soluble in aqueous solution. Conversion of water-insoluble SN38 into water-soluble irinotecan hydrochloride (Log P=−0.45) represents one of the most successful examples of organic anticancer drug designs. Binding of hydrophobic chemotherapeutics to plasma proteins presents an alternative approach and can actively target highly expressed receptors. For example, nab-paclitaxel (albumin-paclitaxel nanoparticles) showed better efficacy than paclitaxel in some tumors, presumably via targeting the Gp60 transcytosis pathway in endothelial cells and binding to secreted protein, acidic and rich in cysteine (SPARC) in the tumor extracellular matrix. According to one aspect of the presently disclosed subject matter, a new strategy to co-deliver combination chemotherapies via active targeting of LDLR in tumors is disclosed. Here SN38 was conjugated to highly hydrophobic cholesterol (Log P=7.02) through a labile acetal linkage to hijack LDL, a lipoprotein responsible for the transfer of cholesterol and related derivatives that are essential for rapidly growing tumor cells. The acetal linker in Chol-SN38 was selectively cleaved in tumors to release SN38 via both acid- and esterase-catalyzed hydrolysis. The presently disclosed strategy makes it possible to design hydrophobic prodrugs for tumor targeting via LDRD-mediated endocytosis.

Systemically injected nanotherapeutics have long been shown to prolong blood circulation over their parent drugs. It was previously believed that long-circulating nanoparticles preferentially accumulated in tumors as a result of the EPR effect. Herein it is shown that rationally designed core-shell NCP particles not only provided for the loading of both hydrophilic OxPt-bp and hydrophobic Chol-SN38 prodrugs but also actively targeted LDLR to significantly enhance drug uptake in tumors. Chol-SN38 strongly bonded to LDL, leading to efficient transfer of Chol-SN38 from the shell of OxPt/SN38 to LDL for active transport to tumors via LDLR-mediated endocytosis. SN38 was selectively released inside tumor cells via acid- and esterase-catalyzed hydrolysis. On the other hand, the NCP core of OxPt/SN38 adsorbed Apo B-100 in plasma to allow tumor targeting via the LDLR pathway and preferentially released OxPt in tumors via acid-triggered disintegration in the endo/lysosomes and reduction by ascorbate and other intracellular reductants. As a result, OxPt/SN38 significantly increased tumor deposition of OxPt by a factor of 4.9 over OxPt and SN38 by a factor of 6 over irinotecan at equivalent doses.

IROX is one of the standard chemotherapy regimens for mCRC due to the synergistic actions of OxPt and SN38 on CRC cells. By increasing intratumoral OxPt and SN38 concentrations, OxPt/SN38 maximized the synergy between OxPt and SN38 in vitro and in vivo on murine and human CRC cells. OxPt/SN38 simultaneously crosslinked DNA with OxPt and inhibited topoisomerase 1 with SN38, resulting in severe DNA damage, inhibition of DNA replication, and disruption of mitochondrial membranes. OxPt/SN38 achieved >92% tumor growth inhibition on MC38 and CT26 murine CRC tumor models and >97% tumor growth inhibition on HT29, HCT116, and SW480 CRC tumor models. OxPt/SN38 also prolonged mouse survival by 64, 88, and 92 days compared to PBS control and by 61, 66, and 77 days compared to OxPt plus irinotecan on HT29, HCT116, and SW480 CRC tumor models, respectively. OxPt/SN38 achieved excellent antitumor efficacy in multiple mouse models of CRC without causing serious side effects such as neutropenia and impairment of liver and kidney functions.

### Example 2

### Nanoscale Coordination Polymer Core-Shell Nanoparticles Codeliver Oxaliplatin and Paclitaxel

### Synthesis of Cholesterol-Paclitaxel (Chol-PTX)

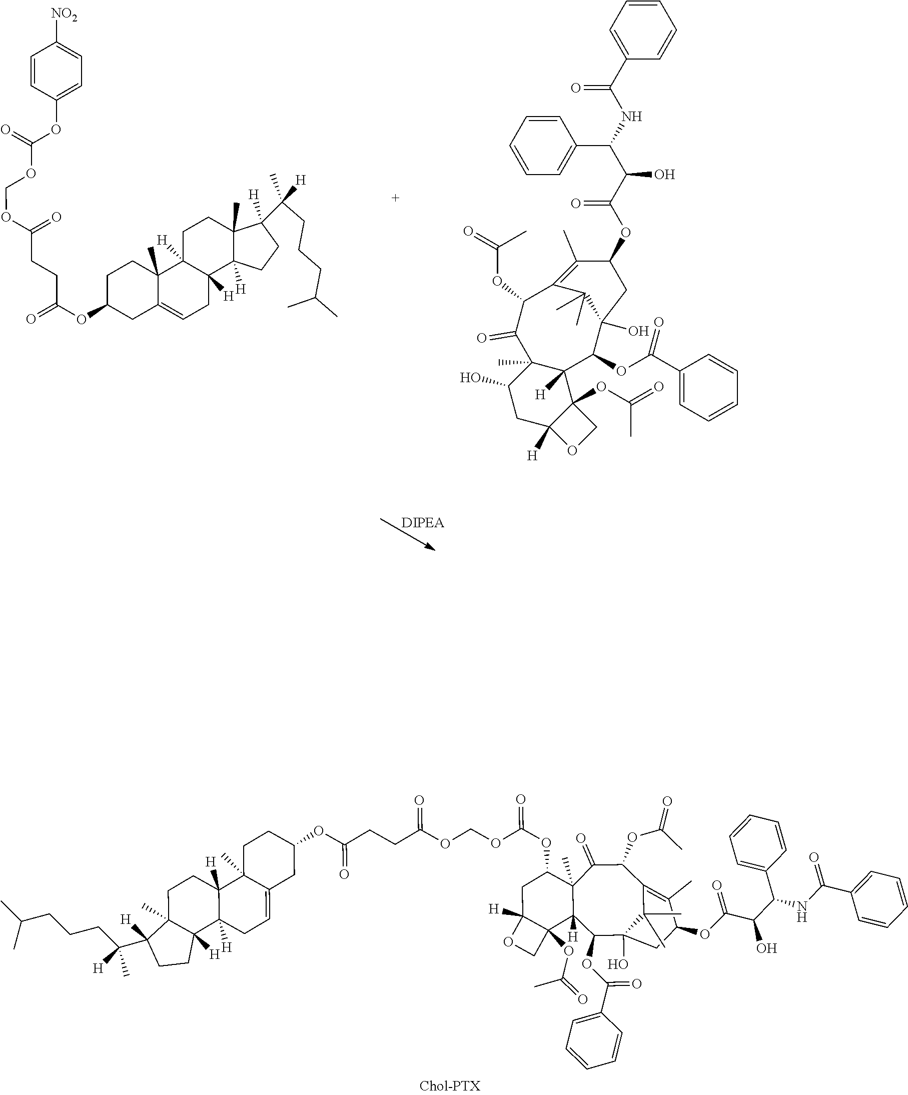

1 g (1.5 mmol) cholesterol linker and paclitaxel (1.3 g, 1.5 mmol, 1 eq) were dissolved in 50 mL anhydrous DCM. 1 mL (6 mmol, 4 eq) DIPEA was added to the solution and the solution was stirred at room temperature under nitrogen for 24 h. The resulting deep yellow solution was diluted with 100 mL DCM and washed with saturated NaHCO₃ three times, followed by 1M HCl and saturated NaCl. The organic layer was then dried over anhydrous Na₂SO₄ for 2 h and concentrated on a rotary evaporator. The product was further purified by column chromatography using silica gel. The product was eluted with 1.5:1 Hexanes:EtOAc. Yield: 0.36 g (0.26 mmol, 17%). ¹H NMR (500 MHz, Chloroform-d) δ 8.13 (d, 2H), 7.75 (d, 2H), 7.53 (m, 1H), 7.37-7.43 (m, 10H), 7.08 (d, 1H), 6.29 (s, 2H), 6.02 (d, 1H), 5.78 (dd, 2H), 5.73 (d, 1H) 5.47 (d, 1H), 5.38 (d, 1H), 4.95 (d, 1H), 4.62 (m, 1H), 4.45 (m, 1H), 4.28 (dd, 2H), 3.85 (d, 1H), 2.10-2.90 (m, 16H), 0.80-2.10 (m, 52H), 0.67 (s, 3H). ESI-MS: m/z=1413.7263 (expected 1413.7255 for [M+NH₄]⁺).

The IC₅₀ values for Chol-PTX was determined to be 3.95 μM, in comparison to an IC₅₀ of 0.24 μM for LL/2 lung cancer cells.

### Synthesis and Characterization of OxPt PTX Core-Shell Nanoparticle

0.21 mg cholesterol, 0.42 mg DOPC, 0.75 mg DSPE-PEG2k, 0.25 mg chol-PTX and 0.5 mg OxPt bare NCP particle was mixed in 80 uL THF and added to 500 μL 30% EtOH with 1700 rpm stirring at 50° C. The mixture was concentrated to 100 uL to obtain OxPt/PTX core-shell nanoparticle. Z-average diameter and PDI of the OxPt/SN38 particle were 101.1 nm and 0.093 by DLS.

### Example 3

### Nanoparticles Deliver Podophyllotoxin

### Synthesis of Cholesterol-Podophyllotoxin (Chol-PPX)

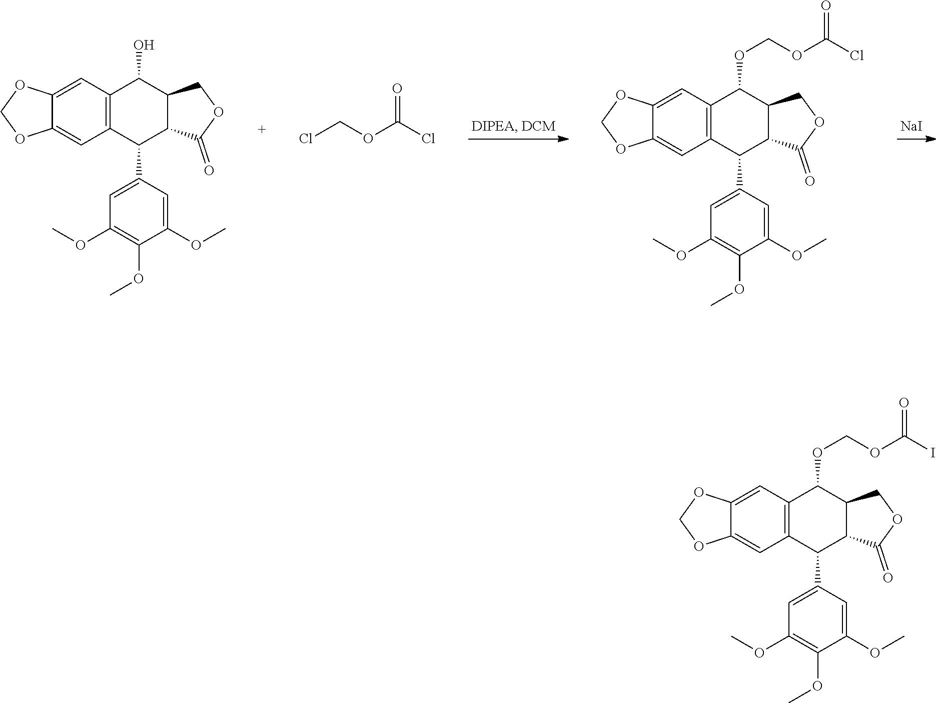 [SMILES: COc1cc([C@@H]2c3cc4c(cc3[C@H](O)[C@H]3COC(=O)[C@H]23)OCO4)cc(OC)c1OC ; O=C(Cl)OCCl ; COc1cc([C@@H]2c3cc4c(cc3[C@H](OCOC(=O)Cl)[C@H]3COC(=O)[C@H]23)OCO4)cc(OC)c1OC ; "NaI" ; COc1cc([C@@H]2c3cc4c(cc3[C@H](OCOC(=O)I)[C@H]3COC(=O)[C@H]23)OCO4)cc(OC)c1OC]

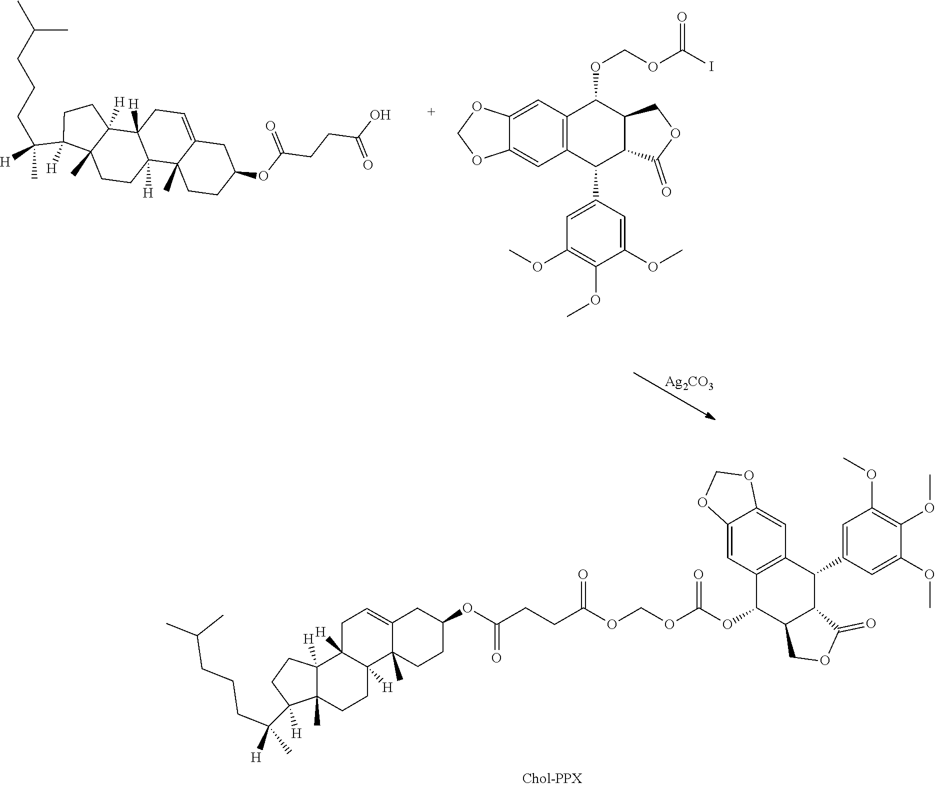

PPX (0.41 g) and DIPEA (0.13 g) were suspended in dry DCM (100 mL). Chloromethyl chloroformate (0.13 g) was added to the PPX suspension in an ice-water bath and the resulting mixture was stirred at room temperature for 24 h. The solution was washed with water (3×20 mL) and brine (3×20 mL), and then dried with anhydrous Na₂SO₄. The organic solvent was evaporated under reduced pressure and the resulting PPX-Cl was dried under vacuum overnight (Yield: 0.33 g, 65%).

PPX-Cl (0.51 g) was dissolved in dry acetone (75 mL). NaI (0.15 g) was then added. The mixture was stirred at 50° C. in the dark for 24 h. The organic solvent was removed, and the product was re-dissolved in ether (150.0 mL). The solution was washed with 10% Na₂SO₃ (3×20 mL), water (3×20 mL), and brine (3×20 mL), then dried with anhydrous Na₂SO₄. The organic solvent was evaporated under reduced pressure and the resulting PPX-I was dried under vacuum overnight (Yield: 0.50 g, 83%).

Cholesterol-COOH (0.49 g) was dissolved in acetonitrile (20 mL). PPX-I (0.60 g) dissolved in acetonitrile (50 mL) was added dropwise to the cholesterol-COOH solution in an ice-water bath, followed by the addition of excess silver carbonate (2.76 g). The mixture was stirred at 80° C. for 36 h. After filtration to remove the precipitate, the organic solvent was removed. The residue was re-dissolved in ethyl acetate (100.0 mL) and the solution was washed with 10% NaHCO₃ (3×20 mL), water (3×20 mL), and brine (3×20 mL), then dried with anhydrous Na₂SO₄. The organic solvent was evaporated under reduced pressure and the crude product was purified by silica gel flash column chromatography using DCM:ethyl acetate (3:1) as eluent to afford chol-PPX (Yield: 0.29 g, 30%). ¹H NMR (500 MHz, CDCl3): 0.66-2.05 (m, 41H, cholesterol), 2.30 (d, 2H, CH₂), 2.64 (t, 2H, CH₂), 2.72 (t, 2H, CH₂), 2.94 (m, 2H, CH, CH), 3.74 (s, 6H, CH₃, CH₃), 3.80 (s, 3H, CH₃), 4.23 (t, 1H, CHH), 4.49 (t, 1H, CH), 4.60 (m, 2H, CH, CHH), 5.34 (m, 1H, OCH), 5.82 (m, 3H, CH, OCH₂O), 5.99 (d, 2H, OCH₂O), 6.37 (s, 2H, CH, CH), 6.54 (s, 1H, CH), 6.87 (t, 1H, CH). The m/z of [M+NH₄]⁺ was determined to be 974.5275 (expected m/z=974.5266).

### Synthesis of PPX-NP

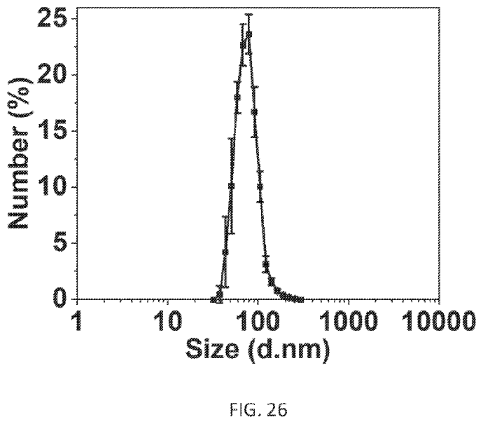

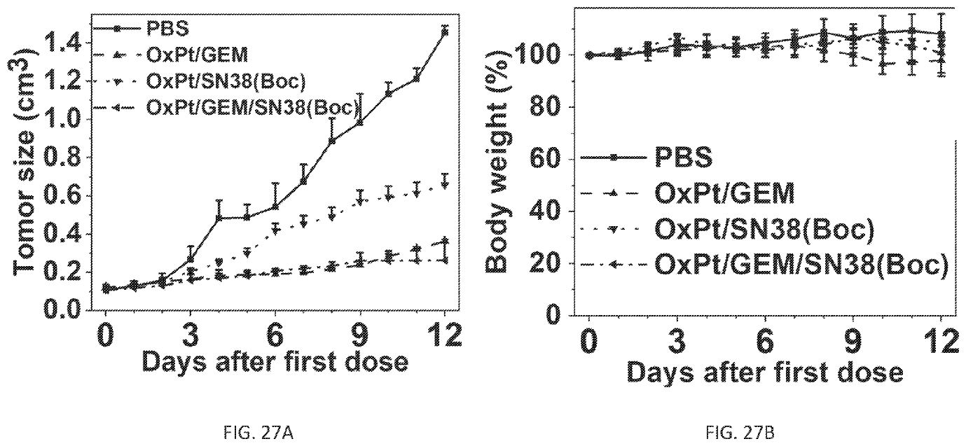

0.21 mg cholesterol, 0.42 mg DOPC, 0.75 mg DSPE-PEG2k, 0.2 mg chol-PPX and 0.5 mg Carboplatin bare NCP particle was mixed in 80 uL THF and added to 500 uL 30% EtOH with 1700 rpm stirring at 50° C. The mixture was concentrated to 100 uL to obtain Carbo/PPX core-shell nanoparticle. Z-average diameter and polydispersity (PDI) of the Carbo/PPX particle were 98.9 nm and 0.111 by dynamic light scattering (DLS). See FIG. 26.

### Example 4

### Nanoparticles Codeliver Oxaliplatin, Gemcitabine and SN38

### Synthesis of Nanoparticles with Various Chol-SS-SN38 (the Disulfide Linker)

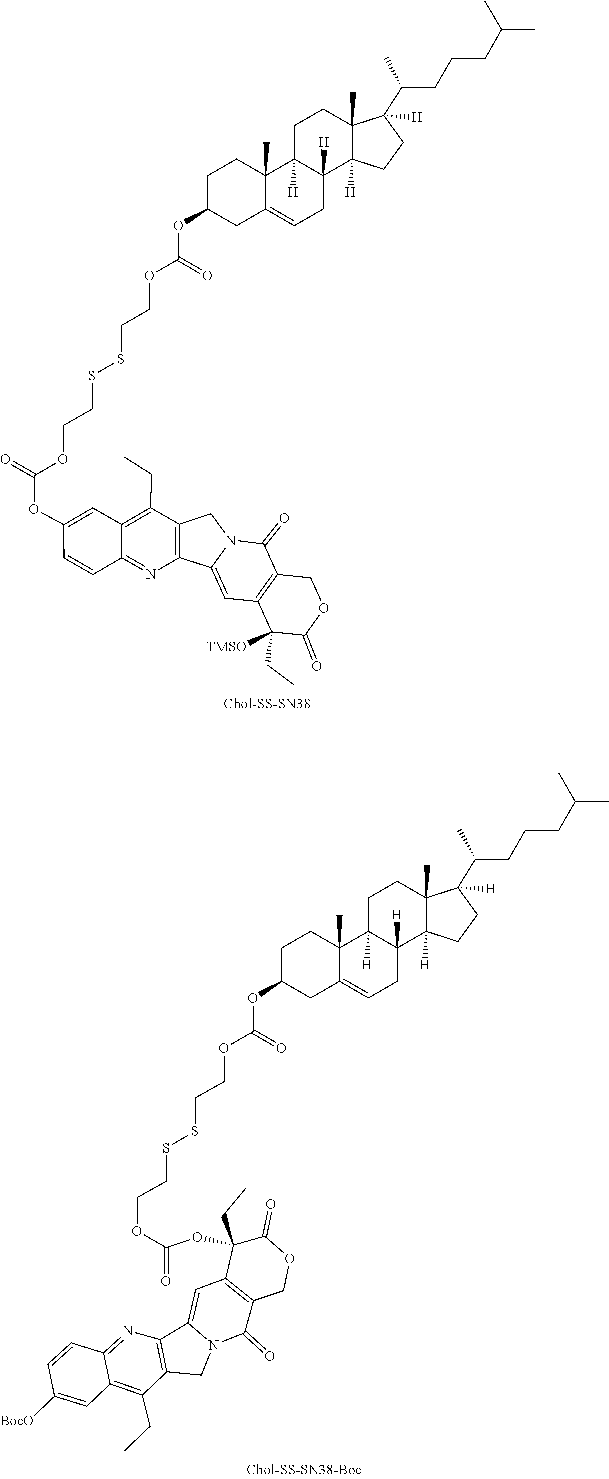

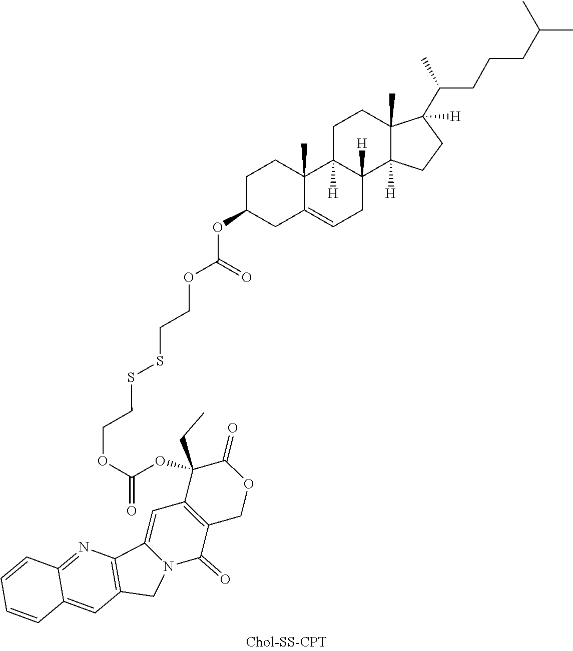

0.21 mg cholesterol, 0.42 mg DOPC, 0.75 mg DSPE-PEG2k, 0.2 mg Chol-SS-SN38 and 0.5 mg OxPt/gemcitabine(GEM) bare NCP particle (C. Poon et al. Journal of Controlled Release 201 (2015) 90-99) were mixed in 80 μL THF and added to 500 uL 30% EtOH with 1700 rpm stirring at 50° C. The mixture was concentrated to 100 μL to obtain OxPt/GEM/Chol-SS-SN38 core-shell nanoparticle. Z-average diameter and PDI of the OxPt/GEM/Chol-SS-SN38 particle (also referred to herein as “OxPt/GEM/SN38-Boc”, synthesized with Chol-SS-SN38-Boc were 96.79 nm and 0.211 by DLS. The nanoparticles made with Chol-SS-SN38 and Chol-SS-CPT show larger sizes. See Table 6, below.

TABLE 6
DLS data of nanoparticles made by
various Chol-disulfide Prodrugs

| | Z-Average (nm) | PDI |
|---|---|---|
| Chol-SS-SN38 | 172.3 | 0.38 |
| Chol-SS-SN38-Boc | 96.79 | 0.211 |
| Chol-SS-CPT | 263.2 | 0.434 |

### In Vivo Efficacy of OxPt/GEM Chol-SS-SN38-Boc:

To determine the efficacy on mouse colon cancer model, 1 million CT26 cells were inoculated on the right flanks of 6-week old Balb/c mice and the treatment started at day 7 when the sizes of tumors reached around 100 mm³. Mice were dosed with OxPt/GEM/Chol-SS-SN38-Boc) with a molar ratio of 4:1:1 at a dose of 3 mg/kg OxPt on a Q3D schedule (FIG. 10). OxPt/GEM and OxPt/Chol-SS-SN38-Boc with equal active agent amounts served as controls. In the two drug combination groups, both OxPt/GEM and OxPt/Chol-SS-SN38-Boc showed obvious tumor growth delay. See FIG. 27A. For three drug combination, OxPt/GEM/Chol-SS-SN38-Boc exhibited a tumor growth inhibition of 86.7%. No significant weight loss was observed in the various mouse treatment groups. See FIG. 27B.

### Example 5

### NCP Core-Shell Particles Codeliver Oxaliplatin, Gemcitabine Monophosphate, and SN38

Synthesis and characterization of OxPt/GEM/SN38 core-shell nanoparticle NCP particles with OxPt-bp and gemcitabine monophosphate (GMP) in the core and Chol-SN38 on the shell, referred to as “OxPt/GMP/SN38,” was synthesized in two steps. OxPt/GMP-bare particles were synthesized according to our previously reported method with minor modifications (Duan, X.; et al. Nat. Commun. 2020, 10: 1899). Briefly, an aqueous solution of OxPt-bp (30 mg, 150 mg/mL) and GMP (8 mg, 40 mg/mL) was added to a 5 mL of 0.3M Triton X-100/1.5M 1-hexanol in cyclohexane and stirred vigorously for 15 min in the presence of DOPA (30 mg, 200 mg/mL in CHCl₃). An aqueous solution of Zn(NO₃)₂ (60 mg, 600 mg/mL) was added to a 5 mL of 0.3M Triton X-100/1.5M 1-hexanol in cyclohexane and stirred vigorously for 5 min. The Zn(NO₃)₂-containing microemulsion was added dropwise to the OxPt-bp-containing microemulsion and stirred vigorously for 30 min at room temperature. After the addition of 10 mL ethanol, OxPt-bare was obtained by centrifugation at 11,628×g. The resulting pellet was washed twice with THF/ethanol and finally re-dispersed in THF. The loadings of OxPt in the particles were determined by ICP-MS (Agilent 7700x, Agilent Technologies, Santa Clara, California, United States of America) after digestion with nitric acid. The particle size and zeta potential were determined by dynamic light scattering using a Zetasizer (Nano ZS, Malvern, United Kingdom). Z-averaged diameter and PDI of the OxPt/SN38-bare particle were 55.4 nm and 0.147 by DLS.

OxPt/GMP/SN38 was prepared by adding a THF solution (80 μL) of DOPC, cholesterol, DSPE-PEG₂ₖ, Chol-SN38, and OxPt/GEM to 500 μL of 30% (v/v) ethanol/water at room temperature. The DOPC:Chol:DSPE-PEG:Chol-SN38 molar ratio was 3:3:1.5:1. The mixture was stirred at 1700 rpm for 1 min. THF and ethanol were completely evaporated and the solution was allowed to cool to room temperature. Z-averaged diameter and PDI of the OxPt/GMP/SN38 particle were 77.94 nm and 0.145 by DLS.

### In Vitro Cytotoxicity of OxPt GMP SN38 in Pancreatic Cell Lines:

Murine pancreatic KPC and Panc02 cells were seeded into 96-well plates at 2500 cells/well for 24 h. Oxaliplatin, SN38, Chol-SN38, Gemcitabine (GEM), GMP, OxPt NCP (single drug), ZnP/SN38 (single drug), GMP NCP (single drug), OxPt/SN38 (double drugs) and OxPt/GMP/SN38 (three drugs) at various concentrations were dosed to each well and the cells were incubated for another 48 h. The cell viability was measured by MTS assay.

As listed in Tables 7 and 8, below, OxPt, SN38 and GEM showed high cytotoxicity on KPC and Panc02 cells. As a key metabolite to the active gemcitabine triphosphate, GMP showed the lowest IC₅₀ of 0.30±0.07 μM and 0.06±0.02 μM for KPC and Panc02, respectively. Although the modification of SN38 into Chol-SN38 and OxPt into OxPt NCP slightly lowered their cytotoxicity, the combination of two or three drugs showed strong synergy to reduce their IC₅₀ values by 1.2-1.8 folds or 8.5-20 folds, respectively.

TABLE 7
OxPt, GMP, and SN38 IC₅₀ values (μM) in KPC cells^{a}

| OxPt | SN38 | Chol-SN38 | GEM | GMP |
|---|---|---|---|---|
| 5.94 ± 1.73 | 0.30 ± 0.09 | 3.18 ± 1.01 | 0.67 ± 0.18 | 0.30 ± 0.07 |

| OxPt | | | | OxPt/GEM/ |
|---|---|---|---|---|
| NCP | ZnP/SN38 | GMP NCP | OxPt/SN38^{b} | SN38^{b} |
| >200 | 10.46 ± 4.03 | 0.94 ± 0.28 | 4.23 ± 1.30 | 0.22 ± 0.03 |

^{a}Molar ratio for OxPt/SN38 had an OxPt:SN38 molar ratio of 1:2 and OxPt/GMP/SN38 had an OxPt:GMP:SN38 molar ratio of 2:1:4.
^{b}The IC50 values were calculated based on OxPt.

TABLE 8
OxPt, GMP, and SN38 IC₅₀ values (μM) in Panc02 cells^{a}

| OxPt | SN38 | Chol-SN38 | GEM | GMP |
|---|---|---|---|---|
| 14.04 ± 4.88 | 0.17 ± 0.12 | 14.63 ± 4.87 | 0.11 ± 0.03 | 0.06 + 0.02 |

| OxPt | | | | OxPt/GEM/ |
|---|---|---|---|---|
| NCP | ZnP/SN38 | GMP NCP | OxPt/SN38^{b} | SN38^{b} |
| >200 | 5.95 ± 2.38 | 0.14 ± 0.05 | 1.67 ± 0.43 | 0.014 ± 0.004 |

^{a}Molar ratio for OxPt/SN38 had an OxPt:SN38 molar ratio of 1:2 and OxPt/GMP/SN38 had an OxPt:GMP:SN38 molar ratio of 2:1:4.
^{b}The IC50 values were calculated based on OxPt.

### In Vivo Toxicity and Efficacy:

The general toxicity of gemcitabine monophosphate nanoparticle (GMP NCP) was first tested on C57bl/6 mice on a Q3D dosing schedule. The mice tolerated GMP NCP at 2 mg/kg well for 15 doses. The maximum tolerated dose of GMP NCP was determined to be >2 mg/kg.

To investigate the general toxicity of three-drug nanoparticles, C57bl/6 mice were simultaneously dosed with OxPt/SN38 at 3.5 mg OxPt/kg and GMP NCP at 1, 1.25, 1.5, and 2 mg/kg GMP. The mice tolerated GMP NCP at loses up to 1.5 mg/kg for 10 doses while mice were found dead after three doses of GMP NCP at 2 mg/kg. A OxPt/GMP/SN38 with a chol-SN38:OxPt:GMP molar ratio of 4:2:1 was used for the subsequent anticancer efficacy study.

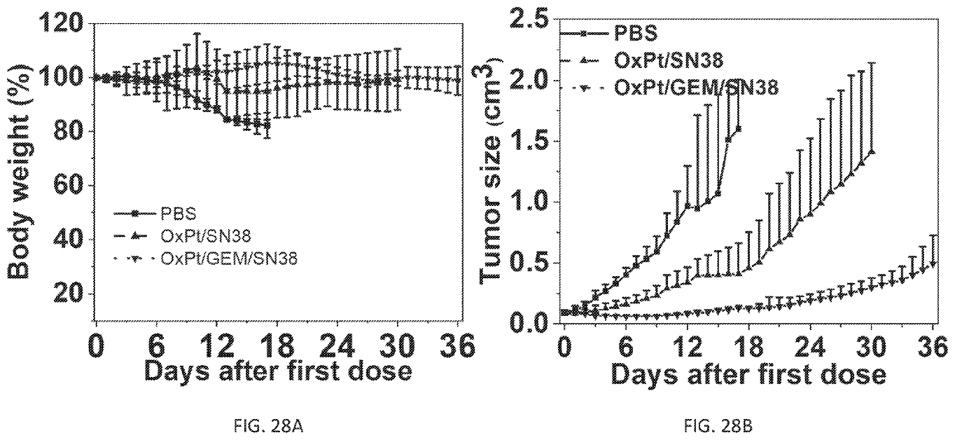

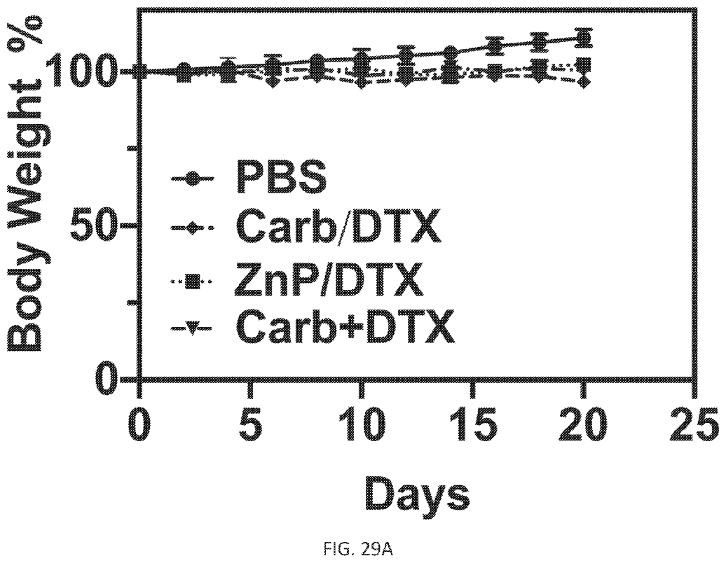

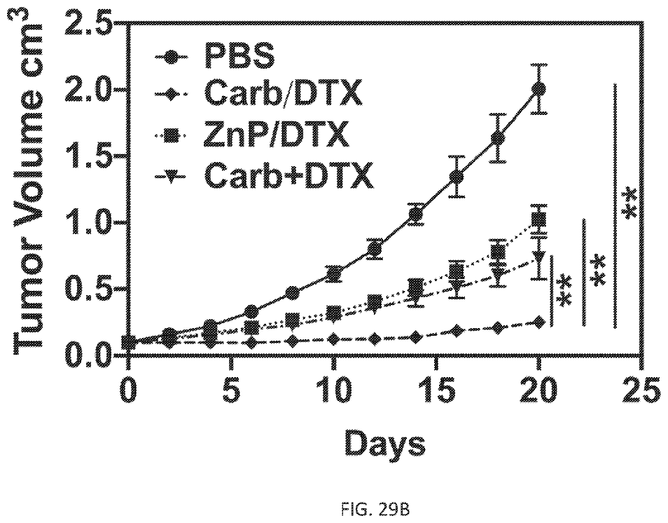

To determine the efficacy on pancreatic cancer models, 1×10⁶ KPC cells were inoculated on the right flanks of 6-week old C57bl/6 mice and the treatment started at day 7 when the sizes of tumors reached around 100 mm³. Mice were dosed with PBS, OxPt/SN38, OxPt/GEM/SN38 at an equivalent OxPt dose of 3.5 mg/kg on a Q3D schedule continuously dose until reaching the euthanasia endpoint. The effect of treatment on body weight is shown in FIG. 28A. As shown in FIG. 28B, OxPt/SN38 moderately inhibited tumor growth with a TGI of 74.4% at PBS endpoint. OxPt/GEM/SN38 showed a much higher antitumor efficacy with a TGI of 95.6%. This result indicates the strong synergy between three drugs in OxPt/GEM/SN38.

### Example 6

### NCP Core-Shell Particles Codeliver Carboplatin and Docetaxel

### Synthesis of Cholesterol-Docetaxel (Chol-DTX)

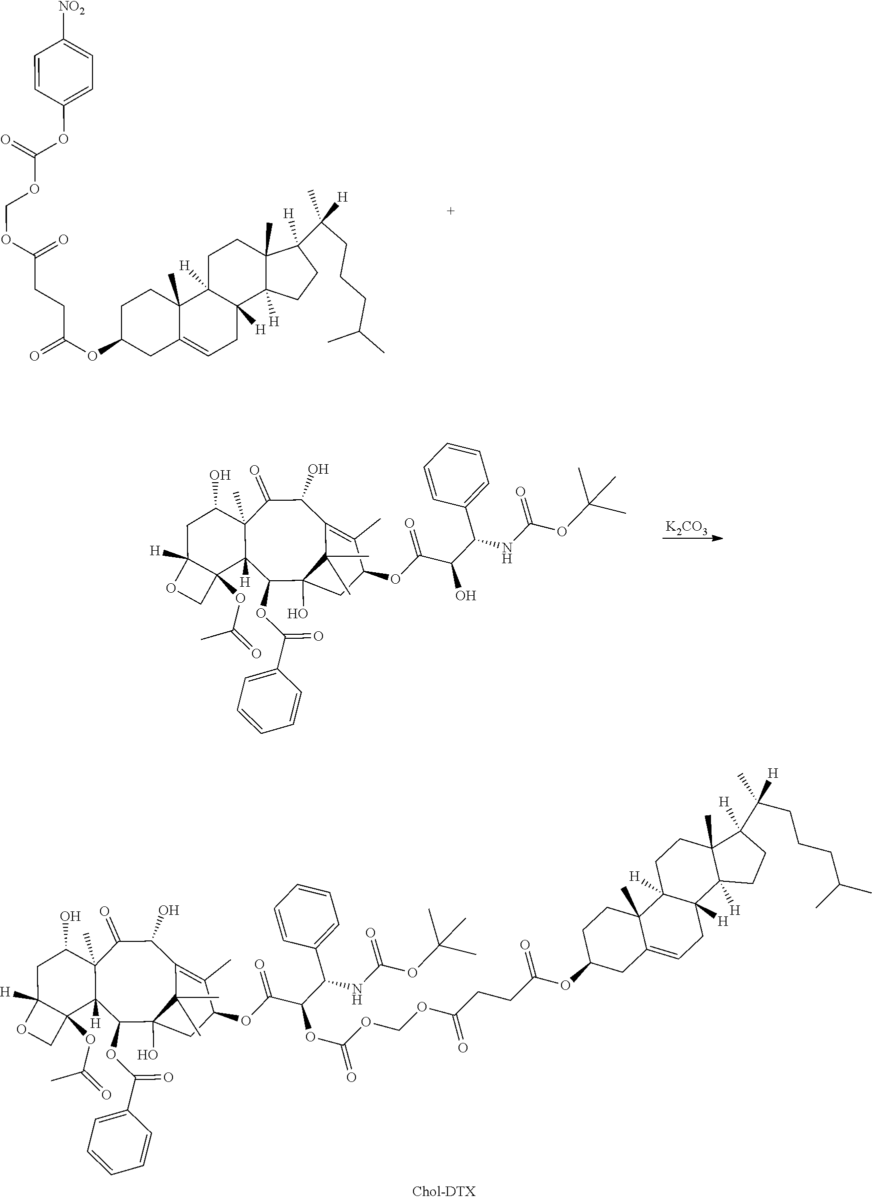

200 mg DTX (2.5 mmol, 1 eq) and 250 mg cholesterol linker (0.37 mmol, 1.5 eq) were dissolved in 20 mL acetonitrile in a 20 mL vial. 500 mg K₂CO₃ (3.6 mmol, 15 eq) was added to the vial and the mixture was stirred for 3 days until DTX completely disappeared. The mixture was diluted with 150 mL EtOAc and washed with 1M HCl and saturated NaCl, dried over Na₂SO₄ and evaporated on a rotary evaporator to obtain a colorless oil. The oil was purified by column chromatography using 2:1 Hexane: EtOAc to remove p-nitrophenol followed by 1:1 Hexane: EtOAc to elute the product. Yield: 180 mg (72%). ¹H NMR (400 MHz, CDCl3) δ 8.18-8.11 (m, 2H), 7.53 (t, J=7.6 Hz, 2H), 7.47-7.37 (m, 3H), 7.37-7.30 (m, 3H), 6.29 (s, 1H), 5.82-5.69 (m, 3H), 5.52-5.36 (m, 3H), 4.39-4.10 (m, 5H), 2.73-2.55 (m, 5H), 2.46 (s, 3H), 2.34 (t, J=9.4 Hz, 2H), 2.09-1.89 (m, 7H), 1.89-1.82 (m, 3H), 1.78 (s, 3H), 1.52 (dt, J=14.5, 7.4 Hz, 6H), 1.45-1.37 (m, 4H), 1.36 (s, 9H), 1.29 (d, J=7.2 Hz, 2H), 1.27 (s, 4H), 1.21-0.97 (m, 13H), 0.91 (ddd, J=17.3, 6.5, 2.3 Hz, 9H), 0.71 (d, J=8.6 Hz, 3H). HRMS: m/z=1367.7151 (expected 1367.7417 for [M+NH₄]⁺).

### Synthesis and Characterization of Carb DTX Core-Shell Nanoparticle

0.21 mg cholesterol, 0.42 mg DOPC, 0.75 mg DSPE-PEG2k, 0.25 mg chol-DTX and 0.5 mg bare Carb NCP particle (Poon.; et al. Mol. Pharmaceutics 2016, 13, 3665-3675) was mixed in 80 uL THF and added to 500 μL 30% EtOH with stirring at 1700 rpm stirring. The mixture was concentrated to 100 uL to obtain Carb/DTX core-shell nanoparticle. Z-average diameter and PDI of the Carb/DTX particle were 101.1 nm and 0.093 by DLS.

In Vitro Cytotoxicity of Carb DTX in Lung and Breast Cell Lines:

Human NSCLC H460 and Murine breast cancer 4T1 cells were seeded into 96-well plates at 2500 cells/well for 24 h. Carboplatin (Carb), DTX, Chol-DTX, Carb NCP (single drug), ZnP/DTX (single drug), Carb/DTX (two drugs, Carb:DTX molar ratio=1:1) at various concentrations were dosed to each well and the cells were incubated for another 48 h. The cell viability was measured by MTS assay.

As listed in Table 9, below, Carb and DTX have high cytotoxicity to H460 and 4T1 cells, while Chol-DTX is less toxic than DTX due to the need to release DTX to exert toxicity. Carb/DTX particle showed low an IC₅₀ value, indicating that there is a synergistic effect between Carb and Chol-DTX on the particle.

TABLE 9
Carb and DTX IC₅₀ values (μM) on H460 and 4T1 cells

| Cell lines | Carb | DTX | Chol-DTX | Carb/NPs | | ZnP/DTX | Carb/DTX |
|---|---|---|---|---|---|---|---|
| H460 | 3.90 (±0.39) | 3.69 (±0.74) | 11.37 (±2.37) | 28.09 | (±4.42) | 9.88 (±1.47) | 1.74 (±0.32) |
| 4T1 | 0.91 (±0.13) | 4.70 (±0.91) | 15.32 (±3.15) | 7.91 | (±1.72) | 3.08 (±0.58) | 0.93 (±0.17) |

Apoptosis/necrosis analysis was also performed on cells treated with 10 μM Carb and/or DTX by flow cytometry. Cells treated with Carb/DTX (molar ratio=1:1) showed much higher percentage of apoptosis (17.3% compared to 2.90% and 3.30% for Cab and DTX, respectively), which supports the synergistic effect between Carb and DTX delivered by the particle.

### In Vivo Antitumor Efficacy of Carb DTX:

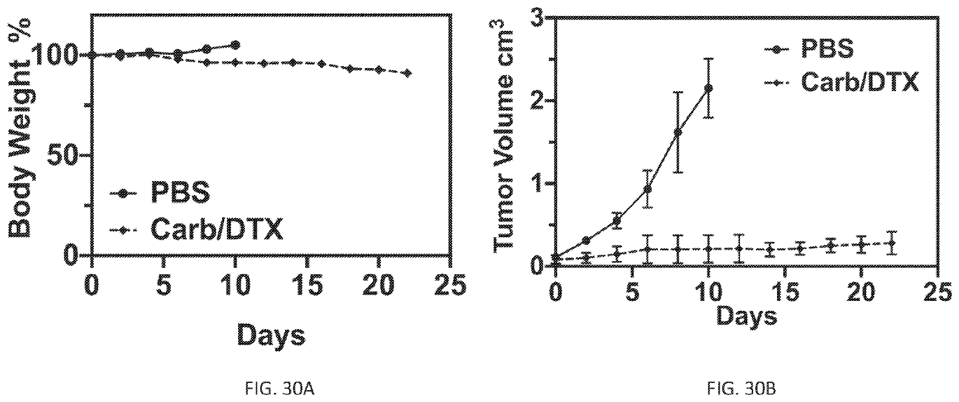

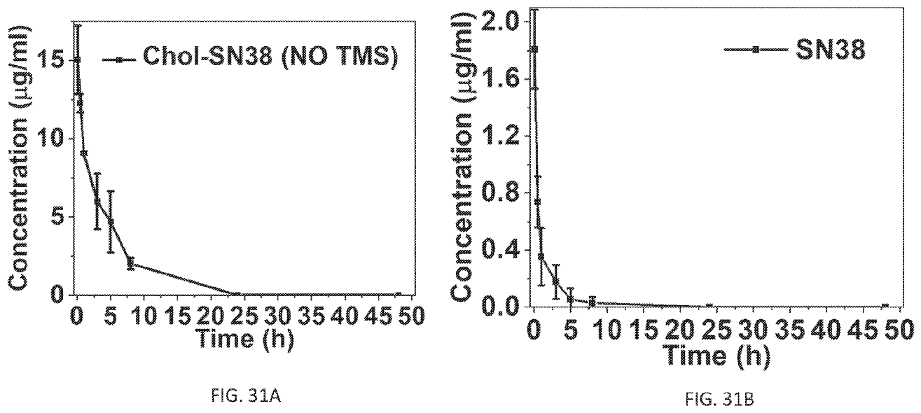

To determine in vivo antitumor efficacy, 1×10⁶ 4T1 cells were inoculated on the right flanks of 6-week old BALB/c mice and the treatment started at day 7 when the sizes of tumors reached around 100 mm³. Mice were dosed with PBS, free Carb plus DTX, ZnP/DTX or Carb/DTX at an equivalent Carb dose of 5 mg/kg once every week for 3 doses. As shown in FIGS. 29A and 29B, ZnP/DTX and Carb plus DTX moderately inhibited tumor growth with TGI values of 49.0% and 63.6%, respectively, at PBS endpoint. Carb/DTX showed a much better antitumor efficacy with a TGI of 87.5%. This result indicates the synergy of Carb and DTX delivered by the core-shell nanoparticle. As shown in FIGS. 30A and 30B, Carb/DTX was also highly effective inhibitor the growth of H₄₆₀ tumors.

### Example 7

NCP Core-Shell Particles Codeliver Oxaliplatin and Chol-SN38 (without TMS)

### Pharmacokinetics (PK) in Sprague Dawley Rats:

OxPt/SN38(no TMS) particles with an OxPt to chol-SN38(no TMS) with a molar ratio of 3:1 were made in a similar fashion as OxPt/SN38. OxPt/SN38(no TMS) was intravenously (i.v.) injected to three Sprague Dawley rats via tail veins at a dose of 2.0 mg OxPt/kg. 400 uL blood samples were collected at 5 min, 0.5 h, 1 h, 3 h, 5 h, 8 h, 24 h and 48 h. Blood samples was centrifuged at 10000 rpm for 10 min and plasma was collected for analysis. 200 μL metal-free concentrated nitric acid was added to 50 μL plasma and incubated for 48 h for complete digestion. Digested plasma was measured by ICP-MS for Pt concentration. Another 50 μL plasma was diluted by 100 μL saturated NaCl solution and 100 μL 0.5% triton X-100 water solution, then extracted with 200 μL ethyl acetate. The organic layer was analyzed by LC-HRMS to determine chol-SN38(no TMS) and SN38 concentrations. See FIGS. 31A and 31B.

As shown in Table 10, below, chol-SN38 on the particle surface layer showed a t_{1/2} of 3.4 h and an AUC_{0→∞} of 57.9 μg/ml*h. SN38 was detected in plasma with a half-life of 2.5 h, and AUC_{0→∞} values of 1.95 μg/ml*h. Thus, Chol-SN38(no TMS) quickly released SN38 and prolonged drug exposure when compared to SN38 from free irinotecan (half-life ˜1.8 h).

TABLE 10
Pharmacokinetics of OxPt/SN38 (No
TMS) in Sprague Dawley rats.

| Model type | Parameters | Unit | Chol-SN38 | SN38 |
|---|---|---|---|---|
| Non- | AUC_{0→inf} | (h · μg)/L | 57.9 ± 5.6 | 1.95 ± 0.30 |
| compartment | AUMC_{0→inf} | (h · h · μg)/L | 258.9 ± 56.1 | 3.83 ± 0.65 |
| model | CL | L/(h · kg) | 0.026 ± 0.003 | 0.35 ± 0.06 |
| | V_{SS} | L/kg | 0.116 ± 0.015 | 0.50 ± 0.09 |
| | MRT_{0→inf} | h | 4.46 ± 0.33 | 1.69 ± 0.65 |

### In Vivo Efficacy of OxPt SN38(No TMS):

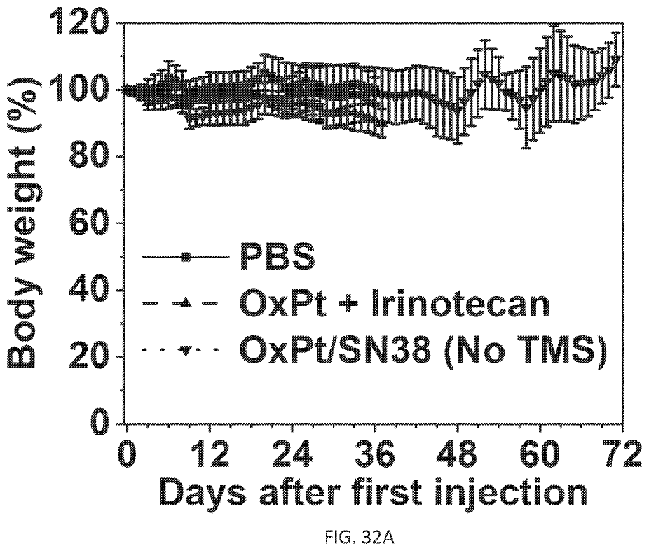

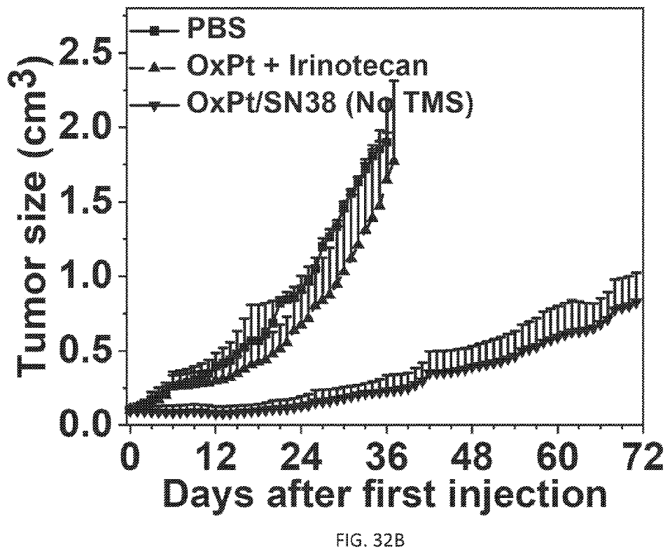

To determine the anticancer efficacy of OxPt/SN38(No TMS), 10×10⁶ HT29 cells were inoculated on the right flanks of 8-week old athymic nude mice and the treatment started at day 7 when the sizes of tumors reached around 100 mm³. Mice were dosed with PBS, OxPt plus irinotecan (3.5 mg/kg OxPt and 20.2 mg/kg irinotecan), or OxPt/SN38(No TMS) (3.5 mg/kg OxPt equivalent and 1.09 mg/kg SN38 equivalent) on a Q3D dosing schedule for 16 doses. As shown in FIGS. 32A and 32B, OxPt plus irinotecan showed minimal inhibition of tumor growth with a TGI of 13.4% at PBS endpoint. OxPt/SN38(No TMS) showed a much higher antitumor efficacy with a TGI of 87.9%. Notably, the SN38 content in irinotecan is 10.8 times than that in OxPt/SN38(No TMS). This result indicates the strong synergy between OxPt and SN38 delivered by the core-shell particle.

### Example 8

### Synthesis of Additional Cholesterol-Conjugated Paclitaxel (PTX) Prodrugs

### Synthesis of Chol-HBA-PTX-1

Chol-HBA-PTX-1 was synthesized in three steps starting from the cholesterol linker as shown below. 4-Hydroxylbenzyl alcohol was first coupled with cholesterol linker via the phenol group and then coupled with p-nitrophenol chloroformate. Then the new Chol-HBA linker was reacted with PTX in the presence of a base to yield Chol-HBA-PTX-1.

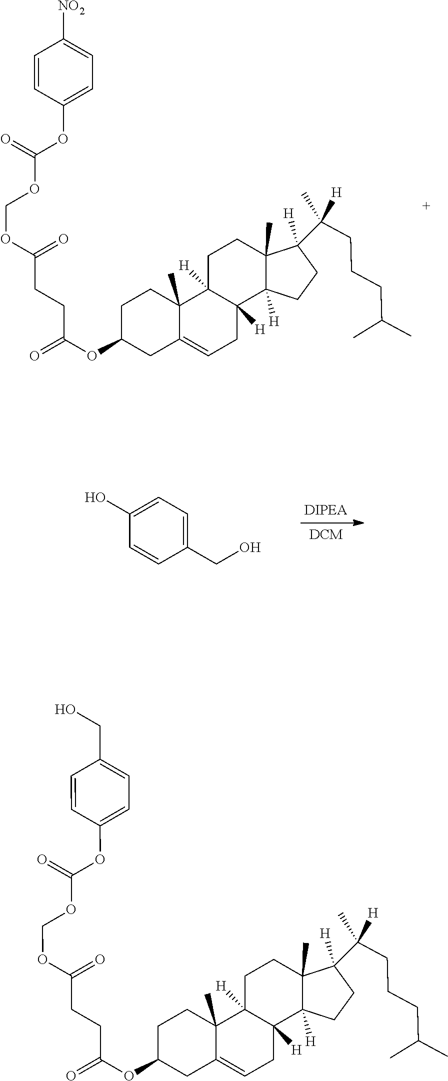 [SMILES: CC(C)CCC[C@@H](C)[C@H]1CC[C@H]2[C@@H]3CC=C4C[C@@H](OC(=O)CCC(=O)OCOC(=O)Oc5ccc([N+](=O)[O-])cc5)CC[C@]4(C)[C@H]3CC[C@]12C ; OCc1ccc(O)cc1 ; CC(C)CCC[C@@H](C)[C@H]1CC[C@H]2[C@@H]3CC=C4C[C@@H](OC(=O)CCC(=O)OCOC(=O)Oc5ccc(CO)cc5)CC[C@]4(C)[C@H]3CC[C@]12C]

0.68 g (1 mmol) cholesterol linker and 0.186 g 4-hydroxybenzyl alcohol (HBA, 1.5 mmol, 1.5 eq) was dissolved in 20 mL anhydrous DCM. 2.5 mL (3 mmol, 3 eq) DIPEA was added to the solution and the solution was stirred at room temperature under nitrogen protection for 24 h. The resulting solution was diluted with 20 mL DCM and washed with saturated NH₄Cl, saturated NaHCO₃, and saturated NaCl solution. The organic layer was then dried over anhydrous Na₂SO₄ and concentrated by rotary evaporator. The product was further purified by silica column chromatography eluted with 1:1 Hexane: EtOAc. Yield: 0.51 g (0.77 mmol, 77%). ¹H NMR (500 MHz, Chloroform-d) δ 7.40 (d, J=8.5 Hz, 2H), 7.20 (d, J=8.5 Hz, 2H), 5.87 (s, 2H), 5.41-5.32 (m, 1H), 4.70 (s, 2H), 4.63 (ddq, J=11.2, 7.7, 4.1 Hz, 1H), 2.73 (dd, J=7.0, 5.5 Hz, 2H), 2.65 (dd, J=6.9, 5.5 Hz, 2H), 2.35-2.29 (m, 2H), 2.09-0.61 (m, 43H).

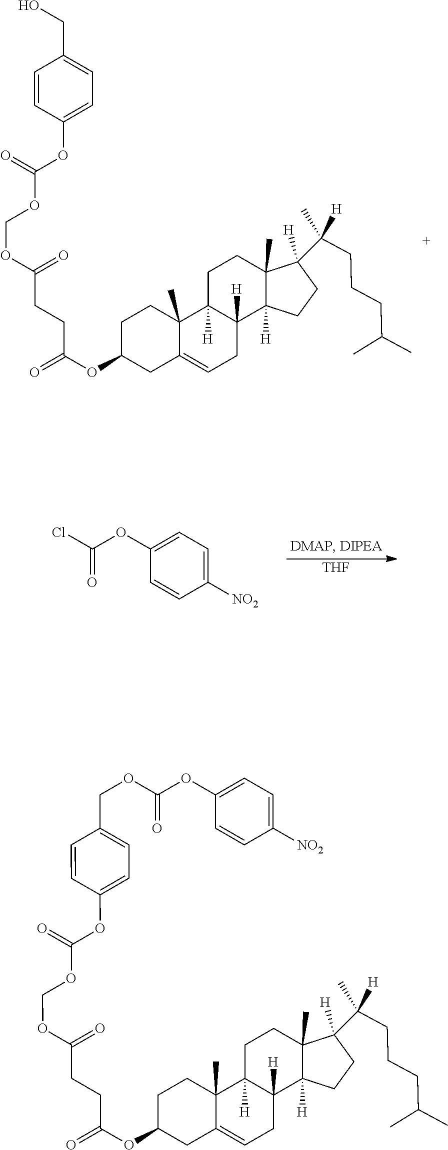

0.20 g (0.3 mmol, 1 eq) Chol-HBA, 150 uL DIPEA (0.9 mmol, 3 eq), and 7 mg DMAP (0.06 mmol, 0.2 eq) was dissolved in 5 mL anhydrous THF. 180 mg 4-nitrophenyl chloroformate in 5 mL anhydrous THF was dropwise added to the above solution under 0° C. and the solution was stirred at room temperature under nitrogen protection for 2 h. The resulting solution was diluted with 5 mL ethyl acetate and washed with saturated NH₄Cl, saturated NaHCO₃, and saturated NaCl solution. The organic layer was then dried over anhydrous Na₂SO₄ and concentrated by rotary evaporator. The product was further purified by silica column chromatography eluted with 4:1 Hexane: EtOAc. Yield: 0.125 g (0.15 mmol, 50%). ¹H NMR (500 MHz, Chloroform-d) δ 8.33-8.21 (m, 2H), 7.52-7.44 (m, 2H), 7.42-7.34 (m, 2H), 7.26 (d, J=8.6 Hz, 2H), 5.88 (s, 2H), 5.38-5.34 (m, 1H), 5.29 (s, 2H), 4.63 (td, J=15.3, 13.3, 7.7 Hz, 1H), 2.76-2.70 (m, 2H), 2.66 (dd, J=7.0, 5.4 Hz, 2H), 2.32 (d, J=7.8 Hz, 2H), 2.06-0.65 (m, 43H).

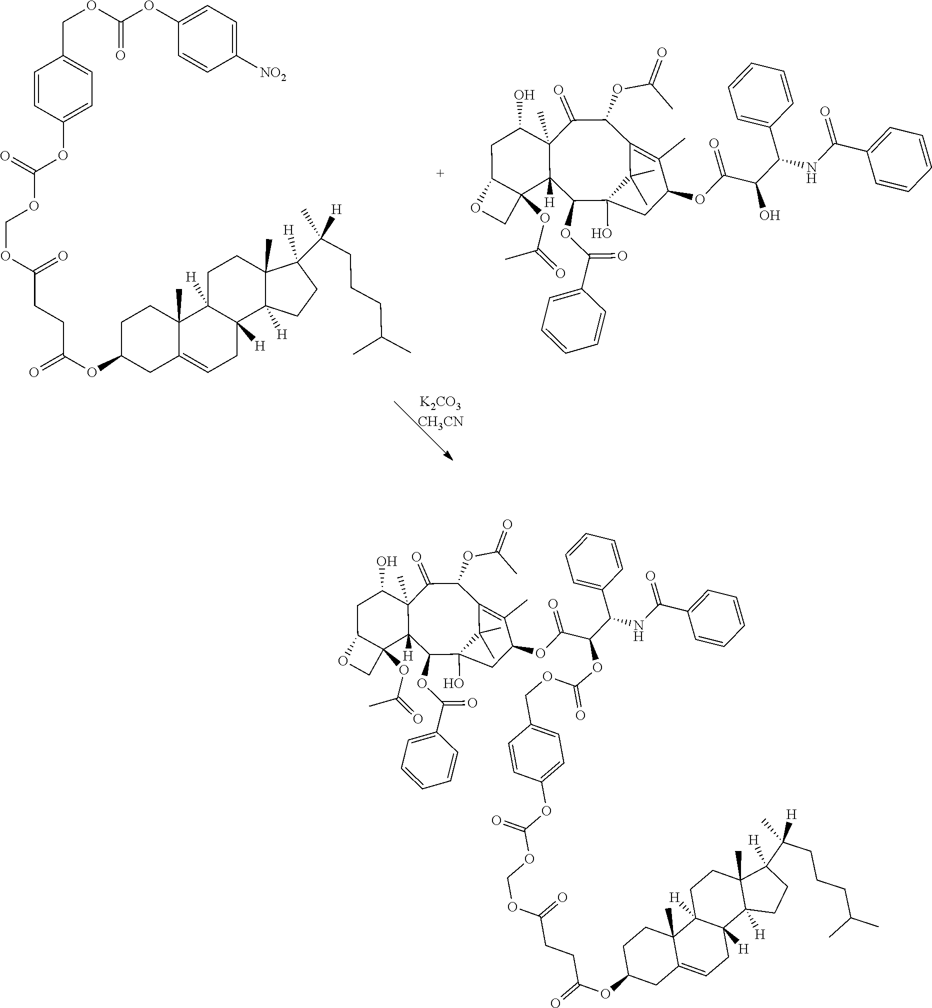

0.10 g (0.15 mmol) Chol-HBA linker and 0.125 g PTX (0.15 mmol, 1.0 eq) was dissolved in 10 mL anhydrous CH₃CN. 0.3 g K₂C₀₃ (4.5 mmol, 30 eq) was added to the solution and the solution was stirred at room temperature under nitrogen protection for 48 h.

The resulting solution was diluted with 20 mL ethyl acetate and washed with saturated NH₄Cl, saturated NaHCO₃, and saturated NaCl solution. The organic layer was then dried over anhydrous Na₂SO₄ and concentrated with a rotary evaporator. The product was further purified by silica column chromatography eluted with 1:1 Hexane: EtOAc. Yield: 55 mg (0.036 mmol, 24%). ¹H NMR (500 MHz, Chloroform-d) δ 8.18-8.12 (m, 2H), 7.78-7.69 (m, 3H), 7.61 (t, J=7.3 Hz, 2H), 7.55-7.45 (m, 4H), 7.43-7.33 (m, 6H), 7.23-7.19 (m, 2H), 6.29 (d, J=4.8 Hz, 3H), 5.99 (dd, J=9.3, 2.7 Hz, 1H), 5.87 (s, 2H), 5.69 (d, J=6.9 Hz, 1H), 5.44 (d, J=2.7 Hz, 1H), 5.36 (d, J=5.1 Hz, 1H), 5.16 (d, J=4.4 Hz, 1H), 4.97 (t, J=7.7 Hz, 2H), 4.50-4.38 (m, 1H), 4.32 (d, J=8.4 Hz, 2H), 4.22 (s, 1H), 3.81 (t, J=8.7 Hz, 1H), 2.73 (t, J=6.4 Hz, 2H), 2.65 (t, J=6.4 Hz, 2H), 2.61-0.65 (m, 67H). HRMS: m/z=1546.7330 ([M+H]⁺).

### Synthesis of Chol-HBA-PTX-2

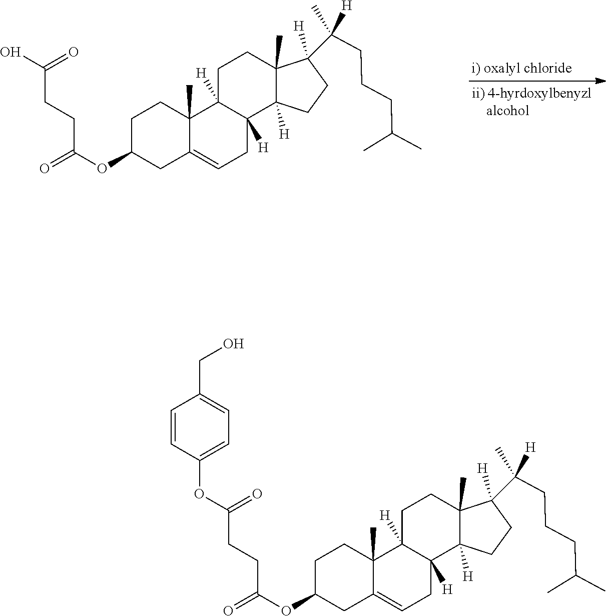 [SMILES: CC(C)CCC[C@@H](C)[C@H]1CC[C@H]2[C@@H]3CC=C4C[C@@H](OC(=O)CCC(=O)O)CC[C@]4(C)[C@H]3CC[C@]12C ; CC(C)CCC[C@@H](C)[C@H]1CC[C@H]2[C@@H]3CC=C4C[C@@H](OC(=O)CCC(=O)Oc5ccc(CO)cc5)CC[C@]4(C)[C@H]3CC[C@]12C]

In a dry round bottom flask, cholesteryl hydrogen succinate (0.486 g, 1 mmol, 1 eq) was dissolved in 20 mL anhydrous CH₂Cl₂, followed by slow addition of oxalyl chloride (3 eq). The reaction mixture was stirred under inert atmosphere under room temperature for 2 h. The solution was concentrated under vacuum to remove the solvent, and further dried with extra anhydrous CH₂Cl₂ (20 mL×3) to remove the unreacted oxalyl chloride. The crude cholesteryl succinate chloride was then dissolved in 20 mL of anhydrous THF, and 4-hydroxylbenzyl alcohol (0.186 g, 1.5 mmol, 1.5 eq) was added to the solution subsequently. The mixture solution was kept stirring overnight under room temperature and monitored by TLC. After the reaction complete, the resulting solution was diluted with 20 mL ethyl acetate and washed with saturated NH₄Cl, saturated NaHCO₃, and saturated NaCl solution. The organic layer was then dried over anhydrous Na₂SO₄ and concentrated by rotary evaporator. The product was further purified by silica column chromatography eluted with Hexane/EtOAc. Yield: 0.45 g (0.77 mmol, 77%). ¹H NMR (500 MHz, Chloroform-d) δ 7.32 (d, J=8.5 Hz, 2H), 7.05 (d, J=8.5 Hz, 2H), 5.36 (dd, J=4.7, 2.0 Hz, 1H), 4.72-4.55 (m, 3H), 2.84 (dd, J=7.5, 6.0 Hz, 2H), 2.69 (dd, J=7.6, 6.0 Hz, 2H), 2.39 (s, 1H), 2.32 (d, J=7.9 Hz, 2H), 2.06-0.61 (m, 41H).

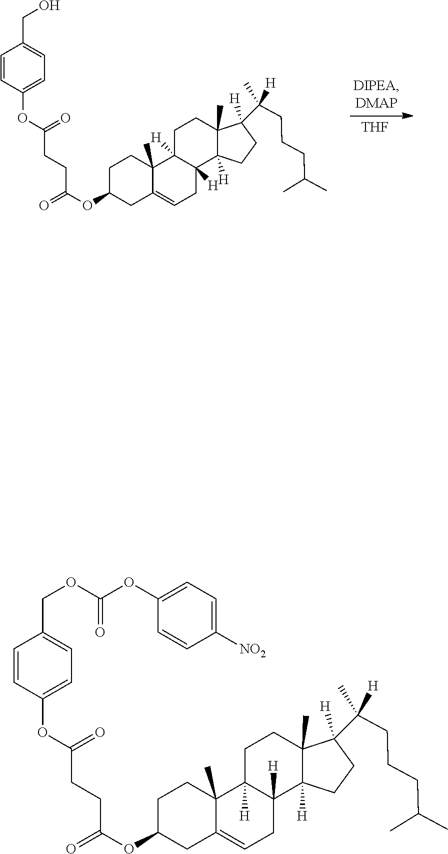

0.18 g (0.3 mmol, 1 eq) cholesteryl succinate 4-hydroxylbenzyl alcohol ester, 150 uL DIPEA (0.9 mmol, 3 eq), and 7 mg DMAP (0.06 mmol, 0.2 eq) was dissolved in 5 mL anhydrous THF. 180 mg 4-nitrophenyl chloroformate in 5 mL anhydrous THF was dropwise added to the above solution under 0° C. and the solution was stirred at room temperature under nitrogen protection for 2 h. The resulting solution was diluted with 5 mL ethyl acetate and washed with saturated NH₄Cl, saturated NaHCO₃, and saturated NaCl solution. The organic layer was then dried over anhydrous Na₂SO₄ and concentrated by rotary evaporator. The product was further purified by silica column chromatography eluted with Hexane/EtOAc. Yield: 0.150 g (0.2 mmol, 67%). ¹H NMR (500 MHz, Chloroform-d) δ 8.24-8.20 (m, 2H), 7.47-7.42 (m, 2H), 7.40-7.34 (m, 2H), 7.18-7.07 (m, 2H), 5.36 (dd, J=4.9, 1.8 Hz, 1H), 5.27 (s, 2H), 2.88 (dd, J=7.6, 5.8 Hz, 2H), 2.72 (dd, J=7.6, 5.8 Hz, 2H), 2.38-2.28 (m, 2H), 2.05-0.62 (m, 41H).

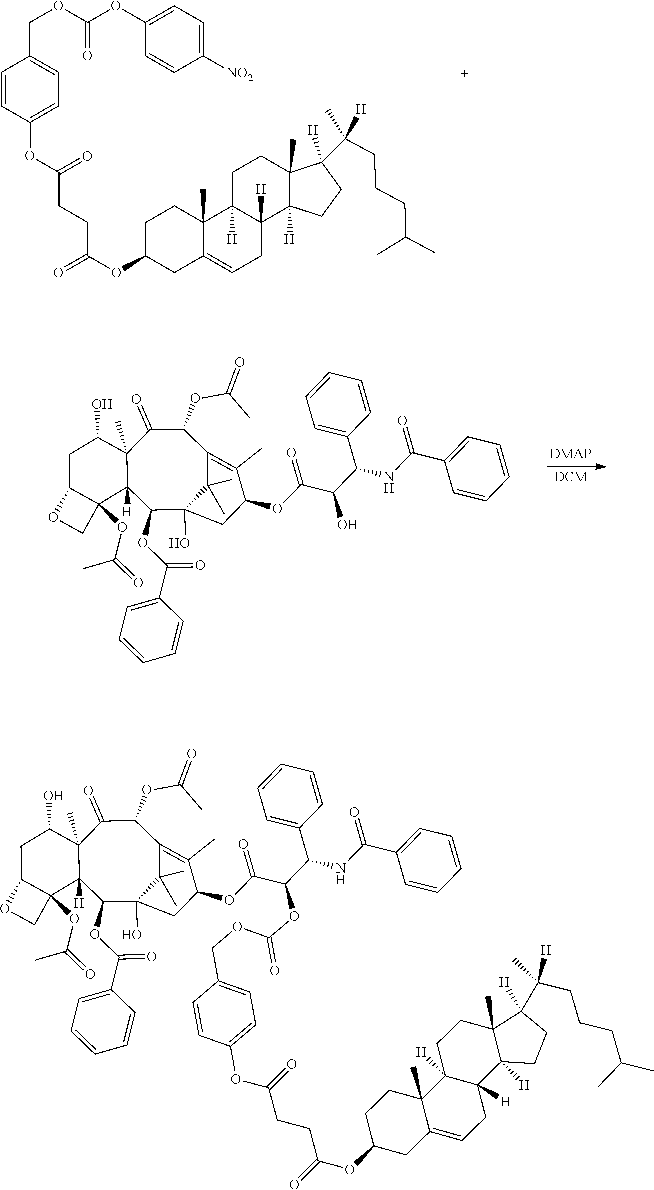

0.11 g (0.15 mmol) Chol-HBA linker 2 and 0.125 g PTX (0.15 mmol, 1.0 eq) was dissolved in 10 mL anhydrous CH₂Cl₂. 17 mg DMAP (0.15 mmol, 1 eq) was added to the solution and the solution was stirred at room temperature under nitrogen protection for 48 h.

The resulting solution was diluted with 20 mL ethyl acetate and washed with saturated NH₄Cl, saturated NaHCO₃, and saturated NaCl solution. The organic layer was then dried over anhydrous Na₂SO₄ and concentrated by rotary evaporator. The product was further purified by silica column chromatography eluted with 1:1 Hexane: EtOAc. Yield: 102 mg (0.07 mmol, 470%). ¹H NMR (500 MHz, Chloroform-d) δ 8.18-8.08 (m, 2H), 7.77-7.70 (m, 2H), 7.61 (t, J=7.4 Hz, 1H), 7.54-7.45 (m, 3H), 7.42-7.34 (m, 8H), 7.10 (d, J=8.5 Hz, 2H), 6.90 (d, J=9.3 Hz, 1H), 6.30 (s, 1H), 5.99 (dd, J=9.3, 2.7 Hz, 1H), 5.69 (d, J=7.1 Hz, 1H), 5.46-5.36 (m, 2H), 5.15 (d, J=3.4 Hz, 2H), 4.98 (d, J=9.5 Hz, 1H), 4.44 (s, 1H), 4.39-4.32 (m, 2H), 4.21 (d, J=8.4 Hz, 1H), 3.82 (d, J=7.0 Hz, 1H), 2.88 (t, J=6.7 Hz, 2H), 2.72 (t, J=6.5 Hz, 2H), 2.66-0.58 (m, 67H).

Additional examples of lipid-conjugated prodrugs are shown in FIGS. 33, 34, 35A, and 35B.

It will be understood that various details of the presently disclosed subject matter may be changed without departing from the scope of the presently disclosed subject matter.

Furthermore, the foregoing description is for the purpose of illustration only, and not for the purpose of limitation.

1. A prodrug comprising a structure of the formula D-BL-L, wherein
D is a monovalent drug moiety, optionally wherein D is a monovalent derivative of an anti-cancer drug compound, further optionally wherein D is a monovalent derivative of a drug compound selected from the group consisting of Etoposide (ET), Podophyllotoxin (PPX), Paclitaxel (PTX), Docetaxel (DTX), dihydroartemisin (DHA), Camptothecin (CPT), 7-ethyl-10-hydroxycamptothecin (SN38), Topotecan, Doxorubicin, Epirubicin, Idarubicin, Vincristine, Mitoxantrone, Artesunate, Capecitabine, Octreotide, Leuprolide, and Goserelin;
L is a monovalent lipid moiety; and
BL is a bivalent linker, wherein D is directly attached to BL via a carbonate or carbamate group, and wherein BL comprises at least one of an acetal group and a substituted oxybenzyloxy group, wherein the acetal group has a structure of one of the formulas: 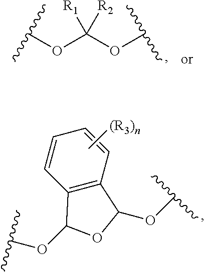
wherein:
n is an integer between 0 and 4, optionally wherein n is 0;
R₁ and R₂ are independently selected from the group consisting of H, alkyl, substituted alkyl, aralkyl, substituted aralkyl, aryl, and substituted aryl,
each R₃ is independently selected from the group consisting of alkyl, aralkyl, aryl, a halo, alkoxy, aryloxy, hydroxy, acyl, carboxylate, phosphate, nitro, —N₃, B(OH)₂, and cyano; and
wherein an oxygen atom of the acetal group is directly attached to a carbon atom of a carbonate or carbamate group; and
wherein the substituted oxybenzyloxy group has a structure of the formula: 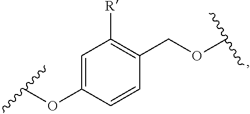
wherein R′ is selected from the group consisting of nitro, N₃, and —B(OH)₂, and wherein the oxygen atom attached to the benzyl carbon of the oxybenzyloxy group is directly attached to a carbon atom of a carbonate or carbamate group.

2. The prodrug of claim 1, wherein L is a monovalent derivative of cholesterol, oleic acid, a lyso-lipid, or phosphocholine.

3. The prodrug of claim 1, wherein the prodrug comprises a structure of one of formulas:

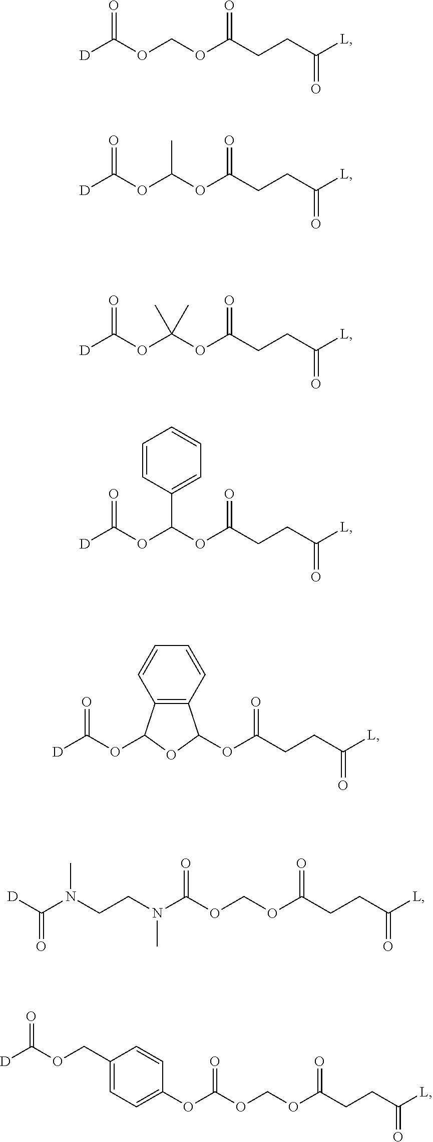

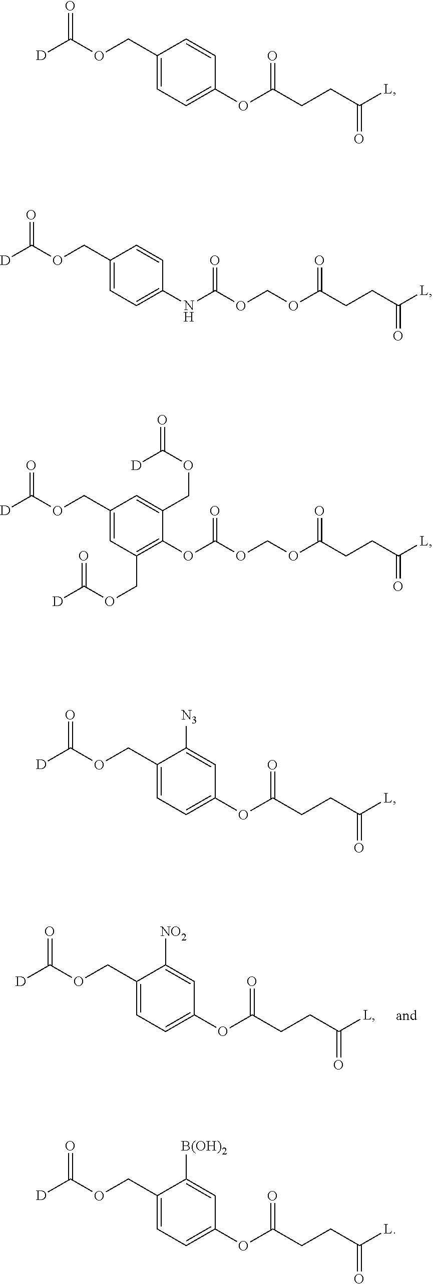

4. The prodrug of claim 1, wherein BL comprises an acetal group having a structure of the formula: 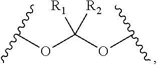
wherein R₁ and R₂ are independently selected from the group consisting of H, alkyl, substituted alkyl, aralkyl, substituted aralkyl, aryl, and substituted aryl; optionally wherein R₁ and R₂ are independently selected from the group consisting of H, methyl, and phenyl; further optionally wherein both R₁ and R₂ are H.

5. The prodrug of claim 4, wherein L is an oleic acid moiety and wherein L and BL together have the structure:

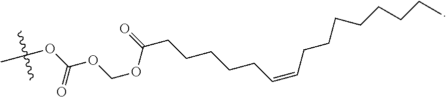 [SMILES: *OC(=O)OCOC(=O)CCCCC/C=C\CCCCCCCC]

6. The prodrug of claim 4, wherein L is a cholesterol derivative and where L and BL together have the structure:

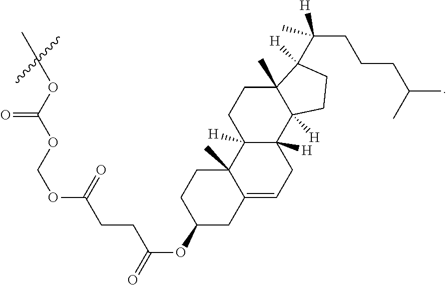 [SMILES: *OC(=O)OCOC(=O)CCC(=O)O[C@H]1CC[C@@]2(C)C(=CC[C@H]3[C@@H]4CC[C@H]([C@H](C)CCCC(C)C)[C@@]4(C)CC[C@@H]32)C1]

7. The prodrug of claim 6, wherein the prodrug is selected from the group consisting of:

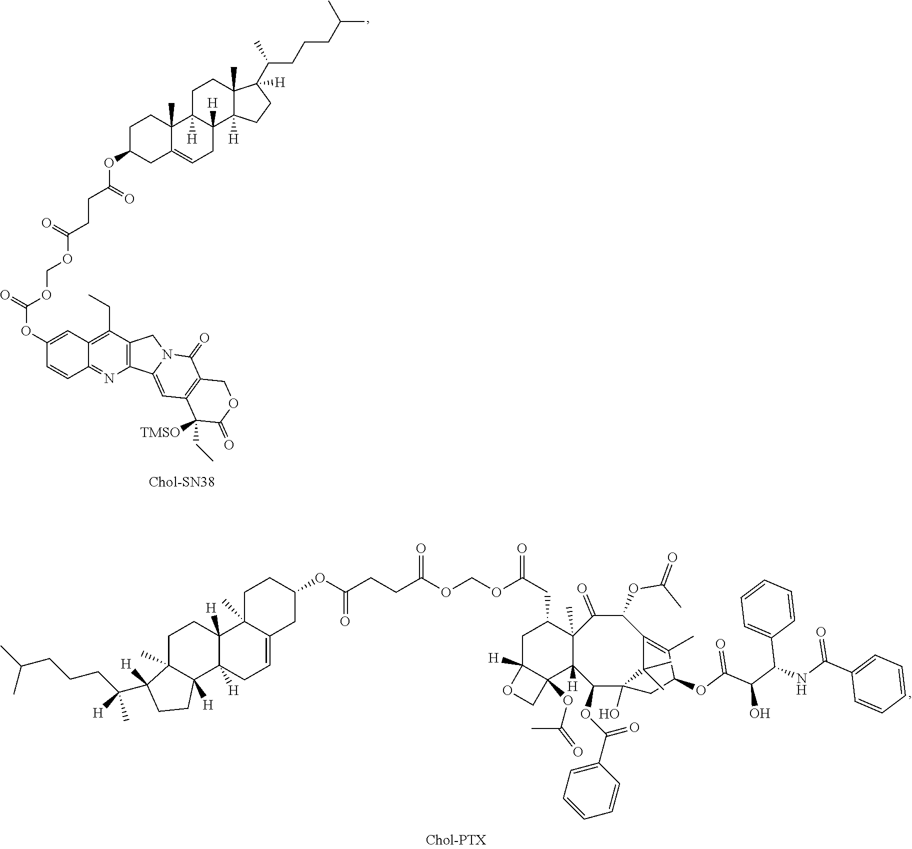

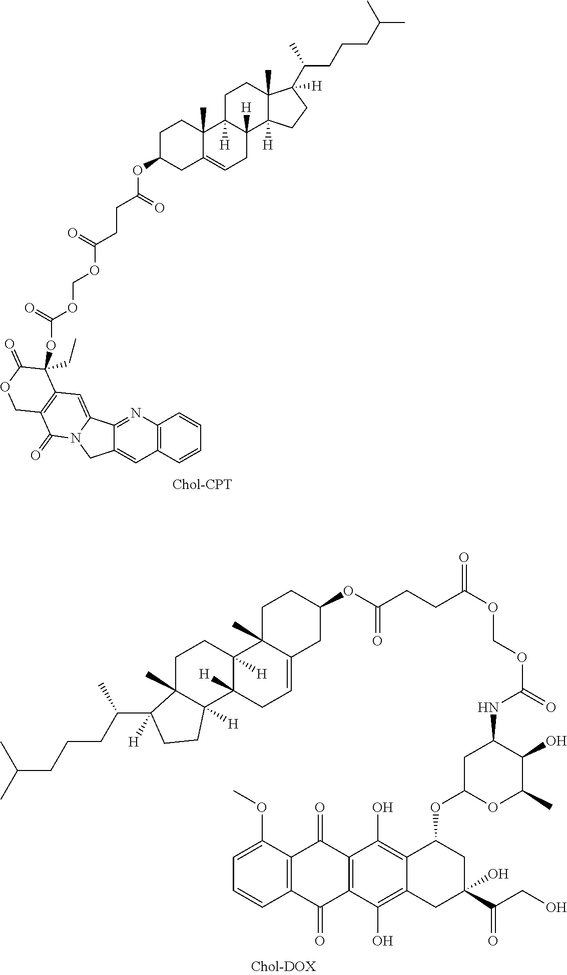

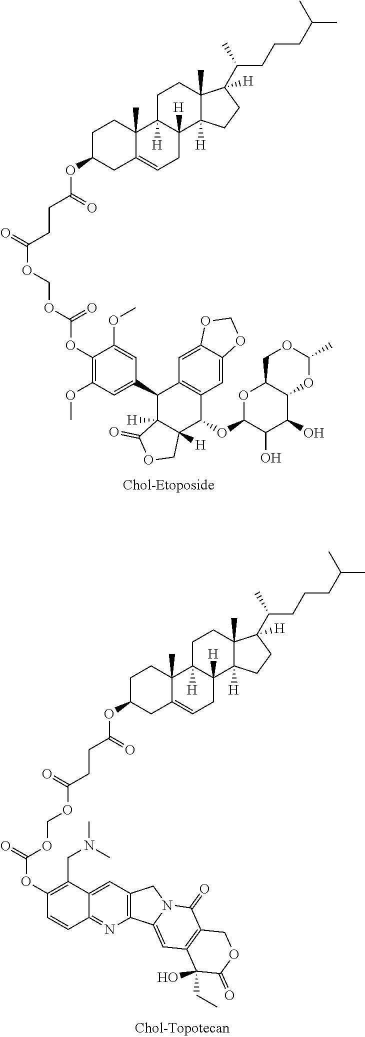

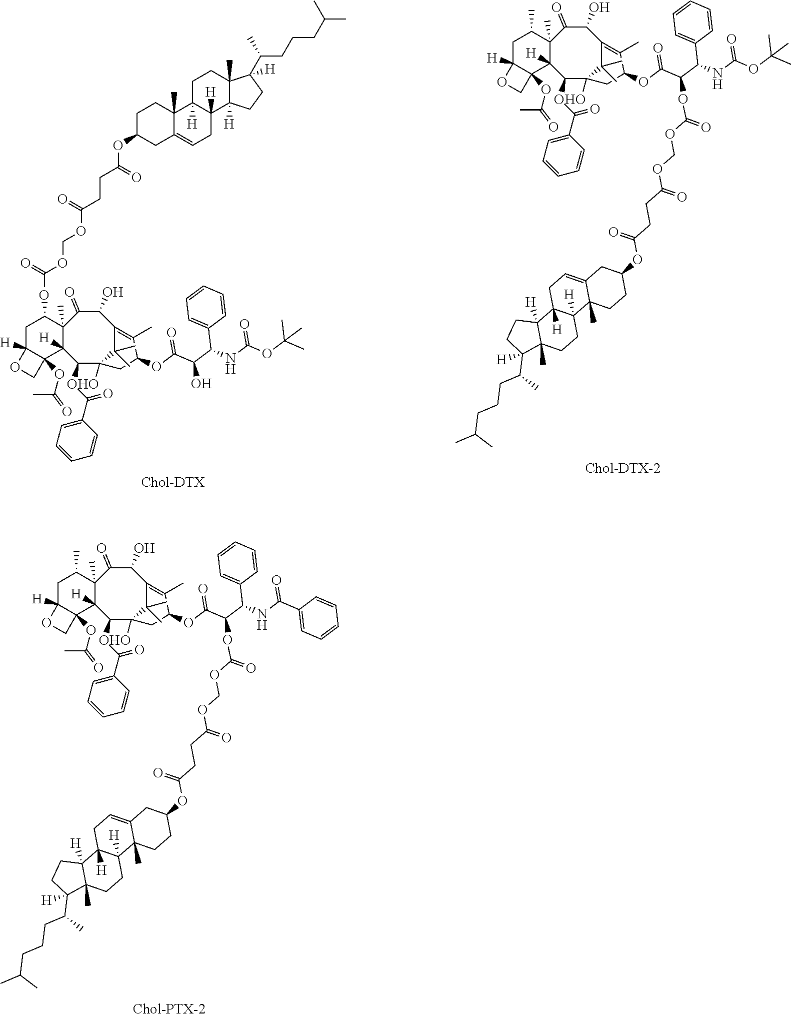

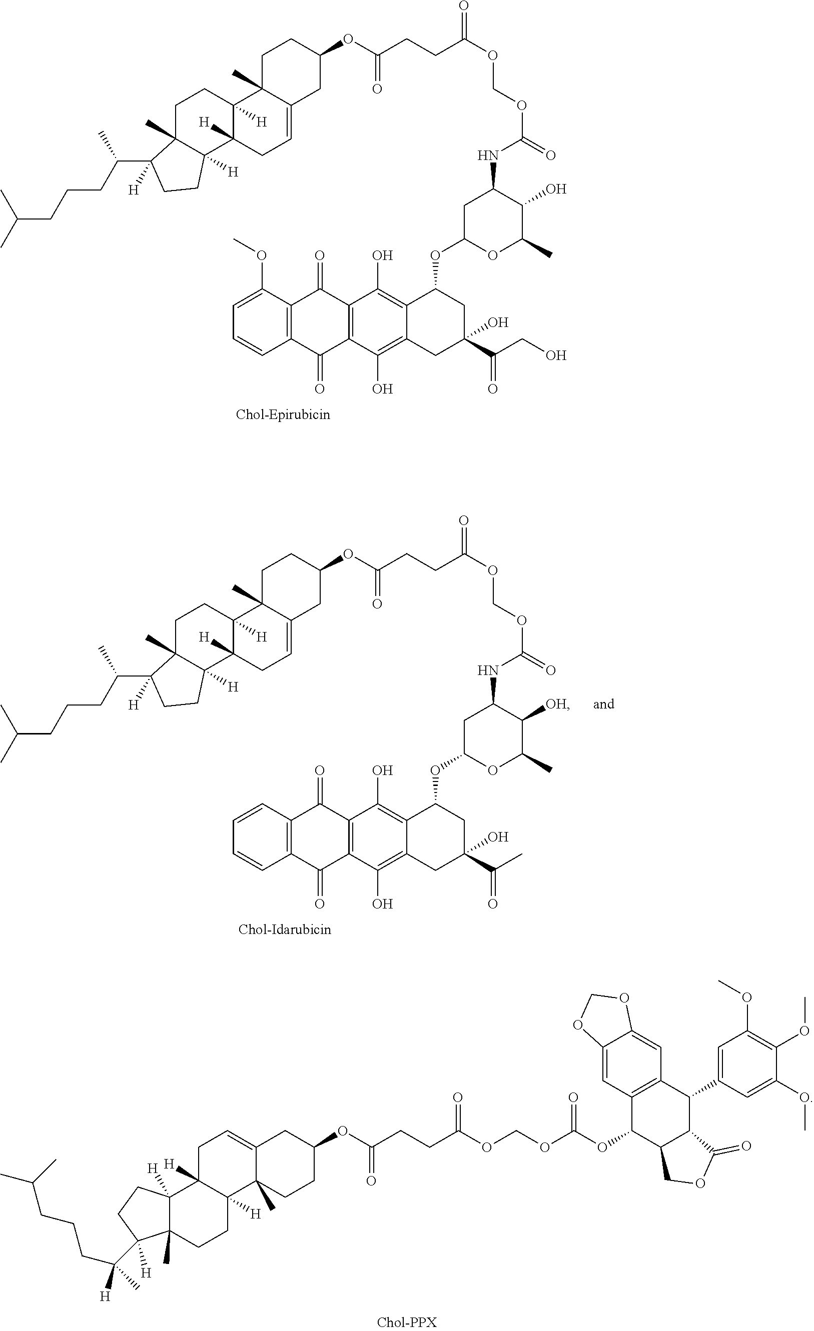

8. The prodrug of claim 1, wherein the prodrug binds to low-density lipoprotein (LDL) and is actively transported to tumors via LDL-receptor mediated endocytosis, optionally wherein the prodrug has an association constant Kₐ for LDL that is at least about 1000 times the Kₐ of the prodrug for albumin, further optionally wherein the prodrug has a Kₐ for LDL that is at least about 2000 times that of the Kₐ of the prodrug for albumin.

9. A nanoparticle comprising:
(a) a core comprising a metal-organic matrix material, optionally wherein the metal-organic matrix material comprises a coordination polymer; and
(b) a coating layer covering at least a portion of the surface of the core, wherein said coating layer comprises a lipid layer or a lipid bilayer and wherein said coating layer comprises one or more prodrug of claim 1.

10. The nanoparticle of claim 9, wherein the metal-organic matrix material comprises a nanoscale coordination polymer comprising a metal bisphosphate comprising a multivalent metal ion and a bisphosphate, optionally wherein the multivalent metal ion is selected from the group consisting of Ca²⁺, Mg²⁺, Mn²⁺, Zn²⁺ and combinations thereof.

11. The nanoparticle of claim 10, wherein the bisphosphate comprises a prodrug of an anti-cancer agent, optionally wherein the bisphosphate comprises a cisplatin, carboplatin or oxaliplatin prodrug, further optionally wherein the bisphosphate is a bisphosphate ester of cis, cis-trans-[Pt(NH₃)₂Cl₂(OH)₂] or cis, trans-[Pt(dach)(oxalate)(OH)₂].

12. The nanoparticle of claim 9, wherein the core comprises an embedded anti-cancer agent, optionally an embedded hydrophilic anti-cancer agent, further optionally wherein the embedded anti-cancer agent is gemcitabine monophosphate (GMP).

13. The nanoparticle of claim 9, wherein the core comprises at least two anti-cancer agents, optionally wherein the at least two anti-cancer agents comprise a first anti-cancer agent, wherein the first anti-cancer agent is a cisplatin, carboplatin or oxaliplatin prodrug, further optionally a bisphosphate of cisplatin, carboplatin or oxaliplatin; and a second anti-cancer agent, wherein the second anti-cancer agent is an embedded, hydrophilic anti-cancer agent.

14. The nanoparticle of claim 9, wherein the nanoparticle core comprises a metal bisphosphate coordination polymer comprising a multivalent metal ion, optionally Zn²⁺, and a bisphosphate, wherein said bisphosphate is an oxaliplatin prodrug having the structure Pt(dach)(oxalate)(bisphosphoramidic acid); and wherein the coating layer is a lipid bilayer comprising a prodrug having the structure:

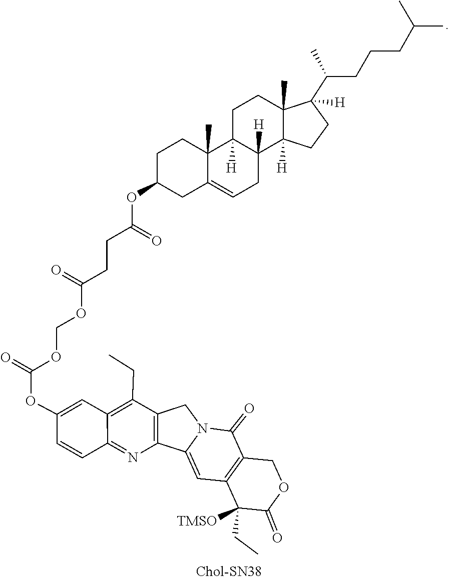

15. The nanoparticle of claim 14, wherein the nanoparticle core further comprises GMP embedded in the nanoparticle core.

16. The nanoparticle of claim 9, wherein the coating layer comprises a lipid bilayer comprising a cationic lipid and/or a functionalized lipid, wherein said functionalized lipid is a lipid functionalized with a group that can bond to a nucleic acid, and wherein at least one nucleic acid is covalently bonded to the functionalized lipid or attached to the cationic lipid via electrostatic interactions, optionally wherein said lipid bilayer comprises a mixture comprising one or more of a thiol- or dithiol-functionalized 1,2-distearoyl-sn-glycero-3-phosphoethanolamine (DSPE), 1,2-dioleoyl-3-trimethylammonium propane (DOTAP), and 1,2-dioleoyl-sn-glycero-3-phosphocholine (DOPC).

17. The nanoparticle of claim 16, wherein the at least one nucleic acid is selected from the group consisting of a siRNA, a miRNA, and an AS ODN, optionally wherein the siRNA is selected from the group consisting of survivin siRNA, ERCC-1 siRNA, P-glycoprotein siRNA (P-gp siRNA), Bcl-2 siRNA, and a mixture thereof.

18. The nanoparticle of claim 9, wherein the nanoparticle further comprises one or more passivating agents, optionally a hydrophilic polymer; a targeting agent, optionally a RGD peptide; and an immunotherapy agent.

19. The nanoparticle of claim 9, wherein the nanoparticle has a diameter ranging from about 20 nanometers to about 140 nanometers.

20. The nanoparticle of claim 9, wherein the nanoparticle adsorbs plasma proteins, optionally apolipoprotein B-100, for active transport to tumors via LDL receptor-mediated endocytosis.

21. A pharmaceutical formulation comprising (i) a pharmaceutically acceptable carrier and (ii) a prodrug of claim 1.

22. A method of treating cancer in a subject in need thereof, wherein the method comprises administering to the subject a prodrug of claim 1.

23. The method of claim 22, wherein the method further comprises administering to the subject an additional cancer treatment selected from the group consisting of surgery, radiotherapy, chemotherapy, toxin therapy, immunotherapy, cryotherapy and gene therapy; optionally wherein the additional cancer treatment is immunotherapy.

24. The method of claim 23, wherein the immunotherapy comprises administering to the subject an immunotherapy agent; optionally wherein the immunotherapy agent is selected from the group consisting of an anti-CD52 antibody, an anti-CD20 antibody, an anti-CD47 antibody an anti-GD2 antibody, a cytokine, polysaccharide K; a PD-1 inhibitor, a PD-L1 inhibitor, a CTLA-4 inhibitor, an IDO inhibitor, a CCR7 inhibitor, an OX40 inhibitor, a TIM3 inhibitor, and a LAG3 inhibitor.

25. The method of claim 22, wherein the cancer is selected from the group consisting of a head tumor, a neck tumor, breast cancer, a gynecological tumor, a brain tumor, colorectal cancer, lung cancer, mesothelioma, a soft tissue sarcoma, skin cancer, connective tissue cancer, adipose cancer, lung cancer, stomach cancer, anogenital cancer, kidney cancer, bladder cancer, colon cancer, prostate cancer, central nervous system cancer, retinal cancer, blood cancer, neuroblastoma, multiple myeloma, lymphoid cancer, and pancreatic cancer.

26. The method of claim 22, wherein the cancer is a metastatic cancer, optionally a metastatic colorectal cancer.

27-31. (canceled)

32. A pharmaceutical formulation comprising (i) a pharmaceutically acceptable carrier and (ii) a nanoparticle of claim 9.

33. A method of treating cancer in a subject in need thereof, wherein the method comprises administering to the subject a nanoparticle of claim 9.

34. The method of claim 33, wherein the nanoparticle core comprises a metal bisphosphate coordination polymer comprising a multivalent metal ion, optionally selected from Ca²⁺, Mg²⁺, Mn²⁺, Zn²⁺ and combinations thereof, and a bisphosphate, wherein said bisphosphate is a bisphosphate ester of cisplatin, oxaliplatin or carboplatin; and
wherein the coating layer comprises a lipid bilayer comprising a prodrug having the structure D-BL-L wherein D is a monovalent drug moiety of an anti-cancer drug compound, optionally wherein the monovalent drug moiety is a monovalent derivative of a drug compound selected from the group consisting of ET, PPX, PTX, DTX, DHA, CPT, SN38, Topotecan, Doxorubicin, Epirubicin, Idarubicin, Vincristine, Mitoxantrone, Artesunate, Capecitabine, Octreotide, Leuprolide, and Goserelin; L is a monovalent lipid moiety, optionally a monovalent cholesterol moiety; and BL is a bivalent linker moiety wherein D is attached to BL via a carbonate or a carbamate bond, and wherein BL comprises an acetal group, wherein the acetal group has a structure of the formula: 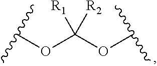
wherein R₁ and R₂ are independently selected from the group consisting of H, alkyl, substituted alkyl, aralkyl, substituted aralkyl, aryl, and substituted aryl; and wherein at least one of the oxygen atoms in the acetal group is directly bonded to a carbon atom of a carbonate or carbamate group.

35. The method of claim 34, wherein the nanoparticle core further comprises a hydrophilic anti-cancer agent embedded therein, optionally wherein the hydrophilic anti-cancer agent is GMP.

36. The method of claim 34, wherein the prodrug has a structure of the formula: 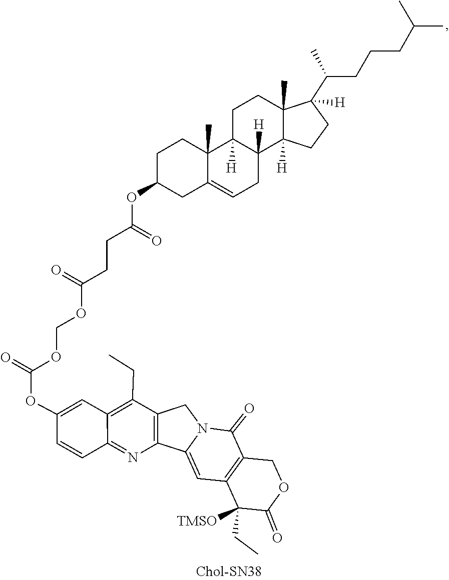
optionally wherein the bisphosphate is Pt(dach)(oxalate)(bisphosphoramidic acid).

37. The method of claim 34, wherein the method further comprises administering to the subject an immunotherapy agent.

38. The method of claim 34, wherein administration of the nanoparticle provides at least a 2-fold increase, optionally a greater than 4-fold increase, in a tumor area under the curve (AUC) of at least one anti-cancer agent compared to administration of an equivalent amount of the at least one anti-cancer agent wherein the at least one anti-cancer agent is not associated with a nanoparticle and/or prodrug.

39. A method of treating cancer in a subject in need thereof, wherein the method comprises administering to the subject a pharmaceutical formulation of claim 21.
